(19) United States
(12) Patent Application Publication
(10) Pub. No.: US 2008/0254482 A1
(43) Pub. Date: Oct. 16, 2008

(54) AUTOIMMUNE DISEASE BIOMARKERS

(75) Inventors: Dawn R. Mattoon, Wallingford, CT (US); Barry Schweitzer, Cheshire, CT (US); David Alcorta, Chapel Hill, NC (US); Dhavalkumar Patel, Reinach (CH); Ronald Falk, Chapel Hill, NC (US)

Correspondence Address: GREENLEE WINNER AND SULLIVAN P C, 4875 PEARL EAST CIRCLE, SUITE 200, BOULDER, CO, 80301, US

(73) Assignee: Invitrogen Corporation, Carlsbad, CA (US)

(21) Appl. No.: 11/944,254

(22) Filed: Nov. 21, 2007

Related U.S. Application Data
(60) Provisional application No. 60/867,022, filed on Nov. 22, 2006.

(51) Int. Cl. G01N 33/564

(52) U.S. Cl. 435/7.1

(57) ABSTRACT

Provided herein are novel panels of biomarkers for the diagnosis of autoimmune diseases, and methods and kits for detecting these biomarkers in samples of individuals suspected of having an autoimmune disease. Also provided are methods of monitoring the progression of an autoimmune disease and methods of monitoring the efficacy and side effects of a treatment for an autoimmune disease.

### CROSS-REFERENCE TO RELATED APPLICATIONS

This application claims benefit of U.S. Provisional Application No. 60/867,022, filed Nov. 22, 2006, which is incorporated by reference in its entirety herein to the extent that there is no inconsistency with the present disclosure.

### BACKGROUND OF THE INVENTION

This invention generally relates to biomarkers associated with autoimmune diseases, specifically Rheumatoid Arthritis (RA), Systemic Lupus Erythematosus (SLE) and Anti-Neutrophil Cytoplasmic Antibody (ANCA) associated diseases, and methods, compositions and kits for the diagnosis, prognosis, and monitoring the progression of autoimmune diseases.

The development of autoantibodies is observed in autoimmune disorders and numerous cancers. Because of this, proteins targeted by autoantibodies (herein referred to as “autoantigens”) are effective biomarkers and form the basis of potential diagnostic and prognostic assays, as well as approaches for monitoring disease progression and response to treatment. The effective use of autoantigen biomarkers for these applications, however, is often contingent upon the identification of not one but multiple biomarkers. This is a consequence of the observation that the development of autoantibodies to any given protein is typically seen only in a fraction of patients (A. Fossa et al., Prostate 59, 440-7 (Jun. 1, 2004); S. S. Van Rhee et al., Blood 105, 3939-3944 (2005)). Current methods for the identification of autoantigens are cumbersome, technically challenging, have low sensitivity, and poor reproducibility. It is therefore cumbersome and time-consuming to identify panels of disease-specific markers that could facilitate diagnosing and treating diseases.

One widely utilized approach for autoantigen identification is SEREX: serological analysis of cDNA expression libraries. This approach is most appropriate for cancer autoantigen identification, and involves the generation of tumor-specific lambda GT11 cDNA expression libraries, followed by immunological screening of plaque lifts using patient sera. The SEREX approach was successfully used to identify the cancer autoantigen NY-ESO-1, a protein that is autoantigenic in ˜20-50% of patients overexpressing NY-ESO-1 (Y. T. Chen et al., Proc Natl Acad Sci USA 94, 1914-8 (1997)). However, while clearly useful, SEREX is not a high throughput approach, it is expensive, labor-intensive, requiring expertise in sophisticated molecular biological techniques, typically has a high false positive rate and, because it relies on bacterial protein expression, cannot identify autoantigens requiring post-translational modifications (U. Sahin et al., Proc Natl Acad Sci USA 92, 11810-3 (1995)). More recently, reverse phase protein microarrays have been used to identify colon cancer and lung cancer autoantigens (M. J. Nam et al., Proteomics 3, 2108-15 (2003); F. M. Brichory et al., Proc Natl Acad Sci USA 98, 9824-9 (2001)). These arrays are made by fractionating cancer cell homogenates, arraying them in spots on a microarray, probing them with patient sera, and detecting antibody binding. Mass-spectrometry based techniques are subsequently used to identify the actual autoantigen—a process which can be both time-consuming and tedious.

Functional protein microarrays are another method that may be used to identify biomarkers. These protein microarrays empower investigators with defined high-protein content for profiling serum samples to identify autoantigen biomarkers. Human protein microarrays may contain as many as 1800, 3000, 4000, 5000, 6000, 7000, 8000, 9000, 10,000, 100,000, 500,000 or 1,000,000 or more purified human proteins immobilized on nitrocellulose-coated glass slides. The protein microarrays may be probed with serum from a diseased individual to identify reactive proteins that are potential biomarkers for the disease. Human protein microarrays that contain proteins that are expressed in insect cells are expected to contain appropriate post-translational modifications. Because all proteins are purified under native conditions, immobilized proteins are expected to maintain their native conformations (B. Schweitzer, P. Predki, M. Snyder, Proteomics 3, 2190-9 (2003)).

Autoimmune diseases arise from an overactive immune response against the body's own cells and tissues. Today there are many human diseases classified as either definite or probable autoimmune diseases, the prominent examples being Systemic Lupus Erythematosus, Sjögren's syndrome and Rheumatoid Arthritis. The causes of autoimmune diseases are often unknown and the symptoms can appear without warning or apparent cause. Diagnosis of autoimmune diseases can be difficult because symptoms can vary greatly from person to person and are easily confused with other disorders. Diagnosis of autoimmune disorders largely rests on accurate medical history and physical examination of the patient in conjunction with abnormalities observed in routine laboratory tests. In several systemic disorders, serological assays which can detect specific autoantibodies can be employed. However, current tests are often inconclusive and inaccurate. The ability to screen a patient for multiple biomarkers associated with autoimmune diseases would improve diagnosis and treatment of the diseases.

Rheumatoid arthritis (RA) is a chronic, inflammatory autoimmune disease that causes the immune system to attack the joints. It is a disabling and painful inflammatory condition, which can lead to substantial loss of mobility due to pain and joint destruction. The disease is also systemic in that it often also affects many extra-articular tissues throughout the body including the skin, blood vessels, heart, lungs, and muscles. Rheumatoid arthritis can be difficult to diagnose. Symptoms differ from person to person and can be more severe in some people than in others. Within the same person, the full range of symptoms may develop over time, and only a few symptoms may be present in the early stages. Also, symptoms can be similar to those of other types of arthritis and joint conditions, and it may take some time for other conditions to be ruled out. Additionally, there is no single test for the disease. One common test used to help diagnose RA is for rheumatoid factor, an antibody that is present eventually in the blood of most people with the disease. Not all people with RA test positive for rheumatoid factor, however, especially early in the disease. Also, some people test positive for rheumatoid factor, yet never develop the disease. Another test assesses the presence of anti-citrullinated protein (ACP) antibodies. Other common laboratory tests include a white blood cell count, a blood test for anemia, and a test of the erythrocyte sedimentation rate, which measures inflammation in the body.

Systemic lupus erythematosus (SLE or lupus) is a chronic, potentially debilitating or fatal autoimmune disease in which the immune system attacks the body's cells and tissue, resulting in inflammation and tissue damage. SLE can affect any part of the body, but often harms the heart, joints (rheumatological), skin, kidneys, lungs, blood vessels and brain/nervous system. Some of the most common symptoms of the disease include extreme fatigue, painful or swollen joints (arthritis), unexplained fever, skin rashes, and kidney problems; however, no two cases of lupus are exactly alike. Signs and symptoms vary considerably from person to person, may come on suddenly or develop slowly, may be mild or severe, and may be temporary or permanent. Even the distinctive rash that gives the disease its name does not occur in every case. Additionally, the problems associated with the disease change over time and overlap with those of many other disorders. For these reasons, doctors may not initially consider lupus until the signs and symptoms become more obvious. Even then, lupus can be challenging to diagnose because nearly all people with lupus experience fluctuations in disease activity. Lupus can be effectively treated with drugs, mainly with immunosuppression, though there is currently no cure for this disease.

Currently, no single test can determine whether a person has lupus, but several laboratory tests may help a physician to make a diagnosis. For example, the antinuclear antibody (ANA) test is commonly used to look for autoantibodies that react against components of the cell nucleus. Most people with lupus test positive for ANA; however, there are a number of other causes of a positive ANA besides lupus, including infections, other autoimmune diseases, and a positive ANA may occasionally be found in healthy individuals. The ANA test is thus not definitive for lupus, but is only one of a number of considerations used in making a diagnosis. Other laboratory tests are used to monitor the progress of lupus or its symptoms, once it has been diagnosed. A complete blood count, urinalysis, blood chemistries, and the erythrocyte sedimentation rate (ESR) test can provide valuable information on the stage or progression of the disease. Another common test measures the blood level of proteins of the complement system. People with lupus often have increased ESRs and low complement levels, especially during flare-ups of the disease.

Anti-neutrophil cytoplasmic antibodies (ANCAs) are antibodies against molecules in the cytoplasm of neutrophil granulocytes and monocyte lysosomes (Niles et al., Arch Intern Med 156, 440-5 (1996)). They are detected in a number of autoimmune disorders, but are particularly associated with systemic vasculitis. ANCA-associated vasculitis is the most common primary systemic small-vessel vasculitis to occur in adults (I. Mansi, A. Opran, and F. Rosner, American Family Physician 65, 1615-20 (2002)). ANCA-associated small-vessel vasculitis includes microscopic polyangiitis, Wegener's granulomatosis, Churg-Strauss syndrome, and drug-induced vasculitis. Rapid diagnosis of ANCA-associated diseases is critically important, because life-threatening injury to organs often develops quickly and is mitigated dramatically by immunosuppressive treatment. Less than 10% of patients with clinically and pathologically identical diseases do not have ANCA, and at least 90% of patients with Wegener's granulomatosis, microscopic polyangiitis, and the Churg-Strauss syndrome have either MPO-ANCA or PR3-ANCA (R. Falk and J. C. Jennette, J Am Soc Nephrol 13, 1977-1979 (2002)). Thus, there is a loose correlation between ANCA titer and disease activity; however, these studies may be hampered by the imprecision of the ANCA assays themselves. In general, serologic testing for ANCA is recommended for patients with glomerulonephritis, pulmonary hemorrhage, especially pulmonary-renal syndrome, cutaneous vasculitis with systemic features, mononeuritis multiplex or other peripheral neuropathy, long-standing sinusitis or otitis, subglottic tracheal stenosis, and retro-orbital mass.

The ability to screen a patient for multiple biomarkers associated with autoimmune diseases would improve diagnosis and treatment of the diseases. However, it is unlikely that a single individual marker can accomplish this task. Assay experience with autoimmune diseases and cancer patients has demonstrated that a single antigen is not sufficient to characterize all sera and to differentiate between healthy and diseased individuals. An approach that can identify as many autoimmune biomarkers as possible to generate a serological test will be beneficial so that patients can be selected for therapy based on accurate information regarding their antigenic profile. There is a need in the art for the identification of new biomarkers that can be used in the care and management of autoimmune diseases, for example by the development of a non-invasive, accurate, fast and sensitive assay that utilizes multiple biomarkers for the detection, diagnosis, staging, and monitoring of autoimmune diseases in individuals.

### SUMMARY OF THE INVENTION

The present invention recognizes the need for a reliable test for autoimmune diseases, and in particular for a minimally invasive test that can detect RA, SLE and ANCA.

The invention is based in part on the discovery of a collection of autoantibody biomarkers for the detection, diagnosis, prognosis, staging, and monitoring of RA, SLE and ANCA. The invention provides biomarkers for autoimmune disease, particularly autoantibody biomarkers, and biomarker detection panels. Furthermore, the invention provides methods of detecting, diagnosing, prognosing, staging, and monitoring RA, SLE and ANCA by detecting biomarkers of the invention in a test sample of an individual.

The present invention identifies numerous biomarkers that are useful for the detection, diagnosis, staging, and monitoring of autoimmune diseases in individuals. A determination of the presence or absence of an autoimmune disease in an individual does not necessarily require that antibodies against all of the identified antigen biomarkers are present or absent. Similarly, a determination of the presence or absence of an autoimmune disease in an individual does not require that all of the target antigens biomarkers be present in increased or decreased amounts. Art-recognized statistical methods can be used to determine the significance of a specific pattern of antibodies against a plurality of the listed antigen biomarkers, or the significance of a specific pattern of increased or decreased amounts of biomarkers.

In one aspect of the invention, serum from patients diagnosed with RA, SLE and ANCA as well as healthy patients were profiled against a human protein microarray containing thousands of human proteins used as biomarkers. Numerous proteins on the array were bound by antibodies from patients diagnosed with RA, SLE and ANCA, but not healthy patients. Many of the proteins were selective for RA, SLE or ANCA antibodies showing little or no binding in one or both of the other disease groups. Additionally, serum from patients diagnosed with RA were profiled against a high throughput human protein microarray before and after treatment with a drug used to treat auto-immune disorders. Several proteins had altered patient antibody levels after treatment compared to the antibody levels for the target proteins before treatment.

One embodiment of the invention is a method of detecting autoantibodies in a test sample from an individual suspected of having an autoimmune disease by contacting the test sample from the individual with one or more target antigens each comprising an autoantigen of Table 1 (provided below) or a fragment thereof comprising an epitope; and detecting binding of the one or more target antigens, wherein the binding of the one or more target antigens detects the presence of the one or more antibodies in the test sample. In a further embodiment, at least 10%; at least 25%; at least 50%; at least 80%; or at least 95% of the target antigens are bound by one or more antibodies from the test sample. The sample used in the detection and diagnosis methods of the invention can be any type of sample, but preferably is a saliva sample or a blood sample, or a fraction thereof, such as plasma or serum.

Another embodiment is a method of diagnosing RA in an individual comprising contacting a test sample from the individual with one or more target antigens and detecting binding of the one or more target antigens to one or more antibodies in the test sample, wherein the presence of the one or more antibodies against the one or more target antigens is indicative of rheumatoid arthritis, wherein the one or more target antigens are selected from the group comprising of Table 2 (as provided below) or a fragment thereof comprising an epitope.

Another embodiment is a method of diagnosing SLE in an individual comprising contacting a test sample from the individual with one or more biomarkers; and detecting binding of the one or more biomarkers to one or more antibodies in the test sample, wherein the presence of the one or more antibodies against the one or more biomarkers is indicative of SLE, wherein the one or more biomarkers are selected from the group comprising of Table 3 (as provided below) or a fragment thereof comprising an epitope.

Another embodiment is a method of diagnosing ANCA in an individual comprising contacting a test sample from the individual with one or more target antigens; and detecting binding of the one or more target antigens to one or more antibodies in the test sample, wherein the presence of the one or more antibodies against the one or more target antigens is indicative of ANCA, wherein the one or more target antigens are selected from the group comprising of Table 5 (as provided below) or a fragment thereof comprising an epitope.

Another embodiment of the present invention is a composition comprising one or more human antibodies from an individual with an autoimmune disease, wherein each antibody is bound to one or more target antigens each comprising an autoantigen of Table 1 or fragments thereof comprising an epitope. The target antigens may be immobilized on a solid support or may be part of a protein microarray. Another embodiment of the present invention is a solid support comprising two or more target antigens each comprising an autoantigen of Table 1 or fragments thereof comprising an epitope; and an immobilized human antibody control, wherein the human antibody control is a positive control for immunodetection.

The invention also provides kits that include one or more test antigens or one or more target antigens provided herein. The kits can include one or more reagents for detecting binding of an antibody from a sample. In some embodiments, the one or more test antigens or one or more target antigens of a kit are provided bound to a solid support. The invention includes kits that include biomarker detection panels of the invention, including biomarker detection panels in which the target antigens are bound to one or more solid supports. In some embodiments of kits, the kit provides a biomarker detection panel in which the target antigens of the detection panel are bound to a chip or array.

In some embodiments, the invention provides compositions, kits and methods for detecting one or more identified biomarkers as a diagnostic indicator for an autoimmune disease, such as RA, SLE, or ANCA. Additional uses of the invention include, among others: 1) the detection of one or more identified antigen biomarkers as a tool to select an appropriate therapeutic approach for treatment of a patient with a disease; 2) the use of one or more detected biomarkers as a vaccine candidate or therapeutic target; 3) the use of one or more identified biomarkers as a screening tool for use in the development of new therapeutics including antibodies; 4) the detection of one or more identified biomarkers as a tool for stratifying patients prior to infliximab (Remicade®) treatment; 5) the detection of one or more identified biomarkers for the early identification of the development of an SLE-like response in RA patients undergoing infliximab treatment; 6) the detection of observed anti-TNFα autoantibody response for the development of improved anti-TNF therapies; and 7) the detection of observed anti-TNFα autoantibody response as a surrogate marker for monitoring patient immune response to infliximab therapy.

### BRIEF DESCRIPTION OF THE DRAWINGS

FIG. 1 shows a protein microarray comprised of more than 5,000 purified human proteins arrayed in duplicate on nitrocellulose-coated glass slides. Array features are arranged in 48 distinct subarrays, each of which includes unique human proteins and common control elements. An individual subarray is shown in the right panel.

FIG. 2A shows a panel of 12 samples, including sera from healthy donors, as well as lupus, ANCA, and rheumatoid arthritis patients profiled on the 5,000 protein microarrays of FIG. 1 at three dilutions and the distribution of signals evaluated. Signal intensity is plotted as a function of the number of features giving rise to signals in the specified range. FIG. 2B shows the average background signals plotted for each dilution.

FIGS. 3A-3C show three-part statistical analysis of protein microarray data. Background-subtracted signal intensity data was evaluated using three independent statistical approaches, including M-statistics applied to quantile normalized data (FIG. 3A), volcano analysis applied to non-normalized data (FIG. 3B), and fold change calculations applied to quantile normalized data (FIG. 3C). Candidate biomarkers were selected based on the indicated threshold values developed for each analytical measure. The overlap in candidate autoantigens identified using each statistical approach is shown.

FIG. 4 shows signals from immunoreactive proteins identified in the SLE or healthy population based on either M-statistics or volcano analysis (classification statistic). Proteins ranking in the top 100 on the custom array assays were evaluated against the original 5,000-protein array data to assess the reproducibility of immunoreactive signals. The number of proteins with a calculated p-value<0.01 or a Signal Used difference>1500 that were included on the focused arrays are indicated (solid bars). Values calculated from the custom array data were used to generate a rank order, and proteins ranking in the top 100 on the custom arrays, sorted by either p-value or Signal Used difference, are indicated with hatched bars. The percentage of proteins identified as significant in the original assays that are also in the top 100 on the custom arrays (by each metric) are indicated.

FIG. 5 illustrates separation of populations using Principle Component Analysis. Principle component analysis was carried out on non-normalized signal intensity data derived from all 5,000 human proteins (left panel), a set of 10 SLE-annotated autoantigens (middle panel), or a set of 18 candidate autoantigens (right panel). Three-dimensional representations of the first three principle components are shown. To ensure accurate reporting of the data, each plot is represented as two 180 degree planar rotations. Black spots correspond to normal samples, red spots correspond to SLE samples. Depth cues are provided through changes in color intensity (black to gray and red to pink).

FIGS. 6A-6C show immunological profiling using Luminex® technology. FIG. 6A shows Luminex® beads from four color regions coupled to goat anti-GST antibody. Anti-GST-conjugated beads from one region were incubated in independent reactions with increasing concentrations of purified recombinant GST. Beads from all four regions were then mixed together and incubated with a second fluorescently labeled anti-GST antibody. Signals were obtained from each bead region and plotted as a function of GST concentration. FIG. 6B shows Luminex® beads from eighteen color regions were coupled to goat anti-GST antibody. Anti-GST-conjugated beads from all color regions were incubated in independent reactions with purified recombinant GST-tagged candidate autoantigens. Beads from all regions were then mixed together and incubated with increasing dilutions of serum samples in duplicate. Serum IgG bound to the GST-tagged proteins was detected using a fluorescently labeled anti-human IgG antibody. Median Fluorescence Intensity data for one representative protein is plotted across all serum dilutions. Error bars indicate standard deviations calculated across the duplicate assays. FIG. 6C shows Pearson's Correlation Coefficients calculated from the Median Fluorescence Intensity data generated through Experiment 5 relative to the background-subtracted signal intensity data generated through immunological profiling on the custom arrays.

### DETAILED DESCRIPTION OF THE INVENTION

The invention is based on the identification of autoantigens for autoimmune diseases. Serum samples from healthy individuals as well as patients with autoimmune diseases, such as RA, SLE, and ANCA were profiled on ProtoArray™ human protein microarrays (Invitrogen Corporation, Carlsbad, Calif.), to identify multiple disease-specific biomarkers. The extensive content of the arrays, including lower abundance proteins, native conformation, and insect cell-derived post-translational modifications, enabled the identification of biomarkers not previously known to be associated with RA, SLE and/or ANCA.

A list of antigen biomarkers (profiled using the ProtoArray™ human protein microarray) that were bound by antibodies from sera from patients diagnosed with an autoimmune disease is shown in Table 1. Proteins that were bound by antibodies from RA, SLE, and ANCA patients, which were not present in normal, healthy individuals, are shown in Tables 2, 3 and 5, respectively. Microarrays, or other assay formats, containing these biomarkers are able to detect the presence of antibodies in a patient sample that bind the biomarkers, enabling the diagnosis and monitoring of the diseases. Microarrays or other assays can contain specific biomarkers or a specific group of biomarkers, such as those associated with RA in Table 2, for detection of antibodies for a specific disease.

One embodiment of the present invention is a method of detecting one or more target antibodies in a test sample of an individual suspected of having an autoimmune disease comprising: a) contacting the test sample from the individual with one or more target antigens each comprising an autoantigen of Table 1 or a fragment thereof comprising an epitope; and b) detecting binding of the one or more target antigens, wherein the binding of the one or more target antigens detects the presence of the one or more target antibodies in the test sample. In a further embodiment, the test sample is contacted with two or more; ten or more; twenty or more; fifty or more; or all of the autoantigens of Table 1 or fragments thereof comprising an epitope. In a further embodiment, the quantitative amount of antibodies that bind to each biomarker is determined.

In a further embodiment, at least 1, 2, 3, 4, 5, 10, 20, 35, 50, 75, 100, 150 or 200 antigen biomarkers must be bound by an antibody from the test sample to indicate the presence of an autoimmune disease.

Autoimmune diseases, including RA, SLE and ANCA, will have several autoantigens in common with other autoimmune diseases. Autoimmune diseases will also have antigens that are selective for that particular autoimmune disease. The binding of one or more of the autoantigens from Table 1 by an antibody from a patient's test sample will indicate the presence of an autoimmune disease. However, binding of one or more specific autoantigens selective for a particular autoimmune disease may be required to determine which autoimmune disease is present.

Another embodiment of the present invention is a method of diagnosing rheumatoid arthritis in an individual comprising: a) contacting a test sample from the individual with one or more target antigens, each comprising an autoantigen of Table 2 or a fragment thereof comprising an epitope; and b) detecting binding of the one or more target antigens to one or more antibodies in the test sample, wherein the presence of the one or more antibodies bound against the one or more target antigens is indicative of rheumatoid arthritis. In a further embodiment, the test sample is contacted with two or more; ten or more; twenty or more; or all of the autoantigens listed in Table 2 or fragment thereof comprising an epitope. In a further embodiment, the amount of antibodies that bind to each antigen is determined.

In a further embodiment, at least 1, 2, 3, 4, 5, 10, 20, or 35 of the RA antigens are bound by an antibody from the test sample to indicate the presence of rheumatoid arthritis. One autoantigen, leukocyte receptor cluster member 12 (BC033195) is selective for RA but not SLE or ANCA. In a further embodiment, a kit and a method for diagnosing RA comprises contacting a test sample with one or more autoantigens, wherein one of the biomarkers is leukocyte receptor cluster member 12.

Another embodiment of the present invention is a method of diagnosing systemic lupus erythematosus in an individual comprising: a) contacting a test sample from the individual with one or more target antigens, each comprising an autoantigen of Table 3 or fragments thereof comprising an epitope; and b) detecting binding of the one or more target antigens to one or more antibodies in the test sample, wherein the presence of the one or more antibodies bound against the one or more target antigens is indicative of systemic lupus erythematosus. In a further embodiment, the test sample is contacted with two or more; ten or more; twenty or more; fifty or more; or all of the autoantigens listed in Table 3. In a further embodiment, the amount of antibodies that bind to each antigen is determined.

In a further embodiment, at least 1, 2, 3, 4, 5, 10, 20, 35, 50, 75, 100 or 150 of the SLE antigens are bound by an antibody from the test sample to indicate the presence of systemic lupus erythematosus. In a further embodiment, a kit and a method for diagnosing SLE comprises contacting a test sample with one or more antigens, wherein one or more of the antigens are selected from the autoantigens in Table 4 or fragments thereof comprising an epitope.

Another embodiment of the present invention is a method of diagnosing anti-neutrophil cytoplasmic antibody associated diseases in an individual comprising: a) contacting a test sample from the individual with one or more target antigens, each comprising an autoantigen of Table 5 or fragments thereof comprising an epitope; and b) detecting binding of the one or more target antigens to one or more antibodies in the test sample, wherein the presence of the one or more antibodies bound against the one or more target antigens is indicative of anti-neutrophil cytoplasmic antibody associated diseases. In a further embodiment, the test sample is contacted with two or more; ten or more; twenty or more; fifty or more; or all of the autoantigens listed in Table 5. In a further embodiment, the amount of antibodies that bind to each antigen is determined.

In a further embodiment, at least 1, 2, 3, 4, 5, 10, 20, 35, 50, 75, or all of the ANCA autoantigens are bound by an antibody from the test sample to indicate the presence of anti-neutrophil cytoplasmic antibody associated diseases. In a further embodiment, a kit and a method for diagnosing ANCA comprises contacting a test sample with one or more antigens, wherein one or more of the antigens are selected from the autoantigens in Table 6 or fragments thereof comprising an epitope.

The progression or remission of a disease can be monitored by contacting test samples from an individual taken at different times with the panel of antigens. For example, a second test sample is taken from the patient and contacted with the antigen panel days or weeks after the first test sample. Alternatively, the second or subsequent test samples can be taken from the patient and tested against the panel of antigens at regular intervals, such as daily, weekly, monthly, quarterly, semi-annually, or annually. By testing the patient's test samples at different times, the presence of antibodies and therefore the stage of the disease can be compared. A further embodiment of the invention is a method of monitoring one or more target antibodies in test samples from an individual diagnosed as having an autoimmune disease comprising: a) contacting a first test sample from the individual with a first set of one or more target antigens; b) detecting binding of the one or more target antigens, wherein the binding of the one or more target antigens detects the presence of the one or more target antibodies in the first test sample; c) contacting a second test sample from the individual with a second set of the one or more target antigens; d) detecting binding of the one or more target antigens, wherein the binding of the one or more target antigens detects the presence of the one or more target antibodies in the second test sample; and e) comparing the presence of the one or more antibodies bound against the one or more target antigens from the first test sample with the one or more antibodies bound against the one or more target antigens from the second test sample, wherein each of the one or more target antigens comprises an autoantigen of Table 1 or fragments thereof comprising an epitope. In other embodiments of the invention, the one or more target antigens comprise an autoantigen of Table 2, Table 3, or Table 5 or fragments thereof.

The progression of the disease is further monitored by quantitatively comparing the amounts of antibodies that bind to the autoantigens. Accordingly, another embodiment of the invention further comprises detecting the amount of the one or more antibodies against the one or more antigens in the first test sample and the second test sample; and comparing the amount of the one or more antibodies from the first test sample with the amount of the one or more antibodies from the second test sample.

Another embodiment of the invention is a mixture comprising one or more target antigens each comprising an autoantigen of Table 1 or a fragment thereof comprising an epitope; and a test sample from an individual suspected of having an autoimmune disease. The mixture optionally further comprises a control antibody against one or more of the target antigens. In a further embodiment, the mixture comprises two or more; ten or more; twenty or more; fifty or more; one hundred or more; or all of the autoantigens of Table 1 or fragments thereof comprising an epitope. The test sample includes, but is not limited to, cells, tissues, or bodily fluids from an individual.

The present invention identifies >300 proteins that are selectively recognized by antibodies in RA, ANCA, or SLE patient sera which represent an important pool of novel candidates for potential diagnostic markers or therapeutic targets. The present invention further identifies a panel of antigens that exhibit increased or decreased autoantibody response in RA patients following infliximab (Remicade®) treatment, which represents an important group of novel biomarkers for utility in patient stratification and monitoring treatment efficacy. These proteins also can facilitate early identification of patients progressing towards infliximab-induced SLE-like syndrome.

Infliximab (Remicade®) is an injectable antibody used to treat autoimmune disorders like Crohn's disease, ulcerative colitis, psoriatic arthritis and rheumatoid arthritis. The drug reduces the amount of active TNF-α (tumour necrosis factor alpha) in the body by binding to it and preventing it from signaling the receptors for TNF-A on the surface of cells. Autoantibodies directed against the cytokine tumor necrosis factor alpha (TNF-α) comprise the most statistically significant differentiator of untreated RA patients relative to patients after 20 weeks of infliximab treatment. Detection of an anti-TNFα autoantibody response serves as a tool for improvements to anti-TNF antibody-based therapies, the development of adjuvant therapies designed to mitigate this response, as well as a marker for monitoring host-response to infliximab.

Infliximab has also been reported to be helpful in reducing the joint inflammation of juvenile rheumatoid arthritis, ankylosing spondylitis, uveitis, psoriasis, and for sarcoidosis that is not responding to traditional therapies. Treatment with infliximab may increase the risk of developing certain types of cancer or autoimmune disorders (such as a lupus-like syndrome).

Another embodiment of the invention comprises a method of monitoring one or more target antibodies in test samples from an individual receiving treatment for an autoimmune disease comprising a) contacting a first test sample from an individual with a first set of one or more target antigens; b) detecting binding of the one or more target antigens to one or more antibodies in the first test sample, wherein the presence of the one or more antibodies bound against the one or more target antigens detects the one or more target antibodies; c) administering a treatment for the autoimmune disease to the individual; d) after the administration of the treatment, contacting a second test sample from the individual with a second set of the one or more target antigens; e) detecting binding of the one or more target antigens to one or more antibodies in the second test sample, wherein the presence of the one or more antibodies bound against the one or more target antigens detects the one or more target antibodies; and f) comparing the presence of the one or more antibodies against the one or more target antigens from the first sample with the one or more antibodies against the one or more target antigens from the second sample, wherein each of the one or more target antigens comprises an autoantigen of Table 1 or fragments thereof comprising an epitope.

The binding levels of the antibodies to the one or more antigens may increase or decrease as a result of the treatment. In one embodiment, the decrease of binding levels to autoantigens of Table 7A is indicative of the presence of autoimmune disease in the patient. In another embodiment, the increase of binding levels to autoantigens of Table 7B is indicative of the presence of autoimmune disease.

By administering treatment, it is meant to encompass any therapeutic drug, procedure, or combination thereof administered to a patient to alleviate an autoimmune disease, including, but not limited to, administering a drug orally or intravenously to a patient. Where the autoimmune disease is rheumatoid arthritis, the treatment may comprise intravenously administering the drug infliximab to the patient. The treatment may be continuous, that is, administered to the patient at regular intervals. Multiple test samples can be taken from the patient during the course of the treatment. Preferably, the first test sample is taken from the patient before treatment begins.

In a further embodiment, the amount of the one or more antibodies against the one or more antigens in each test sample is detected; and the amount of the one or more antibodies from the first test sample is compared with the amount of one or more antibodies from the second test sample.

In one embodiment, the treatment is for rheumatoid arthritis and the one or more target antigens each comprise an autoantigen of Table 2 or a fragment thereof comprising an epitope. Preferably, the treatment is the administration of infliximab to a patient.

The invention also provides a method of staging autoimmune disease in an individual. This method comprises identifying a human patient having an autoimmune disease and analyzing cells, tissues or bodily fluid from such human patient for the autoimmune disease-associated biomarkers of the present invention. The presence or level of the biomarker is then compared to the level of the biomarker in the same cells, tissues or bodily fluid type of a healthy control individual, or with a reference range of the level of biomarker obtained from at least one healthy control individual. An elevated level of immune reactivity against a biomarker protein identified as being present in elevated amounts in autoimmune disease patients, when compared to the control or reference range, is associated with the presence of autoimmune disease in the test individual. A decreased level of immune reactivity against a biomarker protein identified as being present in decreased amounts in autoimmune disease patients, when compared to the control or reference range, is associated with the presence of autoimmune disease in the test individual.

### DEFINITIONS

The term “about” as used herein refers to a value within 10% of the underlying parameter (i.e., plus or minus 10%), and is sometimes a value within 5% of the underlying parameter (i.e., plus or minus 5%), a value sometimes within 2.5% of the underlying parameter (i.e., plus or minus 2.5%), or a value sometimes within 1% of the underlying parameter (i.e., plus or minus 1%), and sometimes refers to the parameter with no variation. Thus, a distance of “about 20 nucleotides in length” includes a distance of 19 or 21 nucleotides in length (i.e., within a 5% variation) or a distance of 20 nucleotides in length (i.e., no variation) in some embodiments.

As used herein, the article “a” or “an” can refer to one or more of the elements it precedes (e.g., a protein microarray “a” protein may comprise one protein sequence or multiple proteins).

The term “or” is not meant to be exclusive to one or the terms it designates. For example, as it is used in a phrase of the structure “A or B” may denote A alone, B alone, or both A and B.

By “biomarker” it is meant a biochemical characteristic that can be used to detect, diagnose, prognose, direct treatment, or to measure the progress of a disease or condition, or the effects of treatment of a disease or condition. Biomarkers include, but are not limited to, the presence of a nucleic acid, protein, carbohydrate, or antibody, or combination thereof, associated with the presence of a disease in an individual. The present invention provides biomarkers for RA, SLE and ANCA that are antibodies present in the sera of subjects diagnosed with RA, SLE and ANCA. The biomarker antibodies in the present invention are the autoantibodies displaying increased reactivity in individuals with an autoimmune disease, most likely as a consequence of their increased abundance. The autoantibodies can be detected with autoantigens, human proteins that are specifically bound by the antibodies. Importantly, biomarkers need not be expressed in a majority of disease individuals to have clinical value. The receptor tyrosine kinase Her2 is known to be over-expressed in approximately 25% of all breast cancers (J. S. Ross et al., Mol Cell Proteomics 3, 379-98 (April, 2004)), and yet is a clinically important indicator of disease progression as well as specific therapeutic options.

“Biomolecule” refers to an organic molecule of biological origin, e.g., steroids, fatty acids, amino acids, nucleotides, sugars, peptides, polypeptides, antibodies, polynucleotides, complex carbohydrates or lipids.

The phrase “differentially present” refers to differences in the quantity of a biomolecule (such as an antibody) present in a sample taken from patients having an autoimmune disease as compared to a comparable sample taken from patients who do not have an autoimmune disease (e.g., normal or healthy patients). A biomolecule is differentially present between the two samples if the amount of the polypeptide in one sample is significantly different from the amount of the polypeptide in the other sample. For example, a polypeptide is differentially present between the two samples if it is present in an amount (e.g., concentration, mass, molar amount, etc.) at least about 150%, at least about 200%, at least about 500% or at least about 1000% greater or lesser than it is present in the other sample, or if it is detectable (gives a signal significantly greater than background or a negative control) in one sample and not detectable in the other. Any biomolecules that are differentially present in samples taken from autoimmune disease patients as compared to subjects who do not have an autoimmune disease can be used as biomarkers.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′.sub.2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region. An “autoantibody” is an antibody that is directed against the host's own proteins or other molecules. In the present invention, high throughput microarrays have been used to detect autoantibodies from RA, SLE and ANCA patients that are not typically present in normal patients.

The term “antigen” or “test antigen” as used herein refers to proteins or polypeptides to be used as targets for screening test samples obtained from subjects for the presence of autoantibodies. “Autoantigen” is used to denote antigens for which the presence of antibodies in a sample of an individual has been detected. These antigens, test antigens, or autoantigens are contemplated to include any fragments thereof of the so-identified proteins, in particular, immunologically detectable fragments. They are also meant to include immunologically detectable products of proteolysis of the proteins, as well as processed forms, post-translationally modified forms, such as, for example, “prep” “pro,” or “prepro” forms of markers, or the “prep” “pro,” or “prepro” fragment removed to form the mature marker, as well as allelic variants and splice variants of the antigens, test antigens, or autoantigens. The identification or listing of antigens, test antigens, and autoantigens also includes amino acid sequence variants of these, for example, sequence variants that include a fragment, domain, or epitope that shares immune reactivity with the identified antigen, test antigen, and autoantigen protein. Similarly, an “autoantigen” refers to a molecule, such as a protein, endogenous to the host that is recognized by an autoantibody.

An “epitope” is a site on an antigen, such as an autoantigen disclosed herein, recognized by an antibody.

As used herein, the word “protein” refers to a full-length protein, a portion of a protein, or a peptide. Proteins can be produced via fragmentation of larger proteins, or chemically synthesized. Proteins may, for example, be prepared by recombinant overexpression in a species such as, but not limited to, bacteria, yeast, insect cells, and mammalian cells. Proteins to be placed in a protein microarray of the invention, may be, for example, are fusion proteins, for example with at least one affinity tag to aid in purification and/or immobilization. In certain aspects of the invention, at least 2 tags are present on the protein, one of which can be used to aid in purification and the other can be used to aid in immobilization. In certain illustrative aspects, the tag is a His tag, a GST tag, or a biotin tag. Where the tag is a biotin tag, the tag can be associated with a protein in vitro or in vivo using commercially available reagents (Invitrogen, Carlsbad, Calif.). In aspects where the tag is associated with the protein in vitro, a Bioease tag can be used (Invitrogen, Carlsbad, Calif.).

As used herein, the term “peptide,” “oligopeptide,” and “polypeptide” are used interchangeably with protein herein and refer to a sequence of contiguous amino acids linked by peptide bonds. As used herein, the term “protein” refers to a polypeptide that can also include post-translational modifications that include the modification of amino acids of the protein and may include the addition of chemical groups or biomolecules that are not amino acid-based. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

A “variant” of a polypeptide or protein, as used herein, refers to an amino acid sequence that is altered with respect to the referenced polypeptide or protein by one or more amino acids. In the present invention, a variant of a polypeptide retains the antigenicity, or antibody-binding property, of the referenced protein. In preferred aspects of the invention, a variant of a polypeptide or protein can be bound by the same population of autoantibodies that are able to bind the referenced protein. Preferably a variant of a polypeptide has at least 60% identity to the referenced protein over a sequence of at least 15 amino acids. More preferably a variant of a polypeptide is at least 70% identical to the referenced protein over a sequence of at least 15 amino acids. Protein variants can be, for example, at least 80%, at least 90%, at least 95%, or at least 99% identical to referenced polypeptide over a sequence of at least 15 amino acids. Protein variants of the invention can be, for example, at least 80%, at least 90%, at least 95%, or at least 99% identical to referenced polypeptide over a sequence of at least 20 amino acids. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may also include amino acid deletions or insertions, or both. Guidance in determining which amino acid residues may be substituted, inserted, or deleted without abolishing immunological reactivity may be found using computer programs well known in the art, for example, DNASTAR software.

Protein biomarkers used in a protein array of the present invention may be the full protein or fragments of the full protein. Protein fragments are suitable for use as part of the protein array as long as the fragments contain the epitope recognized by the antibodies. The required epitope for a given full protein can be mapped using protein microarrays, and with ELISPOT or ELISA techniques. It is understood that the antigen biomarkers provided by the present invention are meant to encompass the full protein as well as fragments thereof comprising an epitope. Typically, suitable protein fragments comprise at least 5%; at least 10%; at least 20%; or at least 50% of the full length protein amino acid sequence. In one embodiment of the present invention, protein fragments of target autoantigens contain at least 6 contiguous amino acids; at least 10 contiguous amino acids; at least 20 contiguous amino acids; at least 50 contiguous amino acids; at least 100 contiguous amino acids; or at least 200 contiguous amino acids of the full length protein.

As used herein, a “biomarker detection panel” or “biomarker panel” refers to a set of biomarkers that are provided together for detection, diagnosis, prognosis, staging, or monitoring of a disease or condition, based on detection values for the set (panel) of biomarkers.

The methods of the present invention are carried out on test samples derived from patients, including individuals suspected of having an autoimmune disease and those who have been diagnosed to have a disease. A “test sample” as used herein can be any type of sample, such as a sample of cells or tissue, or a sample of bodily fluid, preferably from an animal, most preferably a human. The sample can be a tissue sample, such as a swab or smear, or a pathology or biopsy sample of tissue, including tumor tissue. Samples can also be tissue extracts, for example from tissue biopsy or autopsy material. A sample can be a sample of bodily fluids, such as but not limited to blood, plasma, serum, sputum, semen, synovial fluid, cerebrospinal fluid, urine, lung aspirates, nipple aspirates, tears, or a lavage. Samples can also include, for example, cells or tissue extracts such as homogenates, cell lysates or solubilized tissue obtained from a patient. A preferred sample is a blood or serum sample.

By “blood” is meant to include whole blood, plasma, serum, or any derivative of blood. A blood sample may be, for example, serum.

A “patient” is an individual diagnosed with a disease or being tested for the presence of disease. A patient tested for a disease can have one or more indicators of a disease state, or can be screened for the presence of disease in the absence of any indicators of a disease state. As used herein an individual “suspected” of having a disease can have one or more indicators of a disease state or can be part of a population routinely screened for disease in the absence of any indicators of a disease state.

Autoimmune diseases are diseases characterized by an immune response against the body's own cells and tissues. Rheumatoid arthritis (RA) is a chronic, inflammatory autoimmune disease that causes the immune system to attack the joints. Systemic lupus erythematosus (SLE or lupus) is a chronic, potentially debilitating or fatal autoimmune disease in which the immune system attacks the body's cells and tissue, resulting in inflammation and tissue damage. ANCA refers to any autoimmune disease characterized by the presence of anti-neutrophil cytoplasmic antibodies, such as small-vessel vasculitis and including, but not limited to, microscopic polyangiitis, Wegener's granulomatosis, Churg-Strauss syndrome, and drug-induced vasculitis.

By “an individual suspected of having an autoimmune disease,” is meant an individual who has been diagnosed with an autoimmune disease, such as RA, SLE or ANCA, or who has at least one indicator of autoimmune disease, or who is at an increased risk of developing autoimmune disease due to age, environmental and/or nutritional factors, or genetic factors.

As used herein, the term “array” refers to an arrangement of entities in a pattern on a substrate. Although the pattern is typically a two-dimensional pattern, the pattern may also be a three-dimensional pattern. In a protein array, the entities are proteins. In certain embodiments, the array can be a microarray or a nanoarray. A “nanoarray” is an array in which separate entities are separated by 0.1 nm to 10 μm, for example from 1 nm to 1 μm. A “microarray” is an array in the density of entities on the array is at least 100/cm². On microarrays separate entities can be separated, for example, by more than 1 μm.

The term “protein array” as used herein refers to a protein array, a protein microarray or a protein nanoarray. A protein array may include, for example, but is not limited to, a “ProtoArray™,” protein high density array (Invitrogen, Carlsbad, Calif., available on the Internet at Invitrogen.com). The ProtoArray™ high density protein array can be used to screen complex biological mixtures, such as serum, to assay for the presence of autoantibodies directed against human proteins. Alternatively, a custom protein array that includes autoantigens, such as those provided herein, for the detection of autoantibody biomarkers, can be used to assay for the presence of autoantibodies directed against human proteins. In certain disease states including autoimmune diseases and cancer, autoantibodies are expressed at altered levels relative to those observed in healthy individuals.

The term “protein chip” is used in the present application synonymously with protein array or microarray.

The phrase “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition, physical features (lumps or hard areas in or on tissue), or histological or biochemical analysis of biopsied or sampled tissue or cells, or a combination of these.

Similarly, a prognosis is often determined by examining one or more “prognostic indicators”, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability of having a disease or condition in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. For example, preferred prognostic markers can predict the onset of an autoimmune disease in a patient with one or more target antibodies of Table 1, or a more advanced stage of an autoimmune disease in a patient diagnosed with the disease.

The term “correlating,” as used herein in reference to the use of diagnostic and prognostic indicators, refers to comparing the presence or amount of the indicator in a patient to its presence or amount in persons known to suffer from, or known to be at risk of, a given condition; or in persons known to be free of a given condition. As discussed above, a marker level in a patient sample can be compared to a level known to be associated with autoimmune disease. The sample's marker level is said to have been correlated with a diagnosis; that is, the skilled artisan can use the marker level to determine whether the patient has an autoimmune disease, and respond accordingly. Alternatively, the sample's marker level can be compared to a marker level known to be associated with a good outcome (e.g., the absence of autoimmune disease, etc.). In preferred embodiments, a profile of marker levels are correlated to a global probability or a particular outcome using ROC curves.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

“Diagnostic” means identifying the presence or nature of a pathologic condition. Diagnostic methods differ in their sensitivity and specificity. While a particular diagnostic method may not provide a definitive diagnosis of a condition, it suffices if the method provides a positive indication that aids in diagnosis.

“Sensitivity” is defined as the percent of diseased individuals (individuals with autoimmune disease) in which the biomarker of interest is detected (true positive number/total number of diseased×100). Nondiseased individuals diagnosed by the test as diseased are “false positives”.

“Specificity” is defined as the percent of nondiseased individuals for which the biomarker of interest is not detected (true negative/total number without disease×100). Diseased individuals not detected by the assay are “false negatives.” Subjects who are not diseased and who test negative in the assay, are termed “true negatives.”

A “diagnostic amount” of a marker refers to an amount of a marker in a subject's sample that is consistent with a diagnosis of autoimmune disease. A diagnostic amount can be either in absolute amount (e.g., X nanogram/ml) or a relative amount (e.g. relative intensity of signals).

A “test amount” of a marker refers to an amount of a marker present in a sample being tested. A test amount can be either in absolute amount (e.g., X nanogram/ml) or a relative amount (e.g., relative intensity of signals).

A “control amount” of a marker can be any amount or a range of amount which is to be compared against a test amount of a marker. For example, a control amount of a marker can be the amount of a marker (e.g., seminal basic protein) in an autoimmune disease patient, or a normal patient. A control amount can be either in absolute amount (e.g., X nanogram/ml) or a relative amount (e.g., relative intensity of signals).

“Detect” refers to identifying the presence, absence or amount of the object to be detected.

“Label” or a “detectable moiety” refers to a composition detectable by spectroscopic, photochemical, biochemical, immunochemical, or chemical means. For example, useful labels include radiolabels such as ³²P, ³⁵S, or ¹²⁵I; fluorescent dyes; chromophores, electron-dense reagents; enzymes that generate a detectable signal (e.g., as commonly used in an ELISA); or spin labels. The label or detectable moiety has or generates a measurable signal, such as a radioactive, chromogenic, or fluorescent signal, that can be used to quantify the amount of bound detectable moiety in a sample. The detectable moiety can be incorporated in or attached to a primer or probe either covalently, or through ionic, van der Waals or hydrogen bonds, e.g., incorporation of radioactive nucleotides, or biotinylated nucleotides that are recognized by streptavidin. The label or detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules. (See, e.g., P. D. Fahrlander and A. Klausner, Bio/Technology 6:1165 (1988)). Quantitation of the signal is achieved by, e.g., scintillation counting, densitometry, or flow cytometry.

“Measure” in all of its grammatical forms, refers to detecting, quantifying or qualifying the amount (including molar amount), concentration or mass of a physical entity or chemical composition either in absolute terms in the case of quantifying, or in terms relative to a comparable physical entity or chemical composition.

“Immunoassay” is an assay in which an antibody specifically binds an antigen to provide for the detection and/or quantitation of the antibody or antigen. An immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Immune reactivity” as used herein means the presence or level of binding of an antibody or antibodies in a sample to one or more target antigens. A “pattern of immune reactivity” refers to the profile of binding of antibodies in a sample to a plurality of target antigens.

As used herein, “target antigen” refers to a protein, or to a portion, fragment, variant, isoform, processing product thereof having immunoreactivity of the protein, that is used to determine the presence, absence, or amount of an antibody in a sample from a subject. A “test antigen” is a protein evaluated for use as a target antigen. A test antigen is therefore a candidate target antigen, or a protein used to determine whether a portion of a test population has antibodies reactive against it. Use of the terms “target antigen”, “test antigen”, “autoantigen”, and, simply, “antigen” is meant to include the complete wild type mature protein, or can also denote a precursor, processed form (including, a proteolytically processed or otherwise cleaved form) unprocessed form, post-translationally modified, or chemically modified form of the protein indicated, in which the target antigen, test antigen, or antigen retains or possesses the specific binding characteristics of the referenced protein to one or more autoantibodies of a test sample. The protein can have, for example, one or more modifications not typically found in the protein produced by normal cells, including aberrant processing, cleavage or degradation, oxidation of amino acid residues, atypical glycosylation pattern, etc. The use of the terms “target antigen”, “test antigen”, “autoantigen”, or “antigen” also include splice isoforms or allelic variants of the referenced proteins, or can be sequence variants of the referenced protein, with the proviso that the “target antigen”, “test antigen”, “autoantigen”, or “antigen” retains or possesses the immunological reactivity of the referenced protein to one or more autoantibodies of a test sample. Use of the term “target antigen”, “test antigen”, “autoantigen”, or simply “antigen” specifically encompasses fragments of a referenced protein (“antigenic fragments”) that have the antibody binding specificity of the reference protein.

### Methods

The invention provides, in one aspect, a method of detecting one or more target antibodies in a test sample from an individual. The method includes: contacting the test sample from the individual with one or more target antigens of the invention, each comprising an autoantigen of Table 1, or a fragment thereof that includes an epitope recognized by a target antibody; and detecting binding of one or more antibodies in the sample to one or more target antigens, thereby detecting the presence of the one or more target antibodies in the sample. The target antigen can be any of the target antigens provided in Table 1, or a fragment thereof that includes an epitope. Furthermore, the target antigen can be a panel of target antigens that includes, for example, 2, 3, 4, 5, 6, 7, 8, 9, 10, 15, 20, 25, 50, or all target antigens of Table 1. The method can be carried out using virtually any immunoassay method. Non-limiting examples of immunoassay methods are provided below.

The individual from whom the test sample is taken can be any individual, healthy or suspected of having an autoimmune disease, and in some embodiments is an individual that is being screened for RA, SLE or ANCA.

Binding is typically detected using an immunoassay, which can be in various formats as described in detail below. Detection of binding in certain illustrative embodiments makes use of one or more solid supports to which the test antigen is immobilized on a substrate to which the sample from an individual, typically a human subject, is applied. After incubation of the sample with the immobilized antigen, or optionally, concurrently with the incubation of the sample, an antibody that is reactive against human antibodies (for example, an anti-human IgG antibody that is from a species other than human, for example, goat, rabbit, pig, mouse, etc.) can be applied to the solid support with which the sample is incubated. The non-human antibody is directly or indirectly labeled. After removing nonspecifically bound antibody, signal from the label that is significantly above background level is indicative of binding of a human antibody from the sample to a test antigen on the solid support.

In the methods provided herein, the sample can be any sample of cells or tissue, or of bodily fluid. Since the autoantibodies being screened for circulate in the blood and are fairly stable in blood sample, in certain illustrative embodiments, the test sample is blood or a fraction thereof, such as, for example, serum. The sample can be unprocessed prior to contact with the test antigen, or can be a sample that has undergone one or more processing steps. For example, a blood sample can be processed to remove red blood cells and obtain serum.

The test sample can be contacted with a test antigen provided in solution phase, or the test antigen can be provided bound to a solid support. In preferred embodiments, the detection is performed by an immunoassay, as described in more detail below. Detection of binding of the target sample to a test antigen indicates the presence of an autoantibody that specifically binds the test antigen in the sample. Identifying an autoantibody present in a sample from an individual can be used to identify biomarkers of a disease or condition, or to diagnose a disease or condition.

The detection can be performed on any solid support, such as a bead, dish, plate, well, sheet, membrane, slide, chip, or array, such as a protein array, which can be a microarray, and can optionally be a high density microarray.

The detection method can provide a positive/negative binding result, or can give a value that can be a relative or absolute value for the level of the autoantibody biomarker in the sample. The result can provide a diagnosis, prognosis, or be used as an indicator for conducting further tests or evaluation that may or may not result in a diagnosis or prognosis.

The method includes detecting more than one autoantibody in a sample from an individual, in which one or more of the test antigens used to detect autoantibodies is a test antigen of Table 1.

A fragment that includes an epitope recognized by an antibody can be at least 5, 6, 7, 8, 9, 10, 11, 12, 13, 14, 15, 20, 25, 30, 35, 40, 45, 50, 75, 100, 150, 200, 250, 300, 350, 400, 450, 500, 750, or 1000 amino acids in length. The fragment can also be between 5, 10, 15, 20, 25, 50, 75, 100, 150, 200, or 250 and one amino acid less than the entire length of an autoantigen. Typically, such epitopes are characterized in advance such that it is known that autoantibodies for a given autoantigen recognize the epitope. Methods for epitope mapping are well known in the art.

In some embodiments, the detection is performed on a protein array, which can be a microarray, and can optionally be a microarray that includes proteins at a concentration of at least 100/cm² or 1000/cm², or greater than 400/cm².

The detection method can provide a positive/negative binding result, or can give a value that can be a relative or absolute value for the level of the autoantibody biomarker in the sample.

The method can be repeated over time, for example, to monitor a pre-disease state, to monitor progression of a disease, or to monitor a treatment regime. The results of a diagnostic test that determines the immune reactivity of a patient sample to a test antigen can be compared with the results of the same diagnostic test done at an earlier time. Significant differences in immune reactivity over time can contribute to a diagnosis or prognosis of autoimmune disease.

In some preferred embodiments, the biomarker detection panel has an ROC/AUC of 0.550 or greater, of 0.600 or greater, 0.650 or greater, 0.700 or greater, 0.750 or greater, 0.800 or greater, 0.850 or greater, or 0.900 or greater for distinguishing between a normal state and a disease state in a subject.

A target antigen present in a biomarker detection panel can be an entire mature form of a protein, such as a protein referred to as a target antigen (for example, a target antigen listed in Table 1, Table 2, Table 3 or Table 5), or can be a precursor, processed form, unprocessed form, isoforms, variant, a fragment thereof that includes an epitope, or allelic variant thereof, providing that the modified, processed, or variant for of the protein has the ability to bind autoantigens present in samples from individuals.

In some embodiments, a biomarker detection panel used to detect autoimmune disease comprises one or more target antigens of Table 1. In some embodiments, a biomarker detection panel used to detect autoimmune disease comprises two or more target antigens of Table 1. In some embodiments, a biomarker detection panel used to detect autoimmune disease comprises three or more target antigens of Table 1. In some embodiments, a biomarker detection panel used to detect autoimmune disease comprises four or more target antigens of Table 1. In some embodiments, the test sample is contacted with a biomarker detection panel comprising five or more target antigens of Table 1. In some embodiments, the biomarker detection panel used in the methods of the invention includes six, seven, eight, nine, ten, eleven or twelve target antigens of Table 1. In some embodiments, the biomarker detection panel used in the methods of the invention includes 12, 13, 14, 15, 16, 17, 18, 19, 20, or more target antigens of Table 1. In some embodiments, the test sample is contacted with a biomarker detection panel comprising 21, 22, 23, 24, 25, 26, 27, 28, 29, or 30 antigens of Table 1. A biomarker detection panel can comprise between 30 and 35 antigens of Table 1, between 35 and 40 antigens of Table 1, between 40 and 45 antigens of Table 1, between 45 and 50 antigens of Table 1, between 50 and 55 antigens of Table 1, between 55 and 60 antigens of Table 1, between 60 and 65 antigens of Table 1, between 65 and 70 antigens of Table 1, between 70 and 75 antigens of Table 1, between 75 and 80 antigens of Table 1, between 80 and 85 antigens of Table 1, between 85 and 90 antigens of Table 1, between 90 and 95 antigens of Table 1, between 95 and 100 antigens of Table 1, between 100 and 105 antigens of Table 1, or between 105 and 108 antigens of Table 1. In all of the previous embodiments, one or more of the test antigens of Table 1 present in the biomarker detection panel can be a target antigen of Table 2, Table 3 or Table 5.

### Immunoassays

Virtually any immunoassay technique known in the art can be used to detect antibodies that bind an antigen according to methods and kits of the present invention. Such immunoassay methods include, without limitation, radioimmunoassays, immunohistochemistry assays, competitive-binding assays, Western Blot analyses, ELISA assays, sandwich assays, two-dimensional gel electrophoresis (2D electrophoresis) and non-gel based approaches such as mass spectrometry or protein interaction profiling, all known to those of ordinary skill in the art. These methods may be carried out in an automated manner, as is known in the art. Such immunoassay methods may also be used to detect the binding of antibodies in a sample to a target antigen.

In one example of an ELISA method, the method includes incubating a sample with a target protein and incubating the reaction product formed with a binding partner, such as a secondary antibody, that binds to the reaction product by binding to an antibody from the sample that associated with the target protein to form the reaction product. In some cases these may comprise two separate steps, in others, the two steps may be simultaneous, or performed in the same incubation step. Examples of methods of detection of the binding of the target protein to an antibody, is the use of an anti-human IgG (or other) antibody or protein A. This detection antibody may be linked to, for example, a peroxidase, such as horseradish peroxidase.

Using protein arrays for immunoassays allows the simultaneous analysis of multiple proteins. For example, target antigens or antibodies that recognize biomarkers that may be present in a sample are immobilized on microarrays. Then, the biomarker antibodies or proteins, if present in the sample, are captured on the cognate spots on the array by incubation of the sample with the microarray under conditions favoring specific antigen-antibody interactions. The binding of protein or antibody in the sample can then be determined using secondary antibodies or other binding labels, proteins, or analytes. Comparison of proteins or antibodies found in two or more different samples can be performed using any means known in the art. For example, a first sample can be analyzed in one array and a second sample analyzed in a second array that is a replica of the first array.

The term “sandwich assay” refers to an immunoassay where the antigen is sandwiched between two binding reagents, which are typically antibodies. The first binding reagent/antibody is attached to a surface and the second binding reagent/antibody comprises a detectable moiety or label. Examples of detectable moieties include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

A variety of different solid phase substrates can be used to detect a protein or antibody in a sample, or to quantitate or determine the concentration of a protein or antibody in a sample. The choice of substrate can be readily made by those of ordinary skill in the art, based on convenience, cost, skill, or other considerations. Useful substrates include without limitation: beads, bottles, surfaces, substrates, fibers, wires, framed structures, tubes, filaments, plates, sheets, and wells. These substrates can be made from: polystyrene, polypropylene, polycarbonate, glass, plastic, metal, alloy, cellulose, cellulose derivatives, nylon, coated surfaces, acrylamide or its derivatives and polymers thereof, agarose, or latex, or combinations thereof. This list is illustrative rather than exhaustive.

Other methods of protein detection and measurement described in the art can be used as well. For example, a single antibody can be coupled to beads or to a well in a microwell plate, and quantitated by immunoassay. In this assay format, a single protein can be detected in each assay. The assays can be repeated with antibodies to many analytes to arrive at essentially the same results as can be achieved using the methods of this invention. Bead assays can be multiplexed by employing a plurality of beads, each of which is uniquely labeled in some manner. For example each type of bead can contain a pre-selected amount of a fluorophore. Types of beads can be distinguished by determining the amount of fluorescence (and/or wavelength) emitted by a bead. Such fluorescently labeled beads are commercially available from Luminex Corporation (Austin, Tex.; see the worldwide web address of luminexcorp.com). The Luminex assay is very similar to a typical sandwich ELISA assay, but utilizes Luminex microspheres conjugated to antibodies or proteins (Vignali, J. Immunol. Methods 243:243-255 (2000)).

The methodology and steps of various antibody assays are known to those of ordinary skill in the art. Additional information may be found, for example, in Antibodies: A Laboratory Manual, Cold Spring Harbor Laboratory, Chap. 14 (1988); Bolton and Hunter, “Radioimmunoassay and Related Methods,” in Handbook of Experimental Immunology (D. M. Weir, ed.), Blackwell Scientific Publications, 1996; and Current Protocols in Immunology, (John E. Coligan, et al., eds) (1993).

The antibodies used to perform the foregoing assays can include polyclonal antibodies, monoclonal antibodies and fragments thereof as described supra. Monoclonal antibodies can be prepared according to established methods (see, e.g., Kohler and Milstein (1975) Nature 256:495; and Harlow and Lane (1988) Antibodies: A Laboratory Manual (C.H.S.P., N.Y.)).

An antibody can be a complete immunoglobulin or an antibody fragment. Antibody fragments used herein, typically are those that retain their ability to bind an antigen. Antibodies subtypes include IgG, IgM, IgA, IgE, or an isotype thereof (e.g., IgG1, IgG2a, IgG2b or IgG3). Antibody preparations can by polyclonal or monoclonal, and can be chimeric, humanized or bispecific versions of such antibodies. Antibody fragments include but are not limited to Fab, Fab′, F(ab)′2, Dab, Fv and single-chain Fv (ScFv) fragments. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. Antibodies sometimes are derivatized with a functional molecule, such as a detectable label (e.g., dye, fluorophore, radioisotope, light scattering agent (e.g., silver, gold)) or binding agent (e.g., biotin, streptavidin), for example.

In certain embodiments, one or more diagnostic (or prognostic) biomarkers, such as one or more autoantibody biomarkers, are correlated to a condition or disease by the presence or absence of the biomarker(s). In other embodiments, threshold level(s) of a diagnostic or prognostic biomarker(s) can be established, and the level of the biomarker(s) in a sample can simply be compared to the threshold level(s).

As will be understood, for any particular biomarker, a distribution of biomarker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a biomarker changes with the disease) the test is considered to be abnormal and below which the test is considered to be normal. Receiver Operating Characteristic curves, or “ROC” curves, are typically generated by plotting the value of a variable versus its relative frequency in “normal” and “disease” populations. The area under the ROC curve is a measure of the probability that the perceived measurement will allow correct identification of a condition. ROC curves can also be generated using relative, or ranked, results. Methods of generating ROC curves and their use are well known in the art. See, e.g., Hanley et al., Radiology 143: 29-36 (1982).

One or more test antigens may have relatively low diagnostic or prognostic value when considered alone, but when used as part of a panel that includes other reagents for biomarker detection (such as but not limited to other test antigens), such test antigens can contribute to making a particular diagnosis or prognosis. In preferred embodiments, particular threshold values for one or more test antigens in a biomarker detection panel are not relied upon to determine if a profile of marker levels obtained from a subject are indicative of a particular diagnosis or prognosis. Rather, the present invention may utilize an evaluation of the entire marker profile of a biomarker detection panel, for example by plotting ROC curves for the sensitivity of a particular biomarker detection panel. In these methods, a profile of biomarker measurements from a sample of an individual is considered together to provide an overall probability (expressed either as a numeric score or as a percentage risk) that an individual has an autoimmune disease, for example. In such embodiments, an increase in a certain subset of biomarkers (such as a subset of biomarkers that includes one or more autoantibodies) may be sufficient to indicate a particular diagnosis (or prognosis) in one patient, while an increase in a different subset of biomarkers (such as a subset of biomarkers that includes one or more autoantibodies) may be sufficient to indicate the same or a different diagnosis (or prognosis) in another patient. Weighting factors may also be applied to one or more biomarkers being detected. As one example, when a biomarker is of particularly high utility in identifying a particular diagnosis or prognosis, it may be weighted so that at a given level it alone is sufficient to indicate a positive diagnosis. In another example, a weighting factor may provide that no given level of a particular marker is sufficient to signal a positive result, but only signals a result when another marker also contributes to the analysis.

In preferred embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%.

Using various subsets of the test antigens provided in Table 1, the present invention provides test antibodies for detecting autoantibodies in a sample from an individual, antibodies for detecting autoimmune disease in an individual, and biomarker detection panels comprising combinations of the test antigens of Table 1 that can be used to detect and/or diagnose autoimmune disease, specifically RA, SLE and ANCA, with high sensitivity and specificity. Accordingly, methods, compositions, and kits are provided herein for the detection, diagnosis, staging, and monitoring of prostate cancer in individuals.

Automated systems for performing immunoassays, such as those utilized in the methods herein, are widely known and used in medical diagnostics. For example, random-mode or batch analyzer immunoassay systems can be used, as are known in the art. These can utilize magnetic particles or non-magnetic particles or microparticles and can utilize a fluorescence or chemiluminescence readout, for example. As non-limiting examples, the automated system can be an automated microarray hybridization station, an automated liquid handling robot, the Beckman ACCESS paramagnetic-particle, an chemiluminescent immunoassay, the Bayer ACS:180 chemiluminescent immunoassay or the Abbott AxSYM microparticle enzyme immunoassay. Such automated systems can be designed to perform methods provided herein for an individual antigen or for multiple antigens without multiple user interventions.

### Biomarker Detection Panels

The invention also provides biomarker detection panels for diagnosing, prognosing, monitoring, or staging autoimmune disease, in which the biomarker detection panels comprise two or more target antigens selected from Table 1, in which at least 50% of the proteins of the test panel are proteins of Table 1. In some preferred embodiments, the proteins of the biomarker detection panel are provided on one or more solid supports, in which at least 50% of the proteins on the one or more solid supports to which the proteins of the panel are bound are of Table 1. Proteins of a biomarker detection panel can be provided bound to a solid support in the form of a bead, matrix, dish, well, plate, slide, sheet, membrane, filter, fiber, chip, or array. In some preferred embodiments, the proteins of the biomarker detection panel are provided on a protein array in which 50% or more of the proteins on the array are target antigens of the biomarker detection panel.

The set of biomarkers in a biomarker detection panel are associated, either electronically, or preferably physically. For example, each biomarker of a biomarker detection panel can be provided in isolated form, in separate tubes that are sold and/or shipped together, for example as part of a kit. In certain embodiments, isolated biomarkers are formed into a detection panel by attaching them to the same solid support. The biomarkers of a biomarker panel can also be mixed together in the same solution.

The invention also provides biomarker detection panels for diagnosing, prognosing, monitoring, or staging autoimmune disease, in which the biomarker detection panels comprise 2, 3, 4, 5, 6, 7, 8, 9, 10, 15, 20, 25, 30, 35, 40, 45, 50 or more target antigens selected from Table 1, or in certain preferred embodiments, Table 2, Table 3 or Table 5, in which at least 55%, 60%, 65%, 70%, or 75% of the proteins of the test panel are proteins of Table 1, Table 2, Table 3 or Table 5 respectively. In some preferred embodiments, the proteins of the biomarker detection panel are provided on one or more solid supports, in which at least 55%, 60%, 65%, 70%, 75%, 80%, 85%, 90%, 95% or 100% of the proteins on the one or more solid supports to which the proteins of the panel are bound are of Table 1, Table 2, Table 3 or Table 5. In some preferred embodiments, the proteins of the biomarker detection panel are provided on a protein array in which at least 55%, 60%, 65%, 70%, or 75%, 80%, 85%, 90%, 95% or 100% of the proteins on the array are target antigens of the biomarker detection panel.

In some embodiments, the biomarker detection panel used in the methods of the invention includes 6, 7, 8, 9, 10, 11, or 12 target antigens of Table 1. In some embodiments, the biomarker detection panel used in the methods of the invention includes 13, 14, 15, 16, 17, 18, 19, 20, or more target antigens of Table 1. In some embodiments, the test sample is contacted with a biomarker detection panel comprising 21, 22, 23, 24, 25, 26, 27, 28, 29, or 30 antigens of Table 1. A biomarker detection panel can comprise between 30 and 35 antigens of Table 1, between 35 and 40 antigens of Table 1, between 40 and 45 antigens of Table 1, between 45 and 50 antigens of Table 1, between 50 and 55 antigens of Table 1, between 55 and 60 antigens of Table 1, between 60 and 65 antigens of Table 1, between 65 and 70 antigens of Table 1, between 70 and 75 antigens of Table 1, between 75 and 80 antigens of Table 1, between 80 and 85 antigens of Table 1, between 85 and 90 antigens of Table 1, between 90 and 95 antigens of Table 1, between 95 and 100 antigens of Table 1, between 100 and 105 antigens of Table 1, or between 105 and 108 antigens of Table 1.

Also included in the invention is a composition that comprises a biomarker detection panel for diagnosing, prognosing, monitoring, or staging autoimmune disease that comprises two or more target antigens selected from Table 1, in which at least one of the two or more target antigens is bound to an autoantibody from a sample of an individual. The invention also includes a biomarker detection panel for diagnosing, prognosing, monitoring, or staging autoimmune disease that comprises 3, 4, 5, 6, 7, 8, 9, 10, 11, 12, 13, 14, 15, 16, 17, 18, 19, 20, 21, 22, 23, 24, 25, 26, 27, 28, 29, 30 or more target antigens selected from Table 1, in which at least one of the two or more target antigens is bound to an autoantibody from a sample of an individual. Also included in the invention is a composition that comprises a biomarker detection panel for diagnosing, prognosing, monitoring, or staging autoimmune disease that comprises two or more target antigens selected from Table 2, Table 3 or Table 5, in which at least one of the target antigens of the array is bound to an autoantibody from a sample of an individual. The arrays having bound antibody from a sample can be arrays in which at least 55%, 60%, 65%, 70%, 75%, 80%, 85%, 90%, of 95% of the protein bound to the arrays are proteins of Table 1.

### Method for Synthesizing Protein Antigens

The methods, kits, and systems provided herein include autoantigens, which typically are protein antigens. To obtain protein antigens to be used in the methods provided herein, known methods can be used for making and isolating viral, prokaryotic or eukaryotic proteins in a readily scalable format, amenable to high-throughput analysis. For example, methods include synthesizing and purifying proteins in an array format compatible with automation technologies. Therefore, in one embodiment, protein microarrays for the invention a method for making and isolating eukaryotic proteins comprising the steps of growing a eukaryotic cell transformed with a vector having a heterologous sequence operatively linked to a regulatory sequence, contacting the regulatory sequence with an inducer that enhances expression of a protein encoded by the heterologous sequence, lysing the cell, contacting the protein with a binding agent such that a complex between the protein and binding agent is formed, isolating the complex from cellular debris, and isolating the protein from the complex, wherein each step is conducted in a 96-well format.

In a particular embodiment, eukaryotic proteins are made and purified in a 96-array format (i.e., each site on the solid support where processing occurs is one of 96 sites), e.g., in a 96-well microtiter plate. In another embodiment, the solid support does not bind proteins (e.g., a non-protein-binding microtiter plate).

In certain embodiments, proteins are synthesized by in vitro translation according to methods commonly known in the art. For example, proteins can be expressed using a wheat germ, rabbit reticulocyte, or bacterial extract, such as the Expressway.

Any expression construct having an inducible promoter to drive protein synthesis can be used in accordance with the methods of the invention. The expression construct may be, for example, tailored to the cell type to be used for transformation. Compatibility between expression constructs and host cells are known in the art, and use of variants thereof are also encompassed by the invention.

In a particular embodiment, the fusion proteins have GST tags and are affinity purified by contacting the proteins with glutathione beads. In further embodiment, the glutathione beads, with fusion proteins attached, can be washed in a 96-well box without using a filter plate to ease handling of the samples and prevent cross contamination of the samples.

In addition, fusion proteins can be eluted from the binding compound (e.g., glutathione bead) with elution buffer to provide a desired protein concentration. In a specific embodiment, fusion proteins are eluted from the glutathione beads with 30 μl of elution buffer to provide a desired protein concentration.

For purified proteins that will eventually be spotted onto microscope slides, the glutathione beads are separated from the purified proteins. In one example, all of the glutathione beads are removed to avoid blocking of the microarrays pins used to spot the purified proteins onto a solid support. In one embodiment, the glutathione beads are separated from the purified proteins using a filter plate, for example, comprising a non-protein-binding solid support. Filtration of the eluate containing the purified proteins should result in greater than 90% recovery of the proteins.

The elution buffer may, for example, comprise a liquid of high viscosity such as, for example, 15% to 50% glycerol, for example, about 25% glycerol. The glycerol solution stabilizes the proteins in solution, and prevents dehydration of the protein solution during the printing step using a microarrayer.

Purified proteins may, for example, be stored in a medium that stabilizes the proteins and prevents desiccation of the sample. For example, purified proteins can be stored in a liquid of high viscosity such as, for example, 15% to 50% glycerol, for example, in about 25% glycerol. In one example, samples may be aliquoted containing the purified proteins, so as to avoid loss of protein activity caused by freeze/thaw cycles.

The skilled artisan can appreciate that the purification protocol can be adjusted to control the level of protein purity desired. In some instances, isolation of molecules that associate with the protein of interest is desired. For example, dimers, trimers, or higher order homotypic or heterotypic complexes comprising an overproduced protein of interest can be isolated using the purification methods provided herein, or modifications thereof. Furthermore, associated molecules can be individually isolated and identified using methods known in the art (e.g., mass spectroscopy).

The protein antigens once produced can be used in the biomarker panels, methods and kits provided herein as part of a “positionally addressable” array. The array includes a plurality of target antigens, with each target antigen being at a different position on a solid support. The array can include, for example 1, 2, 3, 4, 5, 8, 9, 10, 11, 12, 13, 14, 15, 16, 17, 18, 19, 20, 21, 22, 23, 24, 25, 26, 27, 28, 29, 30, 40, 50, 100, 200, 300, 400, or 500 different proteins. The array can include 1, 2, 3, 4, 5, 10, 15, 20, 25, 50, 100 or all the proteins of Table 1. In one aspect, the majority of proteins on an array include proteins identified as autoantigens that can have diagnostic value for a particular disease or medical condition when provided together autoantigen biomarker detection panel.

In one aspect, the protein array is a bead-based array. In another aspect, the protein array is a planar array. Methods for making protein arrays, such as by contact printing, are well known. In some embodiments, the detection is performed on a protein array, which can be a microarray, and can optionally be a microarray that includes proteins at a concentration of at least 100/cm² or 1000/cm², or greater than 400/cm².

### Kits

In certain embodiments of the invention, kits are provided. Thus, in some embodiments, a kit is provided that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30-34, 35-39, 40-44, 45-49, 50-54, 55-59, 60-64, 65-69, 70-74, 75-79, 80-84, 85-89, 90-94, 95-100, 100-105, or 106-108 of the test antigen proteins provided in Table 1. In certain aspects the kit includes up to 10, 50, 100, or 108 of the test antigen proteins of Table 1. A kit of the invention can include any of the biomarker detection panels disclosed herein, including, but not limited to, a biomarker panel comprising two or more test antigens of Table 1, and a biomarker panel comprising two or more test antigens of Table 2, Table 3, or Table 5.

In one embodiment, a kit for diagnosing an autoimmune disease comprises one or more, two or more, ten or more, twenty or more, fifty or more, or all of the autoantigens of Table 1 or a fragment thereof comprising an epitope; and means for detecting if one or more molecules in a test sample binds to one or more of the antigens. In some embodiments, the kits and protein arrays of the present invention contain less than 1,000 polypeptides, or less than 100 polypeptides. In a further embodiment, the kit further comprises a control antibody against one or more of the antigens.

In a further embodiment, the kit comprises one or more, two or more, ten or more, twenty or more, fifty or more, or all of the antigens selected from the group comprising of Table 2 or fragments thereof comprising an epitope. In a related embodiment, the kit consists essentially of one or more, two or more, ten or more, twenty or more, fifty or more, or all of the antigens selected from the group comprising of Table 2 or fragments thereof comprising an epitope.

In another embodiment, the kit comprises one or more, two or more, ten or more, twenty or more, fifty or more, or all of the antigens selected from the group comprising of Table 3 or fragments thereof comprising an epitope. In a related embodiment, the kit consists essentially of one or more, two or more, ten or more, twenty or more, fifty or more, or all of the antigens selected from the group comprising of Table 3 or fragments thereof comprising an epitope.

In another embodiment, the kit comprises one or more, two or more, ten or more, twenty or more, fifty or more, or all of the antigens selected from the group comprising of Table 5 or fragments thereof comprising an epitope. In a related embodiment, the kit consists essentially of one or more, two or more, ten or more, twenty or more, fifty or more, or all of the antigens selected from the group comprising of Table 5 or fragments thereof comprising an epitope.

In another embodiment, the kit comprises one or more, two or more, ten or more, twenty or more, fifty or more, or all of the antigens selected from the group comprising of Table 2 or fragments thereof comprising an epitope, in combination with one or more, two or more, ten or more, twenty or more, fifty or more, or all of the antigens selected from the group comprising of Table 3 or fragments thereof comprising an epitope.

In another embodiment, the kit comprises one or more, two or more, ten or more, twenty or more, fifty or more, or all of the antigens selected from the group comprising of Table 2 or fragments thereof comprising an epitope, in combination with one or more, two or more, ten or more, twenty or more, fifty or more, or all of the antigens selected from the group comprising of Table 5 or fragments thereof comprising an epitope.

In another embodiment, the kit comprises one or more, two or more, ten or more, twenty or more, fifty or more, or all of the antigens selected from the group comprising of Table 3 or fragments thereof comprising an epitope, in combination with one or more, two or more, ten or more, twenty or more, fifty or more, or all of the antigens selected from the group comprising of Table 5 or fragments thereof comprising an epitope.

The kit can include one or more positive controls, one or more negative controls, and/or one or more normalization controls.

The proteins of the kit may, for example, be immobilized on a solid support or surface. The proteins may, for example, be immobilized in an array. The protein microarray may use bead technology, such as the Luminex technology (Luminex Corp., Austin, Tex.). The test protein array may or may not be a high-density protein microarray that includes at least 100 proteins/cm². The kit can provide a biomarker detection panel of proteins as described herein immobilized on an array. At least 50%, at least 55%, at least 60%, at least 65%, at least 70%, at least 75%, at least 80%, at least 85%, at least 90%, or at least 95% of the proteins immobilized on the array can be proteins of the biomarker test pane. The array can include immobilized on the array one or more positive control proteins, one or more negative controls, and/or one or more normalization controls.

A kit may further comprise a reporter reagent to detect binding of human antibody to the proteins, such as, for example, an antibody that binds to human antibody, linked to a detectable label. A kit may further comprise reagents useful for various immune reactivity assays, such as ELISA, or other immunoassay techniques known to those of skill in the art. The assays in which the kit reagents can be used may be competitive assays, sandwich assays, and the label may be selected from the group of well-known labels used for radioimmunoassay, fluorescent or chemiluminescence immunoassay.

A kit can include reagents described herein in any combination. For example, in one aspect, the kit includes a biomarker detection panel as provided herein immobilized on a solid support and anti-human antibodies for detection in solution. The detection antibodies can comprise labels.

The kit can also include a program in computer readable form to analyze results of methods performed using the kits to practice the methods provided herein.

The kits of the present invention may also comprise one or more of the components in any number of separate containers, packets, tubes, vials, microtiter plates and the like, or the components may be combined in various combinations in such containers.

The kits of the present invention may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

### EXAMPLES

The examples set forth below illustrate, but do not limit the invention.

### Example 1

Serum from ten healthy control individuals, twelve individuals with RA prior to and following initiation of Remicade® treatment, twenty individuals with SLE, and twenty individuals with ANCA were profiled against a high throughput human protein array. Serum samples were diluted 1:150 and used to probe human ProtoArray™. Specifically, arrays were blocked for 1 hour, incubated with dilute serum solution for 90 minutes, washed 3×10 minutes, incubated with anti-human IgG antibody conjugated to AlexaFluor 647 for 90 minutes, washed as above, dried, and scanned. Following scanning, data was acquired using specialized software. Background-subtracted signals from each population were normalized utilizing a quantile normalization strategy. All possible pairwise comparisons were performed between all groups of samples included in the study utilizing an M-statistics algorithm in which the M-statistic is identified that is associated with the lowest possible p-value for a particular pairwise comparison of sample populations.

Proteins of interest identified as significant interactors with antibodies present in the serum from autoimmune disease patients included a number of known autoantigens including proteinase-3, myeloperoxidase, CCP peptide, and ssDNA, as well as a number of candidate novel autoantigens. These autoantigens are listed in Table 1 and are further classified according to the corresponding autoimmune disease: RA (Table 2), SLE (Table 3), and ANCA (Table 5). Pairwise comparisons performed between RA at various timepoint pre- and post-Remicade® treatment identified a number of known and novel autoantigens for which either an increased or decreased autoantibody response is observed over the treatment timecourse as described above (Tables 7A and 7B).

### Example 2

Serum samples from healthy individuals as well as individuals with autoimmune diseases including RA (Rheumatoid Arthritis), SLE (Systemic Lupus Erythrematosus) and ANCA (Anti-Neutrophil Cytoplasmic Antibody) were profiled on ProtoArray™ human protein microarrays as described in Example 1. Utilizing the calculations as described below, a number of potential antigen biomarkers were identified for autoimmune diseases. These proteins have the potential to serve as important diagnostic or prognostic indicators. Instead of an assay containing thousands or tens of thousands of proteins, a test sample can be profiled against an assay containing just the antigens associated with autoimmune disease, or a specific autoimmune disease. The tables below identify the autoantigens for RA, SLE, and ANCA.

Tables 1-7 identify antigens according to Genbank ID number for the nucleotide sequence that encodes the antigens. It is understood that an antigen of Tables 1-7 refers to a protein or fragments thereof that is encoded by the nucleotide sequence associated with the nucleotide ID number. Table 1 lists autoantigens associated with RA, SLE and ANCA. The autoantigens in Tables 2, 3 and 5 separately list the autoantigens associated with RA, SLE and ANCA, respectively, and are each a subset of the autoantigens of Table 1.

Table 1 is a list of autoantigens that were bound more often by antibodies from sera from RA, SLE and ANCA individuals than by antibodies from healthy individuals.

TABLE 1
Autoimmune disease patients vs. healthy patients

| Genbank ID | | | | |
|---|---|---|---|---|
| number of | | RA, | | |
| nucleic acid | | SLE or | | |
| coding for | Normal | ANCA | | |
| the protein | Count | Count | p-value | Name or description |
| BC000052.1 | 0 | 9 | 0.0117396 | Similar to peroxisome proliferative activated |
| | | | | receptor, alpha |
| BC000103.1 | 3 | 17 | 0.0048444 | NCK adaptor protein 2 |
| BC000175.2 | 1 | 12 | 0.0111660 | Hermansky-Pudlak syndrome 1, transcript variant 3 |
| BC000381.2 | 1 | 13 | 0.0055970 | TBP-like 1, mRNA |
| BC000442.1 | 1 | 13 | 0.0055972 | serine/threonine kinase 12 |
| BC000809.1 | 2 | 7 | 0.0185217 | transcription elongation factor A (SII)-like 1 |
| BC000914.1 | 1 | 15 | 0.0010990 | splicing factor, arginine/serine-rich 3 |
| BC000914.1 | 0 | 12 | 0.0014564 | splicing factor, arginine/serine-rich 3 |
| BC000997.2 | 1 | 12 | 0.0111660 | splicing factor, arginine/serine-rich 7, 35 kDa |
| BC001120.1 | 10 | 11 | 0.0117396 | lectin, galactoside-binding, soluble, 3 (galectin 3) |
| BC001371.2 | 0 | 9 | 0.0117396 | chromosome 20 open reading frame 31, mRNA |
| BC001396.1 | 3 | 17 | 0.0048440 | AD-003 protein |
| BC001662.1 | 0 | 9 | 0.0117396 | mitogen-activated protein kinase-activated protein |
| | | | | kinase 3 |
| BC001662.1 | 1 | 6 | 0.0173851 | mitogen-activated protein kinase-activated protein |
| | | | | kinase 3 |
| BC002637.1 | 0 | 5 | 0.0108359 | tribbles homolog 2 |
| BC002733.2 | 1 | 12 | 0.0111660 | mRNA, complete cds. |
| BC002880.1 | 1 | 14 | 0.0025987 | cysteinyl-tRNA synthetase |
| BC003168.1 | 0 | 9 | 0.0117396 | oxysterol binding protein-like 10, |
| BC004514.1 | 1 | 12 | 0.0111658 | hypothetical protein FLJ12584 |
| BC004514.1 | 1 | 7 | 0.0048821 | hypothetical protein FLJ12584 |
| BC005248.1 | 0 | 11 | 0.0030750 | eukaryotic translation initiation factor 1A, Y-linked |
| BC005332.1 | 0 | 11 | 0.0030747 | cDNA clone MGC: 12418 IMAGE: 3934658, |
| | | | | complete cds |
| BC006105.1 | 2 | 14 | 0.0130935 | chromosome 6 open reading frame 134, mRNA |
| BC006376.1 | 0 | 10 | 0.0061490 | N-myristoyltransferase 2 |
| BC006456.1 | 10 | 11 | 0.0117400 | KIAA0592 protein |
| BC006793.1 | 0 | 10 | 0.0061490 | GATA binding protein 3 |
| BC007228.1 | 0 | 9 | 0.0117400 | Taxol resistant associated protein 3 (TRAG-3) |
| BC007411.2 | 2 | 15 | 0.0062397 | diaphanous homolog 1 (Drosophila) |
| BC007411.2 | 2 | 7 | 0.0185217 | diaphanous homolog 1 (Drosophila) |
| BC007833.2 | 0 | 9 | 0.0117400 | phosphatidylinositol-4-phosphate 5-kinase, type I, |
| | | | | alpha, mRNA |
| BC007863.1 | 0 | 5 | 0.0108359 | platelet-activating factor acetylhydrolase, isoform Ib, |
| | | | | gamma subunit (29 kD) |
| BC007888.1 | 2 | 14 | 0.0130930 | eukaryotic translation initiation factor 2, subunit 2 |
| | | | | (beta, 38 kD) |
| BC007949.1 | 0 | 9 | 0.0117396 | eukaryotic translation elongation factor 1 gamma |
| BC008623.1 | 0 | 12 | 0.0014560 | hypothetical protein FLJ21044 similar to Rbig1, |
| | | | | cloneMGC: 16823 IMAGE: 4177689, |
| | | | | mRNA, complete cds. |
| BC009623.1 | 0 | 9 | 0.0117400 | nucleophosmin (nucleolar phosphoprotein B23, |
| | | | | numatrin) |
| BC009762.2 | 0 | 9 | 0.0117400 | mRNA, complete cds. |
| BC009873.1 | NA | NA | NA | clone MGC: 16442 IMAGE: 3946787 |
| BC010642.1 | 0 | 9 | 0.0117400 | zinc finger protein 22 (KOX 15), |
| BC011379.1 | 2 | 15 | 0.0062400 | DKFZP434H132 protein |
| BC011498.1 | 4 | 18 | 0.0072350 | Unknown (protein for MGC: 17017) |
| BC011668.1 | 1 | 12 | 0.0111660 | Similar to casein kinase 2, alpha 1 polypeptide |
| BC011707.1 | 10 | 11 | 0.0117400 | nuclear receptor binding factor 2, mRNA |
| BC011804.2 | 4 | 17 | 0.0183900 | chromosome 1 open reading frame 165, mRNA |
| BC011863.2 | 2 | 7 | 0.0185217 | Unknown (protein for MGC: 20604) |
| BC012105.1 | 3 | 8 | 0.0148845 | nuclear VCP-like, mRNA |
| BC012120.1 | 0 | 9 | 0.0117400 | nuclear factor I/C (CCAAT-binding transcription |
| | | | | factor) |
| BC012472.1 | 1 | 12 | 0.0111660 | ubiquitin D, mRNA |
| BC012876.1 | 0 | 14 | 0.0002665 | clone MGC: 17259 IMAGE: 4149333 |
| BC012924.1 | NA | NA | NA | dual adaptor of phosphotyrosine and 3- |
| | | | | phosphoinositides |
| BC013073.1 | 0 | 10 | 0.0061490 | chromosome 1 open reading frame 37, mRNA |
| BC013103.1 | 1 | 6 | 0.0173851 | Similar to hypothetical protein FLJ20435, |
| | | | | cloneMGC: 16997 IMAGE: 4343882, mRNAcomplete |
| | | | | cds. |
| BC013171.1 | 10 | 11 | 0.0117396 | cDNA clone MGC: 17065 IMAGE: 4344401, |
| | | | | complete cds |
| BC013567.1 | 10 | 11 | 0.0117400 | hypothetical protein FLJ11328 |
| BC014271.2 | 1 | 14 | 0.0025987 | endoglin (Osler-Rendu-Weber syndrome 1), mRNA |
| BC014435.1 | 0 | 5 | 0.0108359 | Unknown (protein for MGC: 22922) |
| BC014452.1 | 4 | 17 | 0.0183900 | cDNA clone IMAGE: 4903661 |
| BC014975.1 | 2 | 7 | 0.0185217 | hypothetical protein FLJ14668, mRNA |
| BC014991.1 | 0 | 9 | 0.0117396 | N-methylpurine-DNA glycosylase |
| BC015008.1 | 4 | 17 | 0.0183900 | hydroxyacylglutathione hydrolase-like, mRNA |
| BC015497.1 | 1 | 12 | 0.0111660 | cDNA clone MGC: 9014 IMAGE: 3913870, complete |
| | | | | cds |
| BC015715.1 | 1 | 12 | 0.0111660 | makorin, ring finger protein, 2 |
| BC015833.1 | 4 | 17 | 0.0183901 | cDNA clone MGC: 27152 IMAGE: 4691630, |
| | | | | complete cds |
| BC016057.1 | 10 | 11 | 0.0117396 | Usher syndrome 1C (autosomal recessive, severe), |
| | | | | mRNA |
| BC016312.1 | 10 | 11 | 0.0117396 | chromosome 15 open reading frame 15, mRNA |
| BC016380.1 | 1 | 14 | 0.0025987 | cDNA clone MGC: 27376 IMAGE: 4688477, |
| | | | | complete cds |
| BC016381.1 | 0 | 13 | 0.0006473 | cDNA clone MGC: 27378 IMAGE: 4688865, |
| | | | | complete cds |
| BC016381.1 | 1 | 6 | 0.0173851 | cDNA clone MGC: 27378 IMAGE: 4688865, |
| | | | | complete cds |
| BC016764.1 | 4 | 20 | 0.0003540 | ribulose-5-phosphate-3-epimerase, transcript |
| | | | | variant 1 |
| BC016764.1 | 3 | 16 | 0.0116173 | ribulose-5-phosphate-3-epimerase, transcript |
| | | | | variant 1 |
| BC016778.1 | 2 | 14 | 0.0130930 | HIV-1 rev binding protein 2, mRNA |
| BC016842.1 | 1 | 12 | 0.0111660 | family with sequence similarity 61, member A, |
| | | | | mRNA |
| BC017114.1 | 0 | 10 | 0.0061490 | hypothetical protein FLJ22833 |
| BC017865.1 | 0 | 11 | 0.0030747 | Fc fragment of IgG, low affinity IIIa, receptor |
| | | | | (CD16a), mRNA |
| BC018142.1 | 3 | 8 | 0.0148845 | caspase recruitment domain family, member 14, |
| | | | | mRNA |
| BC018302.1 | 0 | 9 | 0.0117396 | TRM1 tRNA methyltransferase 1 homolog (S. cerevisiae), |
| | | | | mRNA |
| BC018302.1 | 0 | 6 | 0.0030960 | TRM1 tRNA methyltransferase 1 homolog (S. cerevisiae), |
| | | | | mRNA |
| BC019337.1 | 10 | 11 | 0.0117400 | immunoglobulin heavy constant gamma 1 (G1m |
| | | | | marker), mRNA |
| BC019337.1 | 4 | 19 | 0.0021220 | immunoglobulin heavy constant gamma 1 (G1m |
| | | | | marker), mRNA |
| BC020622.1 | 1 | 13 | 0.0055972 | zinc finger, A20 domain containing 1, mRNA, |
| | | | | complete cds. |
| BC020647.1 | 2 | 14 | 0.0130930 | HSPC128 protein, mRNA |
| BC020962.1 | 0 | 9 | 0.0117396 | similar to glucosamine-6-sulfatases |
| BC022098.1 | 0 | 10 | 0.0061493 | cDNA clone MGC: 31944 IMAGE: 4878869, |
| | | | | complete cds |
| BC022231.1 | 1 | 12 | 0.0111660 | Ets2 repressor factor, mRNA |
| BC022325.1 | 0 | 16 | 0.0000333 | hypothetical protein FLJ12729 |
| BC022362.1 | 1 | 14 | 0.0025987 | cDNA clone MGC: 23888 IMAGE: 4704496, |
| | | | | complete cds |
| BC023569.1 | 0 | 9 | 0.0117400 | UPF3 regulator of nonsense transcripts homolog A |
| | | | | (yeast), transcript variant 2 |
| BC024289.1 | 0 | 10 | 0.0061493 | cDNA clone MGC: 39273 IMAGE: 5440834, |
| | | | | complete cds |
| BC024289.1 | 1 | 7 | 0.0048821 | cDNA clone MGC: 39273 IMAGE: 5440834, |
| | | | | complete cds |
| BC025314.1 | 3 | 17 | 0.0048444 | immunoglobulin heavy constant gamma 1 (G1m |
| | | | | marker), mRNA |
| BC025314.1 | 1 | 6 | 0.0173851 | immunoglobulin heavy constant gamma 1 (G1m |
| | | | | marker), mRNA |
| BC025345.1 | 4 | 19 | 0.0021220 | mRNA similar to LOC149651 (cDNA clone |
| | | | | MGC: 39393 IMAGE: 4862156), complete cds |
| BC025996.2 | 0 | 13 | 0.0006470 | cDNA clone MGC: 26787 IMAGE: 4838986 |
| BC027607.1 | 1 | 12 | 0.0111660 | clone MGC: 26892 IMAGE: 4828241 |
| BC028039.1 | 1 | 6 | 0.0173851 | hypothetical protein MGC39900 |
| BC028151.1 | 0 | 9 | 0.0117400 | DNA segment on chromosome X and Y (unique) |
| | | | | 155 expressed sequence, mRNA |
| BC028237.1 | 1 | 12 | 0.0111660 | growth differentiation factor 10, mRNA |
| BC028301.1 | 0 | 12 | 0.0014560 | mRNA similar to LOC147447 |
| BC029046.1 | 4 | 17 | 0.0183900 | H1 histone family, member 0, mRNA |
| BC029444.1 | 0 | 11 | 0.0030747 | cDNA clone MGC: 32714 IMAGE: 4692138, |
| | | | | complete cds |
| BC029609.1 | 0 | 10 | 0.0061493 | cDNA clone MGC: 39831 IMAGE: 5302675 |
| BC029827.1 | 0 | 9 | 0.0117400 | Down syndrome critical region gene 9, mRNA |
| BC029891.1 | 0 | 10 | 0.0061490 | transcription factor EC, mRNA |
| BC030219.1 | 1 | 14 | 0.0025990 | RAD51-like 1 (S. cerevisiae) |
| BC030219.1 | 1 | 6 | 0.0173851 | RAD51-like 1 (S. cerevisiae) |
| BC030590.1 | 1 | 12 | 0.0111658 | retinoblastoma binding protein 8, mRNA |
| BC030702.1 | 0 | 10 | 0.0061490 | hypothetical protein FLJ12847 |
| BC030814.1 | 0 | 14 | 0.0002665 | immunoglobulin kappa variable 1-5, mRNA |
| BC030983.1 | 2 | 17 | 0.0009742 | immunoglobulin lambda constant 1 (Mcg marker), |
| | | | | mRNA |
| BC030984.1 | 2 | 19 | 0.0000636 | cDNA clone MGC: 32654 IMAGE: 4701898, |
| | | | | complete cds |
| BC031074.1 | 1 | 16 | 0.0004141 | poly (ADP-ribose) polymerase family, member 16, |
| | | | | mRNA |
| BC032124.1 | 1 | 6 | 0.0173851 | bromodomain containing 3 |
| BC032334.1 | 0 | 5 | 0.0108359 | putative homeodomain transcription factor 2, |
| | | | | mRNA, complete cds. |
| BC032452.1 | 10 | 11 | 0.0117400 | immunoglobulin lambda constant 1 (Mcg marker), |
| | | | | mRNA |
| BC032462.1 | 0 | 10 | 0.0061490 | vacuolar protein sorting 29 (yeast), mRNA |
| BC032485.1 | 0 | 9 | 0.0117400 | hypothetical protein FLJ30473, |
| BC032485.1 | 1 | 16 | 0.0004141 | hypothetical protein FLJ30473 |
| BC032852.2 | 4 | 18 | 0.0072350 | melanoma antigen family B, 4, mRNA |
| BC032866.2 | 1 | 13 | 0.0055972 | eukaryotic translation initiation factor 5, transcript |
| | | | | variant 2, mRNA |
| BC033195.1 | 1 | 6 | 0.0173851 | leukocyte receptor cluster (LRC) member 12 |
| BC033856.1 | 3 | 17 | 0.0048440 | Similar to RIKEN cDNA 3110040D16 gene, |
| | | | | cloneMGC: 45395 IMAGE: 5123380, |
| | | | | mRNA, completecds. |
| BC034401.1 | 10 | 11 | 0.0117400 | Similar to LOC161981 |
| BC034954.2 | 2 | 7 | 0.0185217 | nucleosome assembly protein 1-like 3, mRNA |
| BC035314.1 | 4 | 17 | 0.0183900 | brix domain containing 1 |
| BC035568.1 | 0 | 9 | 0.0117400 | acylphosphatase 1, erythrocyte (common) type |
| BC036075.1 | 0 | 9 | 0.0117396 | GIPC PDZ domain containing family, member 2, |
| | | | | mRNA |
| BC036723.1 | 1 | 12 | 0.0111658 | Fc fragment of IgG, low affinity IIIa, receptor |
| | | | | (CD16a), mRNA |
| BC037906.1 | 2 | 7 | 0.0185217 | hypothetical protein FLJ11017, mRNA |
| BC038105.2 | 1 | 14 | 0.0025990 | membrane protein, palmitoylated 7 (MAGUK p55 |
| | | | | subfamily member 7) |
| BC039814.1 | 1 | 17 | 0.0001340 | zinc finger protein 265, transcript variant 2, mRNA |
| BC040844.1 | 1 | 14 | 0.0025990 | synaptotagmin binding, cytoplasmic RNA interacting |
| | | | | protein, mRNA |
| BC041037.1 | 0 | 7 | 0.0007145 | immunoglobulin heavy constant mu, mRNA |
| BC041157.1 | 0 | 11 | 0.0030747 | thromboxane A synthase 1 (platelet, cytochrome |
| | | | | P450, family 5, subfamily A), transcript variantTXS-I, |
| | | | | mRNA |
| BC042625.1 | 1 | 13 | 0.0055970 | LUC7-like 2 (S. cerevisiae), mRNA |
| BC044584.1 | 0 | 11 | 0.0030747 | DnaJ (Hsp40) homolog, subfamily C, member 4, |
| | | | | mRNA |
| BC050428.1 | 5 | 19 | 0.0088420 | katanin p60 (ATPase-containing) subunit A 1, |
| | | | | mRNA |
| BC051301.1 | 0 | 10 | 0.0061490 | TEA domain family member 2, mRNA |
| BC052806.1 | 0 | 9 | 0.0117400 | cDNA clone MGC: 61802 IMAGE: 5730155 |
| BC053656.1 | 10 | 11 | 0.0117400 | EGF-like repeats and discoidin I-like domains 3, |
| | | | | mRNA |
| BC053656.1 | 1 | 17 | 0.0001340 | EGF-like repeats and discoidin I-like domains 3, |
| | | | | mRNA |
| BC053664.1 | 0 | 12 | 0.0014564 | complete cds. |
| BC053866.1 | 0 | 9 | 0.0117400 | endothelin 3, transcript variant 2 |
| BC053872.1 | 0 | 9 | 0.0117400 | copine V, mRNA |
| BC053984.1 | 3 | 18 | 0.0016572 | cDNA clone MGC: 59926 IMAGE: 5480266, |
| | | | | complete cds |
| BC054034.1 | 1 | 12 | 0.0111660 | U11/U12 snRNP 35K, transcript variant 2 |
| BC055314.1 | 0 | 10 | 0.0061490 | C2f protein |
| BC056256.1 | 1 | 16 | 0.0004141 | immunoglobulin kappa constant, mRNA |
| BC057774.1 | 1 | 12 | 0.0111660 | hypothetical protein FLJ31455, mRNA |
| BC058903.1 | 0 | 9 | 0.0117400 | intercellular adhesion molecule 3, mRNA |
| BC063275.1 | 1 | 15 | 0.0010990 | eukaryotic translation initiation factor 2C, 1, mRNA |
| BC063479.1 | 1 | 15 | 0.0010990 | La ribonucleoprotein domain family, member 4, |
| | | | | mRNA |
| BC066938.1 | 4 | 17 | 0.0183901 | DEAD (Asp-Glu-Ala-Asp) box polypeptide 43, |
| | | | | mRNA |
| BC066987.1 | 0 | 9 | 0.0117396 | cDNA clone MGC: 87634 IMAGE: 4838596, |
| | | | | complete cds |
| BC067446.1 | 0 | 9 | 0.0117400 | disabled homolog 1 (Drosophila), mRNA |
| CTL1093 | 6 | 20 | 0.0076628 | Human IgG |
| CTL1094 | 10 | 11 | 0.0117400 | Influenza A |
| CTL2110 | 0 | 11 | 0.0030750 | DNA TOPOISIMERASE(Scl-70) |
| CTL2112 | 3 | 17 | 0.0048440 | ssDNA |
| CTL2130 | 1 | 18 | 0.0000351 | proteinase-3 |
| CTL2132 | 1 | 12 | 0.0111660 | myeloperoxidase |
| CTL2132 | 1 | 6 | 0.0173851 | myeloperoxidase_100 ug/ml_S |
| CTL2136 | 1 | 13 | 0.0055970 | U1-snRNP 68 PROTEIN |
| CTL2137 | 1 | 15 | 0.0010995 | La/SS-B (La) |
| CTL2138 | 0 | 14 | 0.0002670 | RNP COMPLEX |
| CTL2142 | 4 | 19 | 0.0021220 | ssDNA |
| CTL2145 | 0 | 14 | 0.0002670 | RIBOSOMAL RNA |
| CTL2152 | 2 | 14 | 0.0130930 | RNA POLYMERASE |
| NM_000997. | 0 | 9 | 0.0117400 | ribosomal protein L37 (RPL37) |
| NM_001014.2 | 1 | 14 | 0.0025990 | ribosomal protein S10 (RPS10) |
| NM_001015.2 | 10 | 11 | 0.0117396 | ribosomal protein S11 (RPS11) |
| NM_001106.2 | 1 | 12 | 0.0111660 | activin A receptor, type IIB (ACVR2B) |
| NM_001124.1 | 0 | 10 | 0.0061490 | adrenomedullin (ADM), mRNA |
| NM_001280.1 | 0 | 9 | 0.0117400 | cold inducible RNA binding protein (CIRBP), mRNA |
| NM_001616.2 | 0 | 13 | 0.0006470 | activin A receptor, type II (ACVR2) |
| NM_001663.2 | 2 | 16 | 0.0026547 | ADP-ribosylation factor 6 (ARF6), mRNA |
| NM_001697.1 | 1 | 12 | 0.0111660 | ATP synthase, H+ transporting, mitochondrial F1 |
| | | | | complex, O subunit (oligomycin sensitivityconferring |
| | | | | protein) (ATP5O) |
| NM_001826.1 | 2 | 9 | 0.0005954 | DC28 protein kinase 1, clone MGC: 12835 |
| | | | | IMAGE: 4110344, mRNA, complete cds. |
| NM_001894.2 | 0 | 9 | 0.0117400 | casein kinase 1, epsilon (CSNK1E) |
| NM_001894.2 | 0 | 13 | 0.0006473 | casein kinase 1, epsilon (CSNK1E) |
| NM_001896.1 | 0 | 10 | 0.0061490 | casein kinase 2, alpha prime polypeptide |
| | | | | (CSNK2A2) |
| NM_001896.2 | 0 | 10 | 0.0061490 | casein kinase 2, alpha prime polypeptide |
| | | | | (CSNK2A2), mRNA |
| NM_002019.1 | 10 | 11 | 0.0117396 | fms-related tyrosine kinase 1 (vascular endothelial |
| | | | | growth factor/vascular permeability factorreceptor) |
| | | | | (FLT1) |
| NM_002103.3 | 0 | 10 | 0.0061493 | glycogen synthase 1 (muscle) (GYS1), mRNA |
| NM_002129.2 | 0 | 10 | 0.0061490 | high-mobility group box 2 (HMGB2), mRNA |
| NM_002387.1 | 10 | 11 | 0.0117400 | mutated in colorectal cancers (MCC), mRNA |
| NM_002462.2 | 1 | 12 | 0.0111658 | myxovirus (influenza virus) resistance 1, interferon- |
| | | | | inducible protein p78 (mouse) (MX1), mRN |
| NM_003045.3 | 1 | 12 | 0.0111658 | solute carrier family 7 (cationic amino acid |
| | | | | transporter, y+ system), member 1 (SLC7A1), mRNA |
| NM_003049.1 | 0 | 9 | 0.0117396 | solute carrier family 10 (sodium/bile acid |
| | | | | cotransporter family), member 1 (SLC10A1), mRNA |
| NM_003295.1 | 0 | 10 | 0.0061490 | tumor protein, translationally-controlled 1 (TPT1), |
| | | | | mRNA |
| NM_003495.2 | 0 | 9 | 0.0117400 | histone 1, H4i (HIST1H4I), mRNA |
| NM_003516.2 | 6 | 20 | 0.0076630 | histone 2, H2aa (HIST2H2AA), mRNA |
| NM_003583.2 | 2 | 15 | 0.0062400 | dual-specificity tyrosine-(Y)-phosphorylation |
| | | | | regulated kinase 2 (DYRK2), transcript variant 1 |
| | | | | mRNA |
| NM_003600.1 | 0 | 9 | 0.0117400 | Serine/threonine kinase 6 (STK6) |
| NM_003662.1 | 2 | 7 | 0.0185217 | Pirin (PIR) |
| NM_003897.2 | 0 | 11 | 0.0030750 | immediate early response 3 (IER3), transcript |
| | | | | variant short, mRNA |
| NM_003907.1 | 3 | 8 | 0.0148845 | Eukaryotic translation initiation factor 2B, subunit 5 |
| | | | | epsilon, 82 kDa (EIF2B5), mRNA |
| NM_004055.3 | 3 | 17 | 0.0048444 | calpain 5 (CAPN5), mRNA |
| NM_004055.3 | 3 | 8 | 0.0148845 | calpain 5 (CAPN5), mRNA |
| NM_004214.3 | 1 | 15 | 0.0010990 | fibroblast growth factor (acidic) intracellular binding |
| | | | | protein (FIBP) |
| NM_004217.1 | 0 | 10 | 0.0061493 | aurora kinase B (AURKB) |
| NM_004567.2 | 10 | 11 | 0.0117400 | 6-phosphofructo-2-kinase/fructose-2,6- |
| | | | | biphosphatase 4 (PFKFB4), mRNA |
| NM_004596.1 | 0 | 13 | 0.0006470 | small nuclear ribonucleoprotein polypeptide A |
| | | | | (SNRPA |
| NM_004635.2 | 1 | 6 | 0.0173851 | mitogen-activated protein kinase-activated protein |
| | | | | kinase 3 (MAPKAPK3) |
| NM_004645.1 | 4 | 17 | 0.0183900 | coilin (COIL), |
| NM_004656.2 | 2 | 15 | 0.0062400 | BRCA1 associated protein-1 (ubiquitin carboxy- |
| | | | | terminal hydrolase) (BAP1), mRNA |
| NM_004732.1 | 4 | 17 | 0.0183901 | potassium voltage-gated channel, shaker-related |
| | | | | subfamily, beta member 3 (KCNAB3) |
| NM_004765.2 | 0 | 10 | 0.0061490 | B-cell CLL/lymphoma 7C (BCL7C), mRNA |
| NM_005157.2 | 1 | 6 | 0.0173851 | v-abl Abelson murine leukemia viral oncogene |
| | | | | homolog 1 (ABL1), transcript variant a |
| NM_005240.1 | 1 | 12 | 0.0111660 | ets variant gene 3 (ETV3), mRNA |
| NM_005435.2 | 2 | 7 | 0.0185217 | Rho guanine nucleotide exchange factor (GEF) 5 |
| | | | | (ARHGEF5) |
| NM_005522.3 | 0 | 11 | 0.0030747 | homeo box A1 (HOXA1), transcript variant 1, mRNA |
| NM_006205.1 | 3 | 18 | 0.0016570 | phosphodiesterase 6H, cGMP-specific, cone, |
| | | | | gamma (PDE6H), mRNA |
| NM_006205.1 | 0 | 6 | 0.0030960 | phosphodiesterase 6H, cGMP-specific, cone, |
| | | | | gamma (PDE6H), mRNA |
| NM_006223.1 | 3 | 16 | 0.0116170 | protein (peptidyl-prolyl cis/trans isomerase) NIMA- |
| | | | | interacting, 4 (parvulin) (PIN4) |
| NM_006298.2 | 1 | 13 | 0.0055970 | zinc finger protein 192 (ZNF192), mRNA |
| NM_006298.2 | 10 | 11 | 0.0117396 | zinc finger protein 192 (ZNF192), mRNA |
| NM_006388.2 | 3 | 17 | 0.0048440 | HIV-1 Tat interacting protein, 60 kDa (HTATIP), |
| | | | | transcript variant 2, mRNA |
| NM_006433.2 | 0 | 9 | 0.0117400 | granulysin (GNLY), transcript variant NKG5, mRNA |
| NM_006607.1 | 4 | 17 | 0.0183900 | pituitary tumor-transforming 2 (PTTG2), mRNA |
| NM_006857.1 | 1 | 12 | 0.0111660 | putative nucleic acid binding protein RY-1 (RY1), |
| | | | | mRNA |
| NM_006869.1 | 0 | 9 | 0.0117396 | centaurin, alpha 1 (CENTA1), mRNA |
| NM_007285.5 | 3 | 16 | 0.0116170 | GABA(A) receptor-associated protein-like 2 |
| | | | | (GABARAPL2) |
| NM_007311.2 | 10 | 11 | 0.0117400 | benzodiazapine receptor (peripheral) (BZRP), |
| | | | | transcript variant PBR-S |
| NM_012163.1 | 0 | 9 | 0.0117400 | F-box and leucine-rich repeat protein 9 (FBXL9) |
| NM_012163.1 | 0 | 10 | 0.0061493 | F-box and leucine-rich repeat protein 9 (FBXL9) |
| NM_012241.2 | 1 | 13 | 0.0055972 | sirtuin (silent mating type information regulation 2 |
| | | | | homolog) 5 (S. cerevisiae) (SIRT5), transcript |
| | | | | variant 1, mRNA |
| NM_012321.1 | 2 | 14 | 0.0130930 | U6 snRNA-associated Sm-like protein (LSM4) |
| NM_013322.2 | 3 | 8 | 0.0148845 | sorting nexin 10 (SNX10), mRNA |
| NM_014765.1 | 0 | 5 | 0.0108359 | translocase of outer mitochondrial membrane 20 |
| | | | | homolog (yeast) (TOMM20), mRNA |
| NM_015488.1 | 3 | 16 | 0.0116170 | myofibrillogenesis regulator 1 (MR-1) |
| NM_015640.1 | 4 | 18 | 0.0072350 | PAI-1 mRNA-binding protein (PAI-RBP1) |
| NM_015987.2 | 0 | 13 | 0.0006470 | heme binding protein 1 (HEBP1) |
| NM_016207.2 | 10 | 11 | 0.0117400 | cleavage and polyadenylation specific factor 3, |
| | | | | 73 kDa (CPSF3), mRNA |
| NM_016355.3 | 1 | 14 | 0.0025990 | DEAD (Asp-Glu-Ala-Asp) box polypeptide 47 |
| | | | | (DDX47), transcript variant 1, mRNA |
| NM_016483.3 | 1 | 15 | 0.0010990 | PHD finger protein 7 (PHF7) |
| NM_016505.2 | 4 | 19 | 0.0021220 | putative S1 RNA binding domain protein (PS1D), |
| | | | | mRNA |
| NM_016576.2 | 0 | 10 | 0.0061493 | guanosine monophosphate reductase 2 (GMPR2) |
| NM_016836.1 | 0 | 5 | 0.0108359 | RNA binding motif, single stranded interacting |
| | | | | protein 1 (RBMS1), transcript variant YC1 |
| NM_016940.1 | 1 | 12 | 0.0111660 | chromosome 21 open reading frame 6 (C21orf6), |
| | | | | mRNA |
| NM_018032.2 | 1 | 13 | 0.0055970 | LUC7-like (S. cerevisiae) (LUC7L) |
| NM_018047.1 | 0 | 9 | 0.0117400 | RNA binding motif protein 22 (RBM22), mRNA |
| NM_018047.1 | 0 | 9 | 0.0117396 | RNA binding motif protein 22 (RBM22), mRNA |
| NM_018107.2 | 1 | 14 | 0.0025990 | RNA-binding region (RNP1, RRM) containing 4 |
| | | | | (RNPC4) |
| NM_018153.2 | 2 | 14 | 0.0130935 | anthrax toxin receptor 1 (ANTXR1), transcript |
| | | | | variant 3, mRNA |
| NM_018184.1 | 0 | 10 | 0.0061493 | ADP-ribosylation factor-like 10C (ARL10C) |
| NM_018679.2 | 0 | 10 | 0.0061493 | t-complex 11 (mouse) (TCP11), mRNA |
| NM_019021.1 | 0 | 12 | 0.0014564 | hypothetical protein FLJ20010 (FLJ20010), mRNA |
| NM_020239.2 | 1 | 12 | 0.0111660 | small protein effector 1 of Cdc42 |
| NM_020317.2 | NA | NA | NA | hypothetical protein dJ465N24.2.1 |
| NM_020367.2 | 0 | 9 | 0.0117396 | chromosome 12 open reading frame 6 (C12orf6) |
| NM_020381.2 | 1 | 14 | 0.0025987 | chromosome 6 open reading frame 210 (C6orf210), |
| | | | | mRNA |
| NM_020444.2 | 10 | 11 | 0.0117400 | KIAA1191 protein (KIAA1191), mRNA |
| NM_020661.1 | 1 | 13 | 0.0055970 | activation-induced cytidine deaminase (AICDA), |
| | | | | mRNA |
| NM_020804.2 | 0 | 9 | 0.0117396 | protein kinase C and casein kinase substrate in |
| | | | | neurons 1 (PACSIN1), mRNA |
| NM_020898.1 | 2 | 7 | 0.0185217 | KIAA1536 protein (KIAA1536), mRNA |
| NM_021104.1 | 1 | 13 | 0.0055970 | ribosomal protein L41 (RPL41), mRNA |
| NM_021130.1 | 10 | 11 | 0.0117396 | peptidylprolyl isomerase A (cyclophilin A) (PPIA) |
| NM_021133.1 | 3 | 16 | 0.0116173 | ribonuclease L (2′,5′-oligoisoadenylate synthetase- |
| | | | | dependent) (RNASEL), |
| NM_021639.2 | 0 | 5 | 0.0108359 | hypothetical protein SP192 (SP192) |
| NM_021709.1 | 0 | 10 | 0.0061493 | CD27-binding (Siva) protein (SIVA), transcript |
| | | | | variant 2, mRNA |
| NM_021822.1 | 0 | 9 | 0.0117400 | apolipoprotein B mRNA editing enzyme, catalytic |
| | | | | polypeptide-like 3G (APOBEC3G), mRNA |
| NM_022100.1 | 0 | 9 | 0.0117400 | mitochondrial ribosomal protein S14 (MRPS14), |
| | | | | nuclear gene encoding mitochondrial protein, mRNA |
| NM_022787.2 | 4 | 19 | 0.0021220 | nicotinamide nucleotide adenylyltransferase 1 |
| | | | | (NMNAT1), mRNA |
| NM_022787.2 | 4 | 17 | 0.0183901 | nicotinamide nucleotide adenylyltransferase 1 |
| | | | | (NMNAT1), mRNA |
| NM_023940.1 | 10 | 11 | 0.0117400 | hypothetical protein MGC2827 |
| NM_024292.2 | 0 | 5 | 0.0108359 | ubiquitin-like 5, mRNA, complete cds. |
| NM_024625.3 | 0 | 9 | 0.0117400 | zinc finger CCCH type, antiviral 1 (ZC3HAV1), |
| | | | | transcript variant 2, mRNA |
| NM_031465.2 | 0 | 11 | 0.0030750 | hypothetical protein, mRNA |
| NM_031473.1 | 2 | 7 | 0.0185217 | carnitine deficiency-associated gene expressed in |
| | | | | ventricle 1 (CDV-1) |
| NM_032042.2 | 0 | 12 | 0.0014560 | hypothetical protein DKFZp564D172 |
| | | | | (DKFZP564D172) |
| NM_032042.2 | 1 | 6 | 0.0173851 | hypothetical protein DKFZp564D172 |
| NM_032328.1 | 0 | 5 | 0.0108359 | hypothetical protein, mRNA |
| NM_032345.1 | 0 | 10 | 0.0061490 | PYM protein (PYM), mRNA |
| NM_032350.3 | 0 | 9 | 0.0117400 | hypothetical protein, mRNA |
| NM_032855.1 | 0 | 12 | 0.0014564 | hematopoietic SH2 protein (HSH2) |
| NM_032855.1 | 1 | 6 | 0.0173851 | hematopoietic SH2 protein (HSH2) |
| NM_032906.2 | 2 | 15 | 0.0062400 | hypothetical protein, mRNA |
| NM_033030.2 | 1 | 12 | 0.0111660 | bol, boule-like (Drosophila) (BOLL) |
| NM_033122.1 | 3 | 8 | 0.0148845 | testis development protein NYD-SP26 (NYD-SP26), |
| NM_052822.1 | 0 | 9 | 0.0117396 | secretory carrier membrane protein 1 (SCAMP1), |
| | | | | transcript variant 2 |
| NM_052877.1 | 0 | 12 | 0.0014564 | mediator of RNA polymerase II transcription, subunit |
| | | | | 8 homolog (yeast) (MED8) |
| NM_054016.1 | 1 | 14 | 0.0025990 | FUS interacting protein (serine-arginine rich) 1 |
| | | | | (FUSIP1), transcript variant 2, mRNA |
| NM_138551.1 | 3 | 16 | 0.0116170 | thymic stromal lymphopoietin (TSLP), transcript |
| | | | | variant 2 |
| NM_138775.1 | 1 | 12 | 0.0111660 | hypothetical protein BC015183 (LOC91801), mRNA |
| NM_144982.1 | 2 | 15 | 0.0062400 | hypothetical protein MGC23401 (MGC23401) |
| NM_145020.1 | NA | NA | NA | hypothetical protein FLJ32743 |
| NM_145315.2 | 10 | 11 | 0.0117396 | lactation elevated 1 (LACE1) |
| NM_145810.1 | 0 | 9 | 0.0117400 | cell division cycle associated 7 (CDCA7), transcript |
| | | | | variant 2, mRNA |
| NM_152688.1 | 0 | 11 | 0.0030750 | KH domain containing, RNA binding, signal |
| | | | | transduction associated 2 (KHDRBS2), mRNA |
| NM_152688.1 | 0 | 10 | 0.0061493 | KH domain containing, RNA binding, signal |
| | | | | transduction associated 2 (KHDRBS2), mRNA |
| NM_152697.2 | 3 | 16 | 0.0116173 | hypothetical protein, mRNA |
| NM_152769.1 | 0 | 11 | 0.0030750 | chromosome 19 open reading frame 26 (C19orf26), |
| | | | | mRNA |
| NM_152770.1 | 1 | 12 | 0.0111660 | hypothetical protein, mRNA |
| NM_152770.1 | 0 | 5 | 0.0108359 | hypothetical protein, mRNA |
| NM_153207.2 | 2 | 15 | 0.0062400 | AE binding protein 2 (AEBP2) |
| NM_153215.1 | 1 | 17 | 0.0001340 | hypothetical protein FLJ38608 (FLJ38608), mRNA |
| NM_153332.2 | 4 | 19 | 0.0021220 | 3′ exoribonuclease (3′HEXO), mRNA |
| NM_173474.2 | 0 | 5 | 0.0108359 | N-terminal asparagine amidase (NTAN1), mRNA |
| NM_173519.1 | 0 | 5 | 0.0108359 | hypothetical protein, mRNA |
| NM_173545.1 | 0 | 9 | 0.0117400 | chromosome 2 open reading frame 13 (C2orf13), |
| | | | | mRNA |
| NM_175923.2 | 1 | 12 | 0.0111660 | hypothetical protein MGC42630 (MGC42630) |
| NM_177996.1 | 1 | 12 | 0.0111660 | erythrocyte membrane protein band 4.1-like 1 |
| | | | | (EPB41L1), transcript variant 2, mRNA |
| NM_177996.1 | 1 | 6 | 0.0173851 | erythrocyte membrane protein band 4.1-like 1 |
| | | | | (EPB41L1), transcript variant 2, mRNA |
| NM_178496.2 | 1 | 13 | 0.0055972 | similar to BcDNA:GH11415 gene product |
| | | | | (LOC151963), mRNA |
| NM_182623.1 | 4 | 18 | 0.0072350 | hypothetical protein FLJ36766 (FLJ36766), mRNA |
| NM_182665.1 | 0 | 12 | 0.0014564 | Ras association (RalGDS/AF-6) domain family 5 |
| | | | | (RASSF5), transcript variant 3, mRNA |
| NM_198395.1 | 0 | 9 | 0.0117400 | Ras-GTPase-activating protein SH3-domain-binding |
| | | | | protein (G3BP), transcript variant 2 |
| NM_198490.1 | 0 | 11 | 0.0030747 | RAB43, member RAS oncogene family (RAB43), |
| | | | | mRNA |
| NM_203326.1 | 0 | 9 | 0.0117396 | 5-azacytidine induced 2 (AZI2), transcript variant 2 |
| NM_203326.1 | 0 | 5 | 0.0108359 | 5-azacytidine induced 2 (AZI2), transcript variant 2 |
| NM_212492.1 | 0 | 10 | 0.0061493 | G protein pathway suppressor 1 (GPS1), transcript |
| | | | | variant 1, mRNA |

Table 2 is a list of autoantigens that were bound by antibodies in sera from individuals with RA (before treatment with infliximab) more often than by antibodies in sera from healthy individuals. The normal count and RA count are presented along with the corresponding p-value.

TABLE 2
RA vs. healthy patients

| Genbank ID | | | | |
|---|---|---|---|---|
| number of | | | | |
| nucleic acid | | | | |
| coding for | Normal | RA | | |
| the protein | Count | Count | p-value | Name or description |
| BC000809.1 | 2 | 7 | 0.018522 | transcription elongation factor A (SII)-like 1 |
| BC001662.1 | 1 | 6 | 0.017385 | mitogen-activated protein kinase-activated protein |
| | | | | kinase 3 |
| BC002637.1 | 0 | 5 | 0.010836 | tribbles homolog 2 |
| BC004514.1 | 1 | 7 | 0.004882 | hypothetical protein FLJ12584 |
| BC007411.2 | 2 | 7 | 0.018522 | diaphanous homolog 1 (Drosophila) |
| BC007863.1 | 0 | 5 | 0.010836 | platelet-activating factor acetylhydrolase, isoform lb, |
| | | | | gamma subunit (29 kD) |
| BC011863.2 | 2 | 7 | 0.018522 | Unknown (protein for MGC: 20604) |
| BC012105.1 | 3 | 8 | 0.014884 | nuclear VCP-like, mRNA |
| BC013103.1 | 1 | 6 | 0.017385 | Similar to hypothetical protein FLJ20435, |
| | | | | cloneMGC: 16997 IMAGE: 4343882, mRNA, |
| | | | | complete cds. |
| BC014435.1 | 0 | 5 | 0.010836 | Unknown (protein for MGC: 22922) |
| BC014975.1 | 2 | 7 | 0.018522 | hypothetical protein FLJ14668, mRNA |
| BC016381.1 | 1 | 6 | 0.017385 | cDNA clone MGC: 27378 IMAGE: 4688865, complete |
| | | | | cds |
| BC018142.1 | 3 | 8 | 0.014884 | caspase recruitment domain family, member 14, |
| | | | | mRNA |
| BC018302.1 | 0 | 6 | 0.003096 | TRM1 tRNA methyltransferase 1 homolog (S. cerevisiae), |
| | | | | mRNA |
| BC024289.1 | 1 | 7 | 0.004882 | cDNA clone MGC: 39273 IMAGE: 5440834, complete |
| | | | | cds |
| BC025314.1 | 1 | 6 | 0.017385 | immunoglobulin heavy constant gamma 1 (G1m |
| | | | | marker), mRNA |
| BC028039.1 | 1 | 6 | 0.017385 | hypothetical protein MGC39900 |
| BC030219.1 | 1 | 6 | 0.017385 | RAD51-like 1 (S. cerevisiae) |
| BC032124.1 | 1 | 6 | 0.017385 | bromodomain containing 3 |
| BC032334.1 | 0 | 5 | 0.010836 | putative homeodomain transcription factor 2, mRNA, |
| | | | | complete cds. |
| BC033195.1 | 1 | 6 | 0.017385 | leukocyte receptor cluster (LRC) member 12 |
| BC034954.2 | 2 | 7 | 0.018522 | nucleosome assembly protein 1-like 3, mRNA |
| BC037906.1 | 2 | 7 | 0.018522 | hypothetical protein FLJ11017, mRNA |
| BC041037.1 | 0 | 7 | 0.000714 | immunoglobulin heavy constant mu, mRNA |
| CTL2132 | 1 | 6 | 0.017385 | myeloperoxidase_100 ug/ml_S |
| NM_001826.1 | 2 | 9 | 0.000595 | CDC28 protein kinase 1, clone MGC: 12835 |
| | | | | IMAGE: 4110344, mRNA, complete cds. |
| NM_003662.1 | 2 | 7 | 0.018522 | Pirin (PIR) |
| NM_003907.1 | 3 | 8 | 0.014884 | eukaryotic translation initiation factor 2B, subunit 5 |
| | | | | epsilon, 82 kDa (EIF2B5), mRNA |
| NM_004055.3 | 3 | 8 | 0.014884 | calpain 5 (CAPN5), mRNA |
| NM_004635.2 | 1 | 6 | 0.017385 | mitogen-activated protein kinase-activated protein |
| | | | | kinase 3 (MAPKAPK3) |
| NM_005157.2 | 1 | 6 | 0.017385 | v-abl Abelson murine leukemia viral oncogene |
| | | | | homolog 1 (ABL1), transcript variant a |
| NM_005435.2 | 2 | 7 | 0.018522 | Rho guanine nucleotide exchange factor (GEF) 5 |
| | | | | (ARHGEF5) |
| NM_006205.1 | 0 | 6 | 0.003096 | phosphodiesterase 6H, cGMP-specific, cone, |
| | | | | gamma (PDE6H), mRNA |
| NM_013322.2 | 3 | 8 | 0.014884 | sorting nexin 10 (SNX10), mRNA |
| NM_014765.1 | 0 | 5 | 0.010836 | translocase of outer mitochondrial membrane 20 |
| | | | | homolog (yeast) (TOMM20), mRNA |
| NM_016836.1 | 0 | 5 | 0.010836 | RNA binding motif, single stranded interacting |
| | | | | protein 1 (RBMS1), transcript variant YC1 |
| NM_020898.1 | 2 | 7 | 0.018522 | KIAA1536 protein (KIAA1536), mRNA |
| NM_021639.2 | 0 | 5 | 0.010836 | hypothetical protein SP192 (SP192) |
| NM_024292.2 | 0 | 5 | 0.010836 | ubiquitin-like 5, mRNA, complete cds. |
| NM_031473.1 | 2 | 7 | 0.018522 | carnitine deficiency-associated gene expressed in |
| | | | | ventricle 1 (CDV-1) |
| NM_032042.2 | 1 | 6 | 0.017385 | hypothetical protein DKFZp564D172 |
| NM_032328.1 | 0 | 5 | 0.010836 | hypothetical protein, mRNA |
| NM_032855.1 | 1 | 6 | 0.017385 | hematopoietic SH2 protein (HSH2) |
| NM_033122.1 | 3 | 8 | 0.014884 | testis development protein NYD-SP26 (NYD-SP26), |
| NM_152770.1 | 0 | 5 | 0.010836 | hypothetical protein, mRNA |
| NM_173474.2 | 0 | 5 | 0.010836 | N-terminal asparagine amidase (NTAN1), mRNA |
| NM_173519.1 | 0 | 5 | 0.010836 | hypothetical protein, mRNA |
| NM_177996.1 | 1 | 6 | 0.017385 | erythrocyte membrane protein band 4.1-like 1 |
| | | | | (EPB41L1), transcript variant 2, mRNA |
| NM_203326.1 | 0 | 5 | 0.010836 | 5-azacytidine induced 2 (AZI2), transcript variant 2 |

Table 3 is a list of autoantigens that were bound more often by antibodies in sera from individuals with SLE than by antibodies in sera from healthy individuals. The normal count and SLE count are presented along with the corresponding p-value.

TABLE 3
SLE vs. healthy patients

| Genbank ID | | | | |
|---|---|---|---|---|
| number of | | | | |
| nucleic acid | | | | |
| coding for | Normal | SLE | | |
| the protein | Count | Count | p-value | Name or description |
| BC000175.2 | 1 | 12 | 0.011166 | Hermansky-Pudlak syndrome 1, transcript variant 3 |
| BC000381.2 | 1 | 13 | 0.005597 | TBP-like 1, mRNA |
| BC000914.1 | 1 | 15 | 0.001099 | splicing factor, arginine/serine-rich 3 |
| BC000997.2 | 1 | 12 | 0.011166 | splicing factor, arginine/serine-rich 7, 35 kDa |
| BC001396.1 | 3 | 17 | 0.004844 | AD-003 protein |
| BC002733.2 | 1 | 12 | 0.011166 | mRNA, complete cds. |
| BC005248.1 | 0 | 11 | 0.003075 | eukaryotic translation initiation factor 1A, Y-linked |
| BC006376.1 | 0 | 10 | 0.006149 | N-myristoyltransferase 2 |
| BC006456.1 | 10 | 11 | 0.01174 | KIAA0592 protein |
| BC006793.1 | 0 | 10 | 0.006149 | GATA binding protein 3 |
| BC007228.1 | 0 | 9 | 0.01174 | Taxol resistant associated protein 3 (TRAG-3) |
| BC007833.2 | 0 | 9 | 0.01174 | phosphatidylinositol-4-phosphate 5-kinase, type I, |
| | | | | alpha, mRNA |
| BC007888.1 | 2 | 14 | 0.013093 | eukaryotic translation initiation factor 2, subunit 2 |
| | | | | (beta, 38 kD) |
| BC008623.1 | 0 | 12 | 0.001456 | hypothetical protein FLJ21044 similar to Rbig1, |
| | | | | cloneMGC: 16823 IMAGE: 4177689, mRNA, |
| | | | | complete cds. |
| BC009623.1 | 0 | 9 | 0.01174 | nucleophosmin (nucleolar phosphoprotein B23, |
| | | | | numatrin) |
| BC009762.2 | 0 | 9 | 0.01174 | mRNA, complete cds. |
| BC009873.1 | NA | NA | NA | clone MGC: 16442 IMAGE: 3946787 |
| BC010642.1 | 0 | 9 | 0.01174 | zinc finger protein 22 (KOX 15), |
| BC011379.1 | 2 | 15 | 0.00624 | DKFZP434H132 protein |
| BC011498.1 | 4 | 18 | 0.007235 | Unknown (protein for MGC: 17017) |
| BC011668.1 | 1 | 12 | 0.011166 | Similar to casein kinase 2, alpha 1 polypeptide |
| BC011707.1 | 10 | 11 | 0.01174 | nuclear receptor binding factor 2, mRNA |
| BC011804.2 | 4 | 17 | 0.01839 | chromosome 1 open reading frame 165, mRNA |
| BC012120.1 | 0 | 9 | 0.01174 | nuclear factor I/C (CCAAT-binding transcription |
| | | | | factor) |
| BC012472.1 | 1 | 12 | 0.011166 | ubiquitin D, mRNA |
| BC012924.1 | NA | NA | NA | dual adaptor of phosphotyrosine and 3- |
| | | | | phosphoinositides |
| BC013073.1 | 0 | 10 | 0.006149 | chromosome 1 open reading frame 37, mRNA |
| BC013567.1 | 10 | 11 | 0.01174 | hypothetical protein FLJ11328 |
| BC014452.1 | 4 | 17 | 0.01839 | cDNA clone IMAGE: 4903661 |
| BC015008.1 | 4 | 17 | 0.01839 | hydroxyacylglutathione hydrolase-like, mRNA |
| BC015497.1 | 1 | 12 | 0.011166 | cDNA clone MGC: 9014 IMAGE: 3913870, complete |
| | | | | cds |
| BC015715.1 | 1 | 12 | 0.011166 | makorin, ring finger protein, 2 |
| BC016764.1 | 4 | 20 | 0.000354 | ribulose-5-phosphate-3-epimerase, transcript |
| | | | | variant 1 |
| BC016778.1 | 2 | 14 | 0.013093 | HIV-1 rev binding protein 2, mRNA |
| BC016842.1 | 1 | 12 | 0.011166 | family with sequence similarity 61, member A, |
| | | | | mRNA |
| BC017114.1 | 0 | 10 | 0.006149 | hypothetical protein FLJ22833 |
| BC019337.1 | 10 | 11 | 0.01174 | immunoglobulin heavy constant gamma 1 (G1m |
| | | | | marker), mRNA |
| BC020647.1 | 2 | 14 | 0.013093 | HSPC128 protein, mRNA |
| BC022231.1 | 1 | 12 | 0.011166 | Ets2 repressor factor, mRNA |
| BC022325.1 | 0 | 16 | 3.33E−05 | hypothetical protein FLJ12729 |
| BC023569.1 | 0 | 9 | 0.01174 | UPF3 regulator of nonsense transcripts homolog A |
| | | | | (yeast), transcript variant 2 |
| BC025996.2 | 0 | 13 | 0.000647 | cDNA clone MGC: 26787 IMAGE: 4838986 |
| BC027607.1 | 1 | 12 | 0.011166 | clone MGC: 26892 IMAGE: 4828241 |
| BC028151.1 | 0 | 9 | 0.01174 | DNA segment on chromosome X and Y (unique) |
| | | | | 155 expressed sequence, mRNA |
| BC028237.1 | 1 | 12 | 0.011166 | growth differentiation factor 10, mRNA |
| BC028301.1 | 0 | 12 | 0.001456 | mRNA similar to LOC147447 |
| BC029046.1 | 4 | 17 | 0.01839 | H1 histone family, member 0, mRNA |
| BC029827.1 | 0 | 9 | 0.01174 | Down syndrome critical region gene 9, mRNA |
| BC029891.1 | 0 | 10 | 0.006149 | transcription factor EC, mRNA |
| BC030219.1 | 1 | 14 | 0.002599 | RAD51-like 1 (S. cerevisiae) |
| BC030702.1 | 0 | 10 | 0.006149 | hypothetical protein FLJ12847 |
| BC032452.1 | 10 | 11 | 0.01174 | immunoglobulin lambda constant 1 (Mcg marker), |
| | | | | mRNA |
| BC032462.1 | 0 | 10 | 0.006149 | vacuolar protein sorting 29 (yeast), mRNA |
| BC032485.1 | 0 | 9 | 0.01174 | hypothetical protein FLJ30473 |
| BC032852.2 | 4 | 18 | 0.007235 | melanoma antigen family B, 4, mRNA |
| BC033856.1 | 3 | 17 | 0.004844 | Similar to RIKEN cDNA 3110040D16 gene, |
| | | | | cloneMGC: 45395 IMAGE: 5123380, mRNA, |
| | | | | complete cds. |
| BC034401.1 | 10 | 11 | 0.01174 | Similar to LOC161981 |
| BC035314.1 | 4 | 17 | 0.01839 | brix domain containing 1 |
| BC035568.1 | 0 | 9 | 0.01174 | acylphosphatase 1, erythrocyte (common) type |
| BC038105.2 | 1 | 14 | 0.002599 | membrane protein, palmitoylated 7 (MAGUK p55 |
| | | | | subfamily member 7) |
| BC040844.1 | 1 | 14 | 0.002599 | synaptotagmin binding, cytoplasmic RNA |
| | | | | interacting protein, mRNA |
| BC042625.1 | 1 | 13 | 0.005597 | LUC7-like 2 (S. cerevisiae), mRNA |
| BC050428.1 | 5 | 19 | 0.008842 | katanin p60 (ATPase-containing) subunit A 1, |
| | | | | mRNA |
| BC051301.1 | 0 | 10 | 0.006149 | TEA domain family member 2, mRNA |
| BC052806.1 | 0 | 9 | 0.01174 | cDNA clone MGC: 61802 IMAGE: 5730155 |
| BC053656.1 | 10 | 11 | 0.01174 | EGF-like repeats and discoidin I-like domains 3, |
| | | | | mRNA |
| BC053866.1 | 0 | 9 | 0.01174 | endothelin 3, transcript variant 2 |
| BC053872.1 | 0 | 9 | 0.01174 | copine V, mRNA |
| BC054034.1 | 1 | 12 | 0.011166 | U11/U12 snRNP 35K, transcript variant 2 |
| BC055314.1 | 0 | 10 | 0.006149 | C2f protein |
| BC057774.1 | 1 | 12 | 0.011166 | hypothetical protein FLJ31455, mRNA |
| BC058903.1 | 0 | 9 | 0.01174 | intercellular adhesion molecule 3, mRNA |
| BC063275.1 | 1 | 15 | 0.001099 | eukaryotic translation initiation factor 2C, 1, mRNA |
| BC063479.1 | 1 | 15 | 0.001099 | La ribonucleoprotein domain family, member 4, |
| | | | | mRNA |
| BC067446.1 | 0 | 9 | 0.01174 | disabled homolog 1 (Drosophila), mRNA |
| CTL1094 | 10 | 11 | 0.01174 | Influenza A |
| CTL2110 | 0 | 11 | 0.003075 | DNA TOPOISIMERASE(Scl-70) |
| CTL2112 | 3 | 17 | 0.004844 | ssDNA |
| CTL2132 | 1 | 12 | 0.011166 | myeloperoxidase |
| CTL2136 | 1 | 13 | 0.005597 | U1-snRNP 68 PROTEIN |
| CTL2138 | 0 | 14 | 0.000267 | RNP COMPLEX |
| CTL2142 | 4 | 19 | 0.002122 | ssDNA |
| CTL2145 | 0 | 14 | 0.000267 | RIBOSOMAL RNA |
| CTL2152 | 2 | 14 | 0.013093 | RNA POLYMERASE |
| NM_000997.2 | 0 | 9 | 0.01174 | ribosomal protein L37 (RPL37) |
| NM_001014.2 | 1 | 14 | 0.002599 | ribosomal protein S10 (RPS10) |
| NM_001106.2 | 1 | 12 | 0.011166 | activin A receptor, type IIB (ACVR2B) |
| NM_001124.1 | 0 | 10 | 0.006149 | adrenomedullin (ADM), mRNA |
| NM_001280.1 | 0 | 9 | 0.01174 | cold inducible RNA binding protein (CIRBP), mRNA |
| NM_001616.2 | 0 | 13 | 0.000647 | activin A receptor, type II (ACVR2) |
| NM_001697.1 | 1 | 12 | 0.011166 | ATP synthase, H+ transporting, mitochondrial F1 |
| | | | | complex, O subunit (oligomycin sensitivity |
| | | | | conferring protein) (ATP5O) |
| NM_001894.2 | 0 | 9 | 0.01174 | casein kinase 1, epsilon (CSNK1E) |
| NM_001896.1 | 0 | 10 | 0.006149 | casein kinase 2, alpha prime polypeptide |
| | | | | (CSNK2A2) |
| NM_001896.2 | 0 | 10 | 0.006149 | casein kinase 2, alpha prime polypeptide |
| | | | | (CSNK2A2), mRNA |
| NM_002129.2 | 0 | 10 | 0.006149 | high-mobility group box 2 (HMGB2), mRNA |
| NM_002387.1 | 10 | 11 | 0.01174 | mutated in colorectal cancers (MCC), mRNA |
| NM_003295.1 | 0 | 10 | 0.006149 | tumor protein, translationally-controlled 1 (TPT1), |
| | | | | mRNA |
| NM_003495.2 | 0 | 9 | 0.01174 | histone 1, H4i (HIST1H4I), mRNA |
| NM_003516.2 | 6 | 20 | 0.007663 | histone 2, H2aa (HIST2H2AA), mRNA |
| NM_003583.2 | 2 | 15 | 0.00624 | dual-specificity tyrosine-(Y)-phosphorylation |
| | | | | regulated kinase 2 (DYRK2), transcript variant 1, |
| | | | | mRNA |
| NM_003600.1 | 0 | 9 | 0.01174 | serine/threonine kinase 6 (STK6) |
| NM_003897.2 | 0 | 11 | 0.003075 | immediate early response 3 (IER3), transcript |
| | | | | variant short, mRNA |
| NM_004214.3 | 1 | 15 | 0.001099 | fibroblast growth factor (acidic) intracellular binding |
| | | | | protein (FIBP) |
| NM_004567.2 | 10 | 11 | 0.01174 | 6-phosphofructo-2-kinase/fructose-2 6- |
| | | | | biphosphatase 4 (PFKFB4), mRNA |
| NM_004596.1 | 0 | 13 | 0.000647 | small nuclear ribonucleoprotein polypeptide A |
| | | | | (SNRPA |
| NM_004645.1 | 4 | 17 | 0.01839 | coilin (COIL), |
| NM_004656.2 | 2 | 15 | 0.00624 | BRCA1 associated protein-1 (ubiqutin carboxy- |
| | | | | terminal hydrolase) (BAP1), mRNA |
| NM_004765.2 | 0 | 10 | 0.006149 | B-cell CLL/lymphoma 7C (BCL7C), mRNA |
| NM_005240.1 | 1 | 12 | 0.011166 | ets variant gene 3 (ETV3), mRNA |
| NM_006205.1 | 3 | 18 | 0.001657 | phosphodiesterase 6H, cGMP-specific, cone, |
| | | | | gamma (PDE6H), mRNA |
| NM_006223.1 | 3 | 16 | 0.011617 | protein (peptidyl-prolyl cis/trans isomerase) NIMA- |
| | | | | interacting, 4 (parvulin) (PIN4) |
| NM_006298.2 | 1 | 13 | 0.005597 | zinc finger protein 192 (ZNF192), mRNA |
| NM_006388.2 | 3 | 17 | 0.004844 | HIV-1 Tat interacting protein, 60 kDa (HTATIP), |
| | | | | transcript variant 2, mRNA |
| NM_006433.2 | 0 | 9 | 0.01174 | granulysin (GNLY), transcript variant NKG5, mRNA |
| NM_006607.1 | 4 | 17 | 0.01839 | pituitary tumor-transforming 2 (PTTG2), mRNA |
| NM_006857.1 | 1 | 12 | 0.011166 | putative nucleic acid binding protein RY-1 (RY1), |
| | | | | mRNA |
| NM_007285.5 | 3 | 16 | 0.011617 | GABA(A) receptor-associated protein-like 2 |
| | | | | (GABARAPL2) |
| NM_007311.2 | 10 | 11 | 0.01174 | benzodiazapine receptor (peripheral) (BZRP), |
| | | | | transcript variant PBR-S |
| NM_012163.1 | 0 | 9 | 0.01174 | F-box and leucine-rich repeat protein 9 (FBXL9) |
| NM_012321.1 | 2 | 14 | 0.013093 | U6 snRNA-associated Sm-like protein (LSM4) |
| NM_015488.1 | 3 | 16 | 0.011617 | myofibrillogenesis regulator 1 (MR-1) |
| NM_015640.1 | 4 | 18 | 0.007235 | PAI-1 mRNA-binding protein (PAI-RBP1) |
| NM_015987.2 | 0 | 13 | 0.000647 | heme binding protein 1 (HEBP1) |
| NM_016207.2 | 10 | 11 | 0.01174 | cleavage and polyadenylation specific factor 3, |
| | | | | 73 kDa (CPSF3), mRNA |
| NM_016355.3 | 1 | 14 | 0.002599 | DEAD (Asp-Glu-Ala-Asp) box polypeptide 47 |
| | | | | (DDX47), transcript variant 1, mRNA |
| NM_016483.3 | 1 | 15 | 0.001099 | PHD finger protein 7 (PHF7) |
| NM_016505.2 | 4 | 19 | 0.002122 | putative S1 RNA binding domain protein (PS1D), |
| | | | | mRNA |
| NM_016940.1 | 1 | 12 | 0.011166 | chromosome 21 open reading frame 6 (C21orf6), |
| | | | | mRNA |
| NM_018032.2 | 1 | 13 | 0.005597 | LUC7-like (S. cerevisiae) (LUC7L) |
| NM_018047.1 | 0 | 9 | 0.01174 | RNA binding motif protein 22 (RBM22), mRNA |
| NM_018107.2 | 1 | 14 | 0.002599 | RNA-binding region (RNP1, RRM) containing 4 |
| | | | | (RNPC4) |
| NM_020239.2 | 1 | 12 | 0.011166 | small protein effector 1 of Cdc42 |
| NM_020317.2 | NA | NA | NA | hypothetical protein dJ465N24.2.1 |
| NM_020444.2 | 10 | 11 | 0.01174 | KIAA1191 protein (KIAA1191), mRNA |
| NM_020661.1 | 1 | 13 | 0.005597 | activation-induced cytidine deaminase (AICDA), |
| | | | | mRNA |
| NM_021104.1 | 1 | 13 | 0.005597 | ribosomal protein L41 (RPL41), mRNA |
| NM_021822.1 | 0 | 9 | 0.01174 | apolipoprotein B mRNA editing enzyme, catalytic |
| | | | | polypeptide-like 3G (APOBEC3G), mRNA |
| NM_022100.1 | 0 | 9 | 0.01174 | mitochondrial ribosomal protein S14 (MRPS14), |
| | | | | nuclear gene encoding mitochondrial protein, |
| | | | | mRNA |
| NM_022787.2 | 4 | 19 | 0.002122 | nicotinamide nucleotide adenylyltransferase 1 |
| | | | | (NMNAT1), mRNA |
| NM_023940.1 | 10 | 11 | 0.01174 | hypothetical protein MGC2827 |
| NM_024625.3 | 0 | 9 | 0.01174 | zinc finger CCCH type, antiviral 1 (ZC3HAV1), |
| | | | | transcript variant 2, mRNA |
| NM_031465.2 | 0 | 11 | 0.003075 | hypothetical protein, mRNA |
| NM_032042.2 | 0 | 12 | 0.001456 | hypothetical protein DKFZp564D172 |
| | | | | (DKFZP564D172) |
| NM_032345.1 | 0 | 10 | 0.006149 | PYM protein (PYM), mRNA |
| NM_032350.3 | 0 | 9 | 0.01174 | hypothetical protein, mRNA |
| NM_032906.2 | 2 | 15 | 0.00624 | hypothetical protein, mRNA |
| NM_033030.2 | 1 | 12 | 0.011166 | bol, boule-like (Drosophila) (BOLL) |
| NM_054016.1 | 1 | 14 | 0.002599 | FUS interacting protein (serine-arginine rich) 1 |
| | | | | (FUSIP1), transcript variant 2, mRNA |
| NM_138551.1 | 3 | 16 | 0.011617 | thymic stromal lymphopoietin (TSLP), transcript |
| | | | | variant 2 |
| NM_138775.1 | 1 | 12 | 0.011166 | hypothetical protein BC015183 (LOC91801), |
| | | | | mRNA |
| NM_144982.1 | 2 | 15 | 0.00624 | hypothetical protein MGC23401 (MGC23401) |
| NM_145020.1 | NA | NA | NA | hypothetical protein FLJ32743 |
| NM_145810.1 | 0 | 9 | 0.01174 | cell division cycle associated 7 (CDCA7), transcript |
| | | | | variant 2, mRNA |
| NM_152688.1 | 0 | 11 | 0.003075 | KH domain containing, RNA binding, signal |
| | | | | transduction associated 2 (KHDRBS2), mRNA |
| NM_152769.1 | 0 | 11 | 0.003075 | chromosome 19 open reading frame 26 |
| | | | | (C19orf26), mRNA |
| NM_152770.1 | 1 | 12 | 0.011166 | hypothetical protein, mRNA |
| NM_153207.2 | 2 | 15 | 0.00624 | AE binding protein 2 (AEBP2) |
| NM_153332.2 | 4 | 19 | 0.002122 | 3′ exoribonuclease (3′HEXO), mRNA |
| NM_173545.1 | 0 | 9 | 0.01174 | chromosome 2 open reading frame 13 (C2orf13), |
| | | | | mRNA |
| NM_175923.2 | 1 | 12 | 0.011166 | hypothetical protein MGC42630 (MGC42630) |
| NM_177996.1 | 1 | 12 | 0.011166 | erythrocyte membrane protein band 4.1-like 1 |
| | | | | (EPB41L1), transcript variant 2, mRNA |
| NM_182623.1 | 4 | 18 | 0.007235 | hypothetical protein FLJ36766 (FLJ36766), mRNA |
| NM_198395.1 | 0 | 9 | 0.01174 | Ras-GTPase-activating protein SH3-domain- |
| | | | | binding protein (G3BP), transcript variant 2 |

The autoantigens listed in Table 4 are selective for SLE, but not RA or ANCA. Table 4 is a list of autoantigens that were bound by an antibody from sera from an individual with SLE but not healthy, RA or ANCA patients.

TABLE 4
SLE vs. all

| Genbank ID | |
|---|---|
| number of nucleic | |
| acid coding for | |
| the protein | Name or Description |
| BC000381.2 | TBP-like 1, mRNA |
| BC002733.2 | mRNA, complete cds. |
| BC006376.1 | N-myristoyltransferase 2 |
| BC007833.2 | phosphatidylinositol-4-phosphate 5-kinase, type I, alpha, mRNA |
| BC008623.1 | hypothetical protein FLJ21044 similar to Rbig1, cloneMGC: 16823 |
| | IMAGE: 4177689, mRNA, complete cds. |
| BC009623.1 | Similar to nucleophosmin (nucleolar phosphoprotein B23, numatrin) |
| BC009762.2 | mRNA, complete cds. |
| BC010642.1 | zinc finger protein 22 (KOX 15) |
| BC011498.1 | histone deacetylase 6 |
| BC012472.1 | ubiquitin D, mRNA |
| BC014452.1 | cDNA clone IMAGE: 4903661 |
| BC015008.1 | hydroxyacylglutathione hydrolase-like, mRNA |
| BC016842.1 | family with sequence similarity 61, member A, mRNA |
| BC017114.1 | hypothetical protein FLJ22833 |
| BC020647.1 | HSPC128 protein, mRNA |
| BC022325.1 | hypothetical protein FLJ12729 |
| BC025996.2 | CDNA clone MGC: 26787 IMAGE: 4838986 |
| BC027607.1 | clone MGC: 26892 IMAGE: 4828241 |
| BC028301.1 | mRNA similar to LOC147447 |
| BC029046.1 | H1 histone family, member 0, mRNA |
| BC032852.2 | melanoma antigen family B, 4, mRNA |
| BC033856.1 | Similar to RIKEN cDNA 3110040D16 gene, cloneMGC: 45395 |
| | IMAGE: 5123380, mRNA, complete cds. |
| BC038105.2 | membrane protein, palmitoylated 7 (MAGUK p55 subfamily member 7) |
| BC042625.1 | LUC7-like 2 (S. cerevisiae), mRNA |
| BC052806.1 | cDNA clone MGC: 61802 IMAGE: 5730155 |
| BC053866.1 | endothelin 3, transcript variant 2 |
| BC054034.1 | U11/U12 snRNP 35K, transcript variant 2 |
| BC055314.1 | C2f protein |
| BC063275.1 | eukaryotic translation initiation factor 2C, 1, mRNA |
| CTL2110 | DNA TOPOISIMERASE(Scl-70) |
| CTL2112 | ssDNA |
| CTL2138 | RNP COMPLEX |
| CTL2142 | ssDNA |
| CTL2145 | RIBOSOMAL RNA |
| NM_000997.2 | ribosomal protein L37 (RPL37 |
| NM_001014.2 | ribosomal protein S10 (RPS10) |
| NM_001124.1 | adrenomedullin (ADM), mRNA |
| NM_001896.1 | casein kinase 2, alpha prime polypeptide (CSNK2A2) |
| NM_001896.2 | casein kinase 2, alpha prime polypeptide (CSNK2A2), mRNA |
| NM_002129.2 | high-mobility group box 2 (HMGB2), mRNA |
| NM_003516.2 | histone 2, H2aa (HIST2H2AA), mRNA |
| NM_004214.3 | fibroblast growth factor (acidic) intracellular binding protein (FIBP) |
| NM_004596.1 | Small nuclear ribonucleoprotein polypeptide A (SNRPA) |
| NM_004645.1 | coilin (COIL) |
| NM_006298.2 | zinc finger protein 192 (ZNF192), mRNA |
| NM_007285.5 | GABA(A) receptor-associated protein-like 2 (GABARAPL2) |
| NM_015488.1 | myofibrillogenesis regulator 1 (MR-1), |
| NM_015640.1 | PAI-1 mRNA-binding protein (PAI-RBP1) |
| NM_015987.2 | Heme binding protein 1 (HEBP1) |
| NM_016355.3 | DEAD (Asp-Glu-Ala-Asp) box polypeptide 47 (DDX47), transcript variant 1, |
| | mRNA |
| NM_016483.3 | PHD finger protein 7 (PHF7) |
| NM_016505.2 | putative S1 RNA binding domain protein (PS1D), mRNA |
| NM_016940.1 | chromosome 21 open reading frame 6 (C21orf6), mRNA |
| NM_018032.2 | LUC7-like (S. cerevisiae) (LUC7L) |
| NM_020239.2 | small protein effector 1 of Cdc42 (SPEC1) |
| NM_020661.1 | activation-induced cytidine deaminase (AICDA), mRNA |
| NM_032345.1 | PYM protein (PYM), mRNA |
| NM_054016.1 | FUS interacting protein (serine-arginine rich) 1 (FUSIP1), transcript variant |
| | 2, mRNA |
| NM_138775.1 | hypothetical protein BC015183 (LOC91801), mRNA |
| NM_144982.1 | hypothetical protein MGC23401 (MGC23401) |
| NM_152769.1 | chromosome 19 open reading frame 26 (C19orf26), mRNA |
| NM_153207.2 | AE binding protein 2 (AEBP2) |
| NM_153332.2 | 3′ exoribonuclease (3′HEXO), mRNA |

Table 5 is a list of autoantigens that were bound more often by antibodies in sera from individuals with ANCA than by antibodies in sera from healthy individuals. The normal count and ANCA count are presented along with the corresponding p-value.

TABLE 5
ANCA vs. healthy patients

| Genbank ID | | | | |
|---|---|---|---|---|
| number of | | | | |
| nucleic acid | | | | |
| coding for | Normal | ANCA | | |
| the protein | Count | Count | p-value | Name or description |
| BC000052.1 | 0 | 9 | 0.01174 | Similar to peroxisome proliferative activated receptor, |
| | | | | alpha |
| BC000103.1 | 3 | 17 | 0.004844 | NCK adaptor protein 2 |
| BC000442.1 | 1 | 13 | 0.005597 | serine/threonine kinase 12 |
| BC000914.1 | 0 | 12 | 0.001456 | splicing factor, arginine/serine-rich 3 |
| BC001120.1 | 10 | 11 | 0.01174 | lectin, galactoside-binding, soluble, 3 (galectin 3) |
| BC001371.2 | 0 | 9 | 0.01174 | chromosome 20 open reading frame 31, mRNA |
| BC001662.1 | 0 | 9 | 0.01174 | mitogen-activated protein kinase-activated protein |
| | | | | kinase 3 |
| BC002880.1 | 1 | 14 | 0.002599 | cysteinyl-tRNA synthetase |
| BC003168.1 | 0 | 9 | 0.01174 | oxysterol binding protein-like 10, |
| BC004514.1 | 1 | 12 | 0.011166 | hypothetical protein FLJ12584 |
| BC005332.1 | 0 | 11 | 0.003075 | cDNA clone MGC: 12418 IMAGE: 3934658, complete |
| | | | | cds |
| BC006105.1 | 2 | 14 | 0.013093 | chromosome 6 open reading frame 134, mRNA |
| BC007411.2 | 2 | 15 | 0.00624 | diaphanous homolog 1 (Drosophila) |
| BC007949.1 | 0 | 9 | 0.01174 | eukaryotic translation elongation factor 1 gamma |
| BC012876.1 | 0 | 14 | 0.000267 | clone MGC: 17259 IMAGE: 4149333 |
| BC013171.1 | 10 | 11 | 0.01174 | cDNA clone MGC: 17065 IMAGE: 4344401, complete |
| | | | | cds |
| BC014271.2 | 1 | 14 | 0.002599 | endoglin (Osler-Rendu-Weber syndrome 1), mRNA |
| BC014991.1 | 0 | 9 | 0.01174 | N-methylpurine-DNA glycosylase |
| BC015833.1 | 4 | 17 | 0.01839 | cDNA clone MGC: 27152 IMAGE: 4691630, complete |
| | | | | cds |
| BC016057.1 | 10 | 11 | 0.01174 | Usher syndrome 1C (autosomal recessive, severe), |
| | | | | mRNA |
| BC016312.1 | 10 | 11 | 0.01174 | chromosome 15 open reading frame 15, mRNA |
| BC016380.1 | 1 | 14 | 0.002599 | cDNA clone MGC: 27376 IMAGE: 4688477, complete |
| | | | | cds |
| BC016381.1 | 0 | 13 | 0.000647 | cDNA clone MGC: 27378 IMAGE: 4688865, complete |
| | | | | cds |
| BC016764.1 | 3 | 16 | 0.011617 | ribulose-5-phosphate-3-epimerase, transcript variant 1 |
| BC017865.1 | 0 | 11 | 0.003075 | Fc fragment of IgG, low affinity IIIa, receptor (CD16a), |
| | | | | mRNA |
| BC018302.1 | 0 | 9 | 0.01174 | TRM1 tRNA methyltransferase 1 homolog (S. cerevisiae), |
| | | | | mRNA |
| BC019337.1 | 4 | 19 | 0.002122 | immunoglobulin heavy constant gamma 1 (G1m |
| | | | | marker), mRNA |
| BC020622.1 | 1 | 13 | 0.005597 | zinc finger, A20 domain containing 1, mRNA, complete |
| | | | | cds. |
| BC020962.1 | 0 | 9 | 0.01174 | similar to glucosamine-6-sulfatases |
| BC022098.1 | 0 | 10 | 0.006149 | cDNA clone MGC: 31944 IMAGE: 4878869, complete |
| | | | | cds |
| BC022362.1 | 1 | 14 | 0.002599 | cDNA clone MGC: 23888 IMAGE: 4704496, complete |
| | | | | cds |
| BC024289.1 | 0 | 10 | 0.006149 | cDNA clone MGC: 39273 IMAGE: 5440834, complete |
| | | | | cds |
| BC025314.1 | 3 | 17 | 0.004844 | immunoglobulin heavy constant gamma 1 (G1m |
| | | | | marker), mRNA |
| BC025345.1 | 4 | 19 | 0.002122 | mRNA similar to LOC149651 (cDNA clone MGC: 39393 |
| | | | | IMAGE: 4862156), complete cds |
| BC029444.1 | 0 | 11 | 0.003075 | cDNA clone MGC: 32714 IMAGE: 4692138, complete |
| | | | | cds |
| BC029609.1 | 0 | 10 | 0.006149 | cDNA clone MGC: 39831 IMAGE: 5302675 |
| BC030590.1 | 1 | 12 | 0.011166 | retinoblastoma binding protein 8, mRNA |
| BC030814.1 | 0 | 14 | 0.000267 | immunoglobulin kappa variable 1-5, mRNA |
| BC030983.1 | 2 | 17 | 0.000974 | immunoglobulin lambda constant 1 (Mcg marker), |
| | | | | mRNA |
| BC030984.1 | 2 | 19 | 6.36E−05 | cDNA clone MGC: 32654 IMAGE: 4701898, complete |
| | | | | cds |
| BC031074.1 | 1 | 16 | 0.000414 | poly (ADP-ribose) polymerase family, member 16, |
| | | | | mRNA |
| BC032485.1 | 1 | 16 | 0.000414 | hypothetical protein FLJ30473 |
| BC032866.2 | 1 | 13 | 0.005597 | eukaryotic translation initiation factor 5, transcript |
| | | | | variant 2, mRNA |
| BC036075.1 | 0 | 9 | 0.01174 | GIPC PDZ domain containing family, member 2, mRNA |
| BC036723.1 | 1 | 12 | 0.011166 | Fc fragment of IgG, low affinity IIIa, receptor (CD16a), |
| | | | | mRNA |
| BC039814.1 | 1 | 17 | 0.000134 | zinc finger protein 265, transcript variant 2, mRNA |
| BC041157.1 | 0 | 11 | 0.003075 | thromboxane A synthase 1 (platelet, cytochrome P450, |
| | | | | family 5, subfamily A), transcript variant TXS-I, mRNA |
| BC044584.1 | 0 | 11 | 0.003075 | DnaJ (Hsp40) homolog, subfamily C, member 4, mRNA |
| BC053656.1 | 1 | 17 | 0.000134 | EGF-like repeats and discoidin I-like domains 3, mRNA |
| BC053664.1 | 0 | 12 | 0.001456 | complete cds. |
| BC053984.1 | 3 | 18 | 0.001657 | cDNA clone MGC: 59926 IMAGE: 5480266, complete |
| | | | | cds |
| BC056256.1 | 1 | 16 | 0.000414 | immunoglobulin kappa constant, mRNA |
| BC066938.1 | 4 | 17 | 0.01839 | DEAD (Asp-Glu-Ala-Asp) box polypeptide 43, mRNA |
| BC066987.1 | 0 | 9 | 0.01174 | cDNA clone MGC: 87634 IMAGE: 4838596, complete |
| | | | | cds |
| CTL1093 | 6 | 20 | 0.007663 | Human IgG |
| CTL2130 | 1 | 18 | 3.51E−05 | proteinase-3 |
| CTL2137 | 1 | 15 | 0.001099 | La/SS-B (La) |
| NM_001015.2 | 10 | 11 | 0.01174 | ribosomal protein S11 (RPS11) |
| NM_001663.2 | 2 | 16 | 0.002655 | ADP-ribosylation factor 6 (ARF6), mRNA |
| NM_001894.2 | 0 | 13 | 0.000647 | casein kinase 1, epsilon (CSNK1E) |
| NM_002019.1 | 10 | 11 | 0.01174 | fms-related tyrosine kinase 1 (vascular endothelial |
| | | | | growth factor/vascular permeability factor receptor) |
| | | | | (FLT1) |
| NM_002103.3 | 0 | 10 | 0.006149 | glycogen synthase 1 (muscle) (GYS1), mRNA |
| NM_002462.2 | 1 | 12 | 0.011166 | myxovirus (influenza virus) resistance 1, interferon- |
| | | | | inducible protein p78 (mouse) (MX1), mRNA |
| NM_003045.3 | 1 | 12 | 0.011166 | solute carrier family 7 (cationic amino acid transporter, |
| | | | | y+ system), member 1 (SLC7A1), mRNA |
| NM_003049.1 | 0 | 9 | 0.01174 | solute carrier family 10 (sodium/bile acid cotransporter |
| | | | | family), member 1 (SLC10A1), mRNA |
| NM_004055.3 | 3 | 17 | 0.004844 | calpain 5 (CAPN5), mRNA |
| NM_004217.1 | 0 | 10 | 0.006149 | aurora kinase B (AURKB) |
| NM_004732.1 | 4 | 17 | 0.01839 | potassium voltage-gated channel, shaker-related |
| | | | | subfamily, beta member 3 (KCNAB3) |
| NM_005522.3 | 0 | 11 | 0.003075 | homeo box A1 (HOXA1), transcript variant 1, mRNA |
| NM_006298.2 | 10 | 11 | 0.01174 | zinc finger protein 192 (ZNF192), mRNA |
| NM_006869.1 | 0 | 9 | 0.01174 | centaurin, alpha 1 (CENTA1), mRNA |
| NM_012163.1 | 0 | 10 | 0.006149 | F-box and leucine-rich repeat protein 9 (FBXL9) |
| NM_012241.2 | 1 | 13 | 0.005597 | sirtuin (silent mating type information regulation 2 |
| | | | | homolog) 5 (S. cerevisiae) (SIRT5), transcript variant 1, |
| | | | | mRNA |
| NM_016576.2 | 0 | 10 | 0.006149 | guanosine monophosphate reductase 2 (GMPR2) |
| NM_018047.1 | 0 | 9 | 0.01174 | RNA binding motif protein 22 (RBM22), mRNA |
| NM_018153.2 | 2 | 14 | 0.013093 | anthrax toxin receptor 1 (ANTXR1), transcript variant 3, |
| | | | | mRNA |
| NM_018184.1 | 0 | 10 | 0.006149 | ADP-ribosylation factor-like 10C (ARL10C) |
| NM_018679.2 | 0 | 10 | 0.006149 | t-complex 11 (mouse) (TCP11), mRNA |
| NM_019021.1 | 0 | 12 | 0.001456 | hypothetical protein FLJ20010 (FLJ20010), mRNA |
| NM_020367.2 | 0 | 9 | 0.01174 | chromosome 12 open reading frame 6 (C12orf6) |
| NM_020381.2 | 1 | 14 | 0.002599 | chromosome 6 open reading frame 210 (C6orf210), |
| | | | | mRNA |
| NM_020804.2 | 0 | 9 | 0.01174 | protein kinase C and casein kinase substrate in |
| | | | | neurons 1 (PACSIN1), mRNA |
| NM_021130.1 | 10 | 11 | 0.01174 | peptidylprolyl isomerase A (cyclophilin A) (PPIA) |
| NM_021133.1 | 3 | 16 | 0.011617 | ribonuclease L (2′,5′-oligoisoadenylate synthetase- |
| | | | | dependent) (RNASEL), |
| NM_021709.1 | 0 | 10 | 0.006149 | CD27-binding (Siva) protein (SIVA), transcript variant 2, |
| | | | | mRNA |
| NM_022787.2 | 4 | 17 | 0.01839 | nicotinamide nucleotide adenylyltransferase 1 |
| | | | | (NMNAT1), mRNA |
| NM_032855.1 | 0 | 12 | 0.001456 | hematopoietic SH2 protein (HSH2) |
| NM_052822.1 | 0 | 9 | 0.01174 | secretory carrier membrane protein 1 (SCAMP1), |
| | | | | transcript variant 2 |
| NM_052877.1 | 0 | 12 | 0.001456 | mediator of RNA polymerase II transcription, subunit 8 |
| | | | | homolog (yeast) (MED8) |
| NM_145315.2 | 10 | 11 | 0.01174 | lactation elevated 1 (LACE1) |
| NM_152688.1 | 0 | 10 | 0.006149 | KH domain containing, RNA binding, signal |
| | | | | transduction associated 2 (KHDRBS2), mRNA |
| NM_152697.2 | 3 | 16 | 0.011617 | hypothetical protein, mRNA |
| NM_153215.1 | 1 | 17 | 0.000134 | hypothetical protein FLJ38608 (FLJ38608), mRNA |
| NM_178496.2 | 1 | 13 | 0.005597 | similar to BcDNA: GH11415 gene product |
| | | | | (LOC151963), mRNA |
| NM_182665.1 | 0 | 12 | 0.001456 | Ras association (RalGDS/AF-6) domain family 5 |
| | | | | (RASSF5), transcript variant 3, mRNA |
| NM_198490.1 | 0 | 11 | 0.003075 | RAB43, member RAS oncogene family (RAB43), |
| | | | | mRNA |
| NM_203326.1 | 0 | 9 | 0.01174 | 5-azacytidine induced 2 (AZI2), transcript variant 2 |
| NM_212492.1 | 0 | 10 | 0.006149 | G protein pathway suppressor 1 (GPS1), transcript |
| | | | | variant 1, mRNA |

The autoantibodies listed in Table 6 are selective for ANCA, but not RA or SLE. Table 6 is a list of autoantibodies that were bound by an antibody from sera from an individual with ANCA but not healthy, RA or SLE patients.

TABLE 6
ANCA vs. all

| Genbank ID | |
|---|---|
| number of | |
| nucleic acid | |
| coding for | |
| the protein | Name or description |
| BC002880.1 | cysteinyl-tRNA synthetase |
| BC003168.1 | oxysterol binding protein-like 10, |
| BC005332.1 | cDNA clone MGC: 12418 IMAGE: 3934658, complete cds |
| BC006105.1 | chromosome 6 open reading frame 134, mRNA |
| BC020962.1 | similar to glucosamine-6-sulfatases |
| BC022098.1 | cDNA clone MGC: 31944 IMAGE: 4878869, complete cds |
| BC029444.1 | cDNA clone MGC: 32714 IMAGE: 4692138, complete cds |
| BC030814.1 | immunoglobulin kappa variable 1-5, mRNA |
| BC030983.1 | immunoglobulin lambda constant 1 (Mcg marker), mRNA |
| BC030984.1 | cDNA clone MGC: 32654 IMAGE: 4701898, complete cds |
| BC039814.1 | zinc finger protein 265, transcript variant 2, mRNA |
| BC044584.1 | DnaJ (Hsp40) homolog, subfamily C, member 4, mRNA |
| BC053664.1 | complete cds. |
| BC056256.1 | immunoglobulin kappa constant, mRNA |
| CTL2130 | proteinase-3 |
| CTL2137 | La/SS-B (La) |
| NM_004732.1 | potassium voltage-gated channel, shaker-related subfamily, beta |
| | member 3 (KCNAB3) |
| NM_006869.1 | centaurin, alpha 1 (CENTA1), mRNA |
| NM_012241.2 | sirtuin (silent mating type information regulation 2 homolog) 5 (S. cerevisiae) |
| | (SIRT5), transcript variant 1, mRNA |
| NM_020381.2 | chromosome 6 open reading frame 210 (C6orf210), mRNA |
| NM_052877.1 | mediator of RNA polymerase II transcription, subunit 8 homolog (yeast) |
| | (MED8) |
| NM_153215.1 | hypothetical protein FLJ38608 (FLJ38608), mRNA |

### Example 3

Serum from twelve individuals with RA prior to and following initiation of infliximab (Remicade®) treatment were profiled against a high throughput human protein array as described in Example 1. Table 7A is a list of autoantigens that were bound by antibodies from RA patient sera and showed a decrease count after twenty weeks of infliximab treatment. Table 7B is a list of autoantigens that were bound by antibodies from RA patient sera and showed an increase count after twenty weeks of infliximab treatment.

TABLE 7A
RA biomarkers showing a decrease count following treatment.

| Genbank ID | | | | |
|---|---|---|---|---|
| number of | | | | |
| nucleic acid | | | | |
| coding for the | | | | |
| protein | RA_T0 | RA_T20 | p-value | Name or description |
| BC012105.1 | 5 | 0 | 0.006192 | nuclear VCP-like, mRNA |
| BC025314.1 | 6 | 0 | 0.001548 | immunoglobulin heavy constant gamma 1 (G1m |
| | | | | marker), mRNA |
| BC028039.1 | 7 | 2 | 0.008454 | hypothetical protein MGC39900 |
| BC041037.1 | 6 | 1 | 0.008978 | immunoglobulin heavy constant mu, mRNA |
| NM_003848.1 | 5 | 0 | 0.006192 | succinate-CoA ligase, GDP-forming, beta subunit |
| | | | | (SUCLG2), mRNA |
| NM_020367.2 | 6 | 1 | 0.008978 | chromosome 12 open reading frame 6 (C12orf6) |
| NM_133484.1 | 5 | 0 | 0.006192 | TRAF family member-associated NFKB activator |
| | | | | (TANK), transcript variant 2, mRNA |

TABLE 7B
RA biomarkers showing an increase count following treatment.

| Genbank ID | | | | |
|---|---|---|---|---|
| number of | | | | |
| nucleic acid | | | | |
| coding for | | | | |
| the protein | RA_T0 | RA_T20 | p-value | Name or description |
| BC001132.1 | 0 | 6 | 0.017028 | DEAD (Asp-Glu-Ala-Asp) box polypeptide 54 |
| BC005382.1 | 3 | 11 | 0.008978 | SPANX family, member E, mRNA |
| BC006550.1 | 4 | 12 | 0.006192 | RNA binding motif protein, X chromosome |
| BC009894.2 | 4 | 12 | 0.006192 | 3′-phosphoadenosine 5′-phosphosulfate synthase 2 |
| BC011792.1 | 0 | 6 | 0.017028 | Clone MGC: 19561 IMAGE: 4300082 |
| BC016609.1 | 1 | 10 | 0.001718 | cytidine monophosphate N-acetylneuraminic acid |
| | | | | synthetase, mRNA |
| BC034247.1 | 0 | 6 | 0.017028 | chromosome 9 open reading frame 105, mRNA |
| BC053557.1 | 0 | 6 | 0.017028 | cDNA clone MGC: 61706 IMAGE: 6162269 |
| BC064367.1 | 0 | 6 | 0.017028 | sterile alpha motif domain containing 6, mRNA |
| NM_000594.2 | 0 | 10 | 0.000187 | tumor necrosis factor (TNF superfamily, member 2) |
| | | | | (TNF), mRNA |
| NM_001449.2 | 1 | 8 | 0.015905 | four and a half LIM domains 1 (FHL1) |
| NM_004217.1 | 1 | 8 | 0.015905 | aurora kinase B (AURKB) |
| NM_005926.2 | 1 | 10 | 0.001718 | microfibrillar-associated protein 1 (MFAP1), mRNA |
| NM_012101.2 | 0 | 7 | 0.006811 | tripartite motif-containing 29 (TRIM29), transcript |
| | | | | variant 1, mRNA |
| NM_058163.1 | 0 | 7 | 0.006811 | hypothetical protein DT1P1A10 (DT1P1A10), |
| | | | | mRNA |
| NM_183241.1 | 0 | 6 | 0.017028 | hypothetical protein LOC286257 (LOC286257), |
| | | | | mRNA |

### Example 4

Serum samples from individuals with autoimmune diseases including RA (Rheumatoid Arthritis), SLE (Systemic Lupus Erythrematosus) and ANCA (Anti-Neutrophil Cytoplasmic Antibody) were profiled on ProtoArray™ human protein microarrays as described in Example 1. Utilizing the calculations as described below, the antigen biomarkers for each autoimmune disease were compared with one another to identify biomarkers selective for each particular disease. The tables below identify the autoantigens which are present for one autoimmune disease, such as RA, SLE, and ANCA, but are not present for another disease.

Tables 8-13 identify antigens according to Genbank ID number for the nucleotide sequence that encodes the antigens. It is understood that an antigen of Tables 8-13 refers to a protein or fragments thereof that is encoded by the nucleotide sequence associated with the nucleotide ID number. Table 8 lists antigens that were bound by an antibody from RA patient sera but not by an antibody from SLE patient sera. Table 9 lists antigens that were bound by an antibody from RA patient sera but not by an antibody from ANCA patient sera. Table 10 lists antigens that were bound by an antibody from SLE patient sera but not by an antibody from RA patient sera. Table 11 lists antigens that were bound by an antibody from SLE patient sera but not by an antibody from ANCA patient sera. Table 12 lists antigens that were bound by an antibody from ANCA patient sera but not by an antibody from SLE patient sera. Table 13 lists antigens that were bound by an antibody from ANCA patient sera but not by an antibody from RA patient sera.

TABLE 8
Table 8 is a list of proteins that were bound by an antibody from RA patient
sera but not SLE patients.

| Genbank ID | | | | |
|---|---|---|---|---|
| number of | | | | |
| nucleic acid | | | | |
| coding for | RA | SLE | | |
| the protein | Count | Count | p-value | Name or description |
| BC001120.1 | 8 | 4 | 0.000862 | lectin, galactoside-binding, soluble, 3 (galectin 3) |
| BC001286.1 | 5 | 0 | 0.001061 | dCMP deaminase, mRNA |
| BC001694.1 | 8 | 7 | 0.009495 | clone MGC: 2299 IMAGE: 2967519 |
| BC005332.1 | 4 | 0 | 0.005305 | cDNA clone MGC: 12418 IMAGE: 3934658, |
| | | | | complete cds |
| BC012105.1 | 8 | 7 | 0.009495 | nuclear VCP-like, mRNA |
| BC012576.1 | 8 | 7 | 0.009495 | Unknown (protein for MGC: 13472) |
| BC012876.1 | 4 | 0 | 0.005305 | clone MGC: 17259 IMAGE: 4149333 |
| BC014271.2 | 4 | 0 | 0.005305 | endoglin (Osler-Rendu-Weber syndrome 1), mRNA |
| BC014435.1 | 8 | 5 | 0.002128 | Unknown (protein for MGC: 22922) |
| BC016380.1 | 4 | 0 | 0.005305 | cDNA clone MGC: 27376 IMAGE: 4688477, |
| | | | | complete cds |
| BC016381.1 | 6 | 1 | 0.001099 | cDNA clone MGC: 27378 IMAGE: 4688865, |
| | | | | complete cds |
| BC018111.1 | 9 | 8 | 0.002427 | pim-2 oncogene |
| BC019337.1 | 5 | 0 | 0.001061 | immunoglobulin heavy constant gamma 1 (G1m |
| | | | | marker), mRNA |
| BC022098.1 | 9 | 7 | 0.001142 | cDNA clone MGC: 31944 IMAGE: 4878869, |
| | | | | complete cds |
| BC022429.1 | 5 | 2 | 0.016438 | cDNA clone MGC: 24679 IMAGE: 4270959, |
| | | | | complete cds |
| BC024289.1 | 7 | 1 | 0.00017 | cDNA clone MGC: 39273 IMAGE: 5440834, |
| | | | | complete cds |
| BC025314.1 | 6 | 0 | 0.00177 | immunoglobulin heavy constant gamma 1 (G1m |
| | | | | marker), mRNA |
| BC028039.1 | 8 | 7 | 0.009495 | hypothetical protein MGC39900 |
| BC029444.1 | 6 | 2 | 0.003888 | cDNA clone MGC: 32714 IMAGE: 4692138, |
| | | | | complete cds |
| BC032451.1 | 7 | 2 | 0.000701 | cDNA clone MGC: 40426 IMAGE: 5178085, |
| | | | | complete cds |
| BC033195.1 | 4 | 0 | 0.005305 | hypothetical gene FLJ00060 |
| BC041037.1 | 7 | 0 | 2.31E−05 | immunoglobulin heavy constant mu, mRNA |
| BC051885.1 | 7 | 4 | 0.005343 | chromosome 14 open reading frame 106, mRNA, |
| | | | | complete cds. |
| BC053984.1 | 6 | 0 | 0.000177 | cDNA clone MGC: 59926 IMAGE: 5480266, |
| | | | | complete cds |
| CTL2131 | 5 | 2 | 0.016438 | CCP peptide 1% BSA_1 mg/ml |
| CTL2134 | 9 | 7 | 0.001142 | dsDNA |
| NM_000431.1 | 5 | 2 | 0.016438 | mevalonate kinase (mevalonic aciduria) (MVK), |
| | | | | mRNA |
| NM_001157.2 | 5 | 2 | 0.016438 | annexin A11 (ANXA11), transcript variant a, mRNA |
| NM_001667.1 | 8 | 7 | 0.009495 | ADP-ribosylation factor-like 2 (ARL2), mRNA |
| NM_002476.2 | 9 | 7 | 0.001142 | myosin, light polypeptide 4, alkali; atrial, embryonic |
| | | | | (MYL4) |
| NM_002963.2 | 9 | 8 | 0.002427 | S100 calcium binding protein A7 (psoriasin 1) |
| | | | | (S100A7), mRNA |
| NM_004722.2 | 8 | 7 | 0.009495 | adaptor-related protein complex 4, mu 1 subunit |
| | | | | (AP4M1), mRNA |
| NM_005435.2 | 7 | 5 | 0.011617 | Rho guanine nucleotide exchange factor (GEF) 5 |
| | | | | (ARHGEF5) |
| NM_005697.3 | 6 | 1 | 0.001099 | secretory carrier membrane protein 2 (SCAMP2), |
| | | | | mRNA |
| NM_006002.2 | 9 | 7 | 0.001142 | ubiquitin carboxyl-terminal esterase L3 (ubiquitin |
| | | | | thiolesterase) (UCHL3), |
| NM_006169.1 | 5 | 2 | 0.016438 | nicotinamide N-methyltransferase (NNMT) |
| NM_013322.2 | 8 | 6 | 0.004698 | sorting nexin 10 (SNX10), mRNA |
| NM_016207.2 | 8 | 6 | 0.004698 | cleavage and polyadenylation specific factor 3, |
| | | | | 73 kDa (CPSF3), mRNA |
| NM_019023.1 | 8 | 7 | 0.009495 | protein arginine N-methyltransferase 7 (PRMT7), |
| | | | | mRNA |
| NM_024610.2 | 8 | 7 | 0.009495 | HSPB (heat shock 27 kDa) associated protein 1 |
| | | | | (HSPBAP1) |
| NM_032781.2 | 8 | 7 | 0.009495 | protein tyrosine phosphatase, non-receptor type 5 |
| | | | | (striatum-enriched) (PTPN5), mRNA |
| NM_033064.1 | 8 | 7 | 0.009495 | ataxia, cerebellar, Cayman type (caytaxin) |
| | | | | (ATCAY) |
| NM_033161.2 | 8 | 6 | 0.004698 | surfeit 4 (SURF4), mRNA |
| NM_080876.2 | 8 | 7 | 0.009495 | dual specificity phosphatase 19 (DUSP19), mRNA |
| NM_152653.1 | 4 | 0 | 0.005305 | ubiquitin-conjugating enzyme E2E 2 (UBC4/5 |
| | | | | homolog, yeast) (UBE2E2), mRNA |

TABLE 9
Table 9 is a list of proteins that were bound by an antibody from RA patient
sera but not ANCA patients.

| Genbank ID | | | | |
|---|---|---|---|---|
| number of | | | | |
| nucleic acid | | | | |
| coding for | RA | ANCA | | |
| the protein | Count | Count | p-value | Name or description |
| BC001120.1 | 9 | 8 | 0.016771 | lectin, galactoside-binding, soluble, 3 (galectin 3 |
| BC001286.1 | 4 | 0 | 0.005305 | dCMP deaminase, mRNA |
| BC007347.2 | 4 | 0 | 0.005305 | Unknown (protein for MGC: 1566) |
| BC008715.2 | 4 | 0 | 0.005305 | microtubule-associated protein 4, mRNA |
| BC010697.1 | 6 | 3 | 0.010263 | amylase, alpha 2B; pancreatic |
| BC013567.1 | 4 | 0 | 0.005305 | hypothetical protein FLJ11328 |
| BC014218.2 | 4 | 0 | 0.005305 | cDNA clone IMAGE: 3954254 |
| BC017570.1 | 4 | 0 | 0.005305 | chromosome 9 open reading frame 78, mRNA |
| BC031281.1 | 4 | 0 | 0.005305 | tetratricopeptide repeat domain 16, mRNA |
| BC032334.1 | 5 | 0 | 0.001061 | putative homeodomain transcription factor 2, |
| | | | | mRNA, complete cds. |
| BC033195.1 | 6 | 2 | 0.003888 | hypothetical gene FLJ00060 |
| BC036107.1 | 5 | 1 | 0.016438 | heat shock 70 kDa protein 2, mRNA |
| CTL2132 | 6 | 1 | 0.001099 | myeloperoxidase_100 ug/ml_S |
| NM_002476.2 | 4 | 0 | 0.005305 | myosin, light polypeptide 4, alkali; atrial, embryonic |
| NM_002540.3 | 6 | 3 | 0.010263 | outer dense fiber of sperm tails 2 (ODF2), transcript |
| | | | | variant 1, mRNA |
| NM_003576.2 | 9 | 4 | 0.016771 | serine/threonine kinase 24 (STE20 homolog, yeast) |
| NM_003662.1 | 4 | 0 | 0.005305 | Pirin (PIR) |
| NM_003691.1 | 8 | 7 | 0.009495 | serine/threonine kinase 16 (STK16) |
| NM_006169.1 | 5 | 0 | 0.001061 | nicotinamide N-methyltransferase (NNMT) |
| NM_012425.2 | 4 | 0 | 0.005305 | Ras suppressor protein 1 (RSU1) |
| NM_016520.1 | 4 | 0 | 0.005305 | chromosome 9 open reading frame 78 (C9orf78), |
| | | | | mRNA |
| NM_022822.1 | 4 | 0 | 0.005305 | likely ortholog of kinesin light chain 2 (KLC2), mRNA |
| NM_024053.1 | 5 | 1 | 0.016438 | chromosome 22 open reading frame 18 (C22orf18) |
| NM_024779.2 | 9 | 5 | 0.002427 | hypothetical protein, mRNA |
| NM_032781.2 | 8 | 7 | 0.017848 | hypothetical protein (FLJ32384), mRNA |
| NM_033064.1 | 5 | 2 | 0.016438 | hypothetical protein, mRNA |
| NM_052848.1 | 4 | 0 | 0.005305 | ubiquitin-conjugating enzyme E2E 2 (UBC4/5 |
| | | | | homolog, yeast) (UBE2E2), mRNA |
| NM_144608.1 | 4 | 0 | 0.005305 | Meis1, myeloid ecotropic viral integration site 1 |
| | | | | homolog 2 (mouse) (MEIS2), transcript variant d, |
| | | | | mRNA |
| NM_152362.1 | 6 | 1 | 0.003888 | hypothetical protein, mRNA |
| NM_152653.1 | 4 | 0 | 0.005305 | ubiquitin-conjugating enzyme E2E 2 (UBC4/5 |
| | | | | homolog, yeast) (UBE2E2) |
| NM_170676.2 | 5 | 2 | 0.016438 | Meis1, myeloid ecotropic viral integration site 1 |
| | | | | homolog 2 (mouse) (MEIS2), transcript variant d |
| NM_173519.1 | 5 | 1 | 0.005482 | hypothetical protein MGC34646 (MGC34646) |

TABLE 10
Table 10 is a list of proteins that were bound by an antibody from SLE patient
sera but not RA patients.

| Genbank ID | | | | |
|---|---|---|---|---|
| number of | | | | |
| nucleic acid | | | | |
| coding for | SLE | RA | | |
| the protein | Count | Count | p-value | Name or description |
| BC000084.1 | 0 | 9 | 0.016771 | hypothetical protein FLJ10357 |
| BC000238.1 | 0 | 9 | 0.016771 | hypothetical protein FLJ10415, mRNA |
| BC000381.2 | 0 | 10 | 0.009224 | TBP-like 1, mRNA |
| BC000442.1 | 0 | 10 | 0.009224 | serine/threonine kinase 12 |
| BC000463.1 | 0 | 13 | 0.001142 | splicing factor 3b, subunit 3, 130 kD |
| BC000557.1 | 9 | 11 | 0.016771 | phosphatidylethanolamine N-methyltransferase |
| BC000691.1 | 9 | 10 | 0.009224 | brain specific protein |
| BC000877.1 | 1 | 14 | 0.004698 | vasopressin-induced transcript |
| BC000921.2 | 0 | 10 | 0.009224 | methyltransferase like 5, mRNA |
| BC000979.2 | 9 | 10 | 0.009224 | DEAD (Asp-Glu-Ala-Asp) box polypeptide 49, |
| BC001280.1 | 0 | 11 | 0.004855 | serine/threonine kinase 6, transcript variant 1 |
| BC001280.1 | 0 | 9 | 0.016771 | serine/threonine kinase 6, transcript variant 1 |
| BC001294.1 | 0 | 9 | 0.016771 | Similar to x 006 protein |
| BC001396.1 | 0 | 10 | 0.009224 | AD-003 protein, clone MGC: 783 IMAGE: 3050940 |
| BC002509.1 | 0 | 9 | 0.016771 | clone MGC: 2941 IMAGE: 3051214 |
| BC002606.1 | 0 | 9 | 0.016771 | Similar to hypothetical protein, clone MGC: 2992 |
| | | | | IMAGE: 3160695 |
| BC002733.2 | 3 | 17 | 0.010263 | mRNA, complete cds. |
| BC003360.1 | 0 | 11 | 0.004855 | DEAD (Asp-Glu-Ala-Asp) box polypeptide 18, |
| | | | | mRNA |
| BC004301.1 | 0 | 9 | 0.016771 | core promoter element binding protein, mRNA |
| BC005004.1 | 0 | 10 | 0.009224 | family with sequence similarity 64, member A, |
| | | | | mRNA |
| BC005955.1 | 0 | 10 | 0.009224 | hypothetical protein MGC14595 |
| BC006376.1 | 0 | 10 | 0.009224 | N-myristoyltransferase 2, clone MGC: 12700 |
| BC006550.1 | 0 | 12 | 0.002427 | RNA binding motif protein, X chromosome |
| BC007320.2 | 9 | 11 | 0.016771 | annexin A10 |
| BC007833.2 | 1 | 14 | 0.004698 | phosphatidylinositol-4-phosphate 5-kinase, type I, |
| | | | | alpha, mRNA |
| BC008077.2 | 0 | 11 | 0.004855 | signal recognition particle receptor (‘docking |
| | | | | protein’), mRNA |
| BC008623.1 | 0 | 10 | 0.009224 | hypothetical protein FLJ21044 similar to Rbig1, |
| | | | | cloneMGC: 16823 IMAGE: 4177689, mRNA, |
| | | | | complete cds. |
| BC009294.1 | 0 | 9 | 0.016771 | clone MGC: 16644 IMAGE: 4123062 |
| BC009348.2 | 0 | 9 | 0.016771 | cirrhosis, autosomal recessive 1A (cirhin), mRNA |
| BC009623.1 | 0 | 15 | 0.0002 | Similar to nucleophosmin (nucleolar phosphoprotein |
| | | | | B23, numatrin) |
| BC009762.2 | 0 | 12 | 0.002427 | mRNA, complete cds. |
| BC009819.1 | 0 | 10 | 0.009224 | hypothetical protein FLJ23591 |
| BC009829.1 | 0 | 13 | 0.001142 | hypothetical protein FLJ11526 |
| BC009894.2 | 5 | 20 | 0.005305 | 3′-phosphoadenosine 5′-phosphosulfate synthase 2 |
| BC010074.2 | 0 | 11 | 0.004855 | FUS interacting protein (serine/arginine-rich) 1, |
| | | | | mRNA |
| BC010176.1 | 9 | 11 | 0.016771 | clone MGC: 20533 IMAGE: 3342874 |
| BC010467.1 | 1 | 14 | 0.004698 | cDNA clone MGC: 17410 IMAGE: 4156035 |
| BC010501.1 | 0 | 11 | 0.004855 | catenin (cadherin-associated protein), delta 1 |
| BC010642.1 | 0 | 9 | 0.016771 | zinc finger protein 22 (KOX 15) |
| BC010947.1 | 0 | 9 | 0.016771 | signal recognition particle 19 kDa, mRNA |
| BC011498.1 | 1 | 13 | 0.009495 | Unknown (protein for MGC: 17017) |
| BC011668.1 | 0 | 10 | 0.009224 | Similar to casein kinase 2, alpha 1 polypeptide |
| BC011792.1 | 0 | 13 | 0.001142 | clone MGC: 19561 IMAGE: 4300082 |
| BC011842.2 | 0 | 13 | 0.001142 | hypothetical protein FLJ11184, mRNA |
| BC011885.1 | 9 | 10 | 0.009224 | eukaryotic translation initiation factor (elF) 2A, |
| | | | | mRNA |
| BC012472.1 | 1 | 17 | 0.000302 | ubiquitin D, mRNA |
| BC012566.1 | 0 | 9 | 0.016771 | nucleophosmin (nucleolar phosphoprotein B23, |
| | | | | numatrin), mRNA |
| BC012865.1 | 0 | 11 | 0.004855 | retinoic acid induced 16 |
| BC013073.1 | 0 | 10 | 0.009224 | chromosome 1 open reading frame 37, mRNA |
| BC013319.1 | 0 | 9 | 0.016771 | Similar to hypothetical protein FLJ11183, |
| | | | | cloneMGC: 13390 IMAGE: 4286103, mRNA, |
| | | | | complete cds. |
| BC013900.1 | 0 | 9 | 0.016771 | hypothetical protein FLJ20436 |
| BC013966.2 | 0 | 10 | 0.009224 | family with sequence similarity 64, member A, |
| | | | | mRNA |
| BC014441.1 | 0 | 11 | 0.004855 | NOL1/NOP2/Sun domain family, member 4, mRNA |
| BC014452.1 | 0 | 10 | 0.009224 | cDNA clone IMAGE: 4903661 |
| BC014949.1 | 0 | 9 | 0.016771 | likely ortholog of mouse D11Igp2, mRNA |
| BC014991.1 | 4 | 18 | 0.016438 | N-methylpurine-DNA glycosylase |
| BC015008.1 | 1 | 16 | 0.000862 | hydroxyacylglutathione hydrolase-like, mRNA |
| BC015497.1 | 0 | 11 | 0.004855 | cDNA clone MGC: 9014 IMAGE: 3913870, complete |
| | | | | cds |
| BC015569.1 | 0 | 9 | 0.016771 | Similar to SRp25 nuclear protein |
| BC015715.1 | 1 | 13 | 0.009495 | makorin, ring finger protein, 2 |
| BC016276.1 | 0 | 11 | 0.004855 | KIAA0008 gene product, clone MGC: 768 |
| | | | | IMAGE: 3537754 |
| BC016609.1 | 1 | 12 | 0.017848 | cytidine monophosphate N-acetylneuraminic acid |
| | | | | synthetase, mRNA |
| BC016764.1 | 3 | 20 | 0.000177 | ribulose-5-phosphate-3-epimerase |
| BC016768.1 | 0 | 9 | 0.016771 | nucleophosmin (nucleolar phosphoprotein B23, |
| | | | | numatrin), mRNA |
| BC016842.1 | 0 | 9 | 0.016771 | family with sequence similarity 61, member A, |
| | | | | mRNA |
| BC017114.1 | 0 | 14 | 0.0005 | hypothetical protein FLJ22833 |
| BC017212.2 | 0 | 9 | 0.016771 | PHD finger protein 11, mRNA |
| BC017296.2 | 9 | 11 | 0.016771 | sestrin 3, mRNA |
| BC017943.1 | 0 | 9 | 0.016771 | protein phosphatase 1 regulatory subunit 1A |
| BC018630.1 | 0 | 9 | 0.016771 | Similar to KIAA0471 gene product, clone |
| | | | | MGC: 32006 IMAGE: 4308560 |
| BC018749.1 | 9 | 11 | 0.016771 | immunoglobulin lambda variable 2-14, mRNA |
| BC018823.2 | 2 | 15 | 0.011617 | splicing factor, arginine/serine-rich 5 |
| BC019598.1 | 0 | 10 | 0.009224 | zinc finger, matrin type 4, mRNA |
| BC020597.1 | 0 | 10 | 0.009224 | general transcription factor IIB |
| BC020647.1 | 0 | 10 | 0.009224 | HSPC128 protein, mRNA |
| BC020962.1 | 2 | 16 | 0.005343 | similar to glucosamine-6-sulfatases |
| BC021121.1 | 3 | 20 | 0.000177 | protein kinase, lysine deficient 1 |
| BC021263.1 | 9 | 11 | 0.016771 | RAB24, member RAS oncogene family |
| BC021282.1 | 1 | 13 | 0.009495 | zinc finger protein 444 |
| BC021930.1 | 0 | 10 | 0.009224 | Unknown (protein for MGC: 32072) |
| BC021983.1 | 1 | 15 | 0.002128 | nucleophosmin (nucleolar phosphoprotein B23, |
| | | | | numatrin), transcript variant 1, mRNA |
| BC022077.1 | 2 | 15 | 0.011617 | hypothetical protein MGC33338 |
| BC022325.1 | 1 | 17 | 0.000302 | hypothetical protein FLJ12729 |
| BC022361.1 | 0 | 9 | 0.016771 | chromosome 14 open reading frame 111 |
| BC024184.2 | 0 | 11 | 0.004855 | germ cell-less homolog 1 (Drosophila)-like, mRNA |
| BC025996.2 | 0 | 9 | 0.016771 | cDNA clone MGC: 26787 IMAGE: 4838986 |
| BC026104.2 | 0 | 9 | 0.016771 | programmed cell death 4 (neoplastic transformation |
| | | | | inhibitor), transcript variant 1, mRNA |
| BC027607.1 | 0 | 10 | 0.009224 | clone MGC: 26892 IMAGE: 4828241, |
| BC028040.1 | 0 | 15 | 0.0002 | 2′,3′-cyclic nucleotide 3′ phosphodiesterase, mRNA |
| BC028301.1 | 1 | 14 | 0.004698 | mRNA similar to LOC147447 |
| BC028672.1 | 0 | 9 | 0.016771 | chromosome 15 open reading frame 15, mRNA |
| BC028711.2 | 0 | 13 | 0.001142 | hypothetical protein MGC27005 |
| BC029046.1 | 1 | 12 | 0.017848 | H1 histone family, member 0, mRNA |
| BC029406.1 | 9 | 11 | 0.016771 | angiopoietin 1, mRNA, complete cds. |
| BC029775.1 | 0 | 9 | 0.016771 | hypothetical protein LOC199964 |
| BC029891.1 | 0 | 10 | 0.009224 | transcription factor EC, mRNA |
| BC030702.1 | 0 | 13 | 0.001142 | hypothetical protein FLJ12847 |
| BC030711.2 | 1 | 13 | 0.009495 | chromosome 2 open reading frame 13 |
| BC030783.1 | 0 | 11 | 0.004855 | glycerol-3-phosphate acyltransferase, mitochondrial, |
| | | | | mRNA |
| BC031682.1 | 1 | 12 | 0.017848 | hypothetical protein MGC10433, mRNA |
| BC032347.1 | 0 | 11 | 0.004855 | chromosome 8 open reading frame 59, mRNA |
| BC032852.2 | 3 | 17 | 0.010263 | melanoma antigen family B, 4, mRNA |
| BC033242.1 | 1 | 12 | 0.017848 | methyl-CpG binding domain protein 1, transcript |
| | | | | variant 3, mRNA |
| BC033621.2 | 0 | 12 | 0.002427 | hypothetical protein DKFZp434G1415, mRNA |
| BC033758.1 | 0 | 10 | 0.009224 | centaurin, alpha 2, mRNA |
| BC033856.1 | 0 | 11 | 0.004855 | Similar to RIKEN cDNA 3110040D16 gene, |
| | | | | cloneMGC: 45395 IMAGE: 5123380, mRNA, |
| | | | | complete cds. |
| BC034236.1 | 9 | 11 | 0.016771 | hypothetical protein MGC39821 |
| BC034401.1 | 9 | 10 | 0.009224 | Similar to LOC161981 |
| BC035314.1 | 0 | 9 | 0.016771 | brix domain containing 1 |
| BC036365.1 | 0 | 12 | 0.002427 | hypothetical protein FLJ23537 |
| BC038105.2 | 2 | 17 | 0.002135 | membrane protein, palmitoylated 7 (MAGUK p55 |
| | | | | subfamily member 7) |
| BC038808.1 | 0 | 12 | 0.002427 | apolipoprotein B mRNA editing enzyme, catalytic |
| | | | | polypeptide-like 3F, transcript variant 1, mRNA |
| BC039711.1 | 0 | 11 | 0.004855 | translokin, mRNA |
| BC040177.2 | 1 | 14 | 0.004698 | protein phosphatase 1H (PP2C domain containing), |
| | | | | mRNA |
| BC042625.1 | 1 | 15 | 0.002128 | LUC7-like 2 (S. cerevisiae), mRNA |
| BC044953.1 | 1 | 15 | 0.002128 | zinc finger protein 620 |
| BC045535.1 | 0 | 9 | 0.016771 | chromosome 1 open reading frame 25, mRNA |
| BC047472.1 | 9 | 11 | 0.016771 | mRNA similar to RIKEN cDNA 4832415H08 gene, |
| | | | | complete cds. |
| BC050428.1 | 0 | 13 | 0.001142 | katanin p60 (ATPase-containing) subunit A 1, |
| | | | | mRNA |
| BC050563.1 | 2 | 15 | 0.011617 | hypothetical protein LOC202051, mRNA |
| BC050603.1 | 0 | 12 | 0.002427 | hypothetical protein MGC3329, mRNA |
| BC051790.1 | 0 | 10 | 0.009224 | mRNA similar to hypothetical protein FLJ20651 |
| | | | | (cDNAclone MGC: 57594 IMAGE: 6190506), |
| | | | | complete cds. |
| BC052806.1 | 0 | 11 | 0.004855 | cDNA clone MGC: 61802 IMAGE: 5730155 |
| BC053365.1 | 0 | 9 | 0.016771 | ribosomal protein S6 kinase, 70 kDa, polypeptide 1, |
| | | | | mRNA |
| BC053557.1 | 0 | 10 | 0.009224 | cDNA clone MGC: 61706 IMAGE: 6162269 |
| BC053866.1 | 3 | 17 | 0.010263 | endothelin 3, transcript variant 2 |
| BC054031.2 | 0 | 11 | 0.004855 | mitochondrial ribosomal protein S17 |
| BC054034.1 | 1 | 14 | 0.004698 | U11/U12 snRNP 35K |
| BC054892.1 | 9 | 11 | 0.016771 | dynein, cytoplasmic, light polypeptide 2B, mRNA |
| BC055314.1 | 1 | 14 | 0.004698 | C2f protein |
| BC056148.1 | 0 | 10 | 0.009224 | nuclear receptor subfamily 1, group D, member 1 |
| BC057809.1 | 0 | 9 | 0.016771 | complete cds. |
| BC061699.1 | 0 | 10 | 0.009224 | glycosyltransferase-like domain containing 1, mRNA |
| BC063275.1 | 0 | 14 | 0.0005 | eukaryotic translation initiation factor 2C, 1, mRNA |
| BC063463.1 | 9 | 10 | 0.009224 | coenzyme Q3 homolog, methyltransferase (yeast), |
| | | | | mRNA |
| BC064367.1 | 0 | 9 | 0.016771 | sterile alpha motif domain containing 6, mRNA |
| BC064841.1 | 0 | 9 | 0.016771 | complete cds. |
| CTL2110 | 1 | 15 | 0.002128 | DNA TOPOISIMERASE(Scl-70) |
| CTL2112 | 0 | 13 | 0.001142 | ssDNA |
| CTL2138 | 0 | 15 | 0.0002 | RNP COMPLEX |
| CTL2142 | 1 | 14 | 0.004698 | ssDNA |
| CTL2144 | 9 | 11 | 0.016771 | TRANSGLUTAMINASE |
| CTL2145 | 0 | 13 | 0.001142 | RIBOSOMAL RNA |
| CTL2150 | 1 | 14 | 0.004698 | SMITH ANTIGEN |
| CTL2151 | 0 | 9 | 0.016771 | Ro-52 |
| NM_000107.1 | 0 | 10 | 0.009224 | damage-specific DNA binding protein 2, 48 kDa |
| | | | | (DDB2), mRNA |
| NM_000327.2 | 9 | 11 | 0.016771 | retinal outer segment membrane protein 1 (ROM1), |
| | | | | mRNA |
| NM_000723.3 | 2 | 15 | 0.011617 | calcium channel, voltage-dependent, beta 1 subunit |
| | | | | (CACNB1), transcript variant 1, mRNA |
| NM_000970.2 | 0 | 10 | 0.009224 | ribosomal protein L6 (RPL6) |
| NM_000975.2 | 1 | 15 | 0.002128 | ribosomal protein L11 (RPL11), mRNA |
| NM_000984.2 | 1 | 12 | 0.017848 | ribosomal protein L23a (RPL23A) |
| NM_000993.2 | 0 | 9 | 0.016771 | ribosomal protein L31 (RPL31), mRNA |
| NM_000997.2 | 0 | 10 | 0.009224 | ribosomal protein L37 (RPL37) |
| NM_001002913.1 | 0 | 11 | 0.004855 | chromosome 9 open reading frame 115 (C9orf115), |
| | | | | mRNA |
| NM_001013.2 | 0 | 14 | 0.0005 | ribosomal protein S9 (RPS9) |
| NM_001014.2 | 0 | 15 | 0.0002 | ribosomal protein S10 (RPS10) |
| NM_001022.3 | 1 | 16 | 0.000862 | ribosomal protein S19 (RPS19), mRNA |
| NM_001023.2 | 0 | 9 | 0.016771 | ribosomal protein S20 (RPS20), mRNA |
| NM_001029.2 | 0 | 9 | 0.016771 | ribosomal protein S26 (RPS26) |
| NM_001124.1 | 0 | 12 | 0.002427 | adrenomedullin (ADM), mRNA |
| NM_001203.1 | 0 | 11 | 0.004855 | bone morphogenetic protein receptor, type IB |
| | | | | (BMPR1B), mRNA |
| NM_001280.1 | 1 | 13 | 0.009495 | cold inducible RNA binding protein (CIRBP), mRNA |
| NM_001626.2 | 9 | 11 | 0.016771 | v-akt murine thymoma viral oncogene homolog 2 |
| | | | | (AKT2) |
| NM_001662.2 | 0 | 9 | 0.016771 | ADP-ribosylation factor 5 (ARF5), mRNA |
| NM_001697.1 | 0 | 15 | 0.0002 | ATP synthase, H+ transporting, mitochondrial F1 |
| | | | | complex, O subunit (oligomycin sensitivity |
| | | | | conferring protein) (ATP5O) |
| NM_001759.2 | 9 | 10 | 0.009224 | cyclin D2 (CCND2), mRNA |
| NM_001894.2 | 0 | 12 | 0.002427 | casein kinase 1, epsilon (CSNK1E) |
| NM_001896.1 | 3 | 17 | 0.010263 | casein kinase 2, alpha prime polypeptide |
| | | | | (CSNK2A2) |
| NM_001896.2 | 1 | 14 | 0.004698 | casein kinase 2, alpha prime polypeptide |
| | | | | (CSNK2A2), mRNA |
| NM_001896.2 | 0 | 9 | 0.016771 | casein kinase 2, alpha prime polypeptide |
| | | | | (CSNK2A2) |
| NM_001952.2 | 0 | 10 | 0.009224 | E2F transcription factor 6 (E2F6) |
| NM_001997.2 | 1 | 12 | 0.017848 | Finkel-Biskis-Reilly murine sarcoma virus (FBR- |
| | | | | MuSV) ubiquitously expressed (fox derived); |
| | | | | ribosomal protein S30 (FAU) |
| NM_002129.2 | 0 | 9 | 0.016771 | high-mobility group box 2 (HMGB2), mRNA |
| NM_002159.2 | 0 | 9 | 0.016771 | histatin 1, mRNA, complete cds. |
| NM_002412.1 | 0 | 11 | 0.004855 | O-6-methylguanine-DNA methyltransferase (MGMT) |
| NM_002788.1 | 0 | 9 | 0.016771 | proteasome (prosome, macropain) subunit, alpha |
| | | | | type, 3 (PSMA3) |
| NM_003092.3 | 2 | 16 | 0.005343 | small nuclear ribonucleoprotein polypeptide B″ |
| | | | | (SNRPB2), transcript variant 1, mRNA |
| NM_003321.3 | 0 | 9 | 0.016771 | Tu translation elongation factor, mitochondrial |
| | | | | (TUFM), mRNA |
| NM_003516.2 | 0 | 10 | 0.009224 | histone 2, H2aa (HIST2H2AA), mRNA |
| NM_003600.1 | 0 | 11 | 0.004855 | serine/threonine kinase 6 (STK6) |
| NM_003910.2 | 0 | 9 | 0.016771 | maternal G10 transcript (G10), mRNA |
| NM_003915.2 | 0 | 9 | 0.016771 | copine I (CPNE1), transcript variant 3, mRNA |
| NM_004114.2 | 0 | 14 | 0.0005 | fibroblast growth factor 13 (FGF13), transcript |
| | | | | variant 1A, mRNA |
| NM_004214.3 | 1 | 16 | 0.000862 | fibroblast growth factor (acidic) intracellular binding |
| | | | | protein (FIBP) |
| NM_004217.1 | 1 | 15 | 0.002128 | aurora kinase B (AURKB), |
| NM_004596.1 | 0 | 16 | 7.14E−05 | small nuclear ribonucleoprotein polypeptide A |
| | | | | (SNRPA) |
| NM_004635.2 | 0 | 10 | 0.009224 | mitogen-activated protein kinase-activated protein |
| | | | | kinase 3 (MAPKAPK3) |
| NM_004645.1 | 0 | 9 | 0.016771 | coilin (COIL) |
| NM_004656.2 | 1 | 14 | 0.004698 | BRCA1 associated protein-1 (ubiquitin carboxy- |
| | | | | terminal hydrolase) (BAP1), mRNA |
| NM_004718.2 | 0 | 9 | 0.016771 | cytochrome c oxidase subunit VIIa polypeptide 2 |
| | | | | like (COX7A2L), nuclear gene encoding |
| | | | | mitochondrial protein, mRNA |
| NM_004873.1 | 0 | 9 | 0.016771 | BCL2-associated athanogene 5 (BAG5) |
| NM_004906.3 | 0 | 10 | 0.009224 | Wilms tumor 1 associated protein (WTAP), |
| | | | | transcript variant 1, mRNA |
| NM_004966.2 | 2 | 15 | 0.011617 | heterogeneous nuclear ribonucleoprotein F |
| | | | | (HNRPF), mRNA |
| NM_005011.2 | 0 | 10 | 0.009224 | nuclear respiratory factor 1 (NRF1), mRNA |
| NM_005240.1 | 0 | 10 | 0.009224 | ets variant gene 3 (ETV3), mRNA |
| NM_006205.1 | 0 | 9 | 0.016771 | phosphodiesterase 6H, cGMP-specific, cone, |
| | | | | gamma (PDE6H), mRNA |
| NM_006251.4 | 0 | 9 | 0.016771 | protein kinase, AMP-activated, alpha 1 catalytic |
| | | | | subunit (PRKAA1), transcript variant 1, mRNA |
| NM_006298.2 | 0 | 9 | 0.016771 | zinc finger protein 192 (ZNF192), mRNA |
| NM_006299.2 | 9 | 11 | 0.016771 | zinc finger protein 193 (ZNF193), mRNA |
| NM_006337.3 | 0 | 10 | 0.009224 | microspherule protein 1 (MCRS1), mRNA |
| NM_006428.3 | 0 | 9 | 0.016771 | mitochondrial ribosomal protein L28 (MRPL28), |
| | | | | nuclear gene encoding mitochondrial protein, mRNA |
| NM_006701.2 | 0 | 12 | 0.002427 | thioredoxin-like 4A (TXNL4A), mRNA |
| NM_006775.1 | 9 | 11 | 0.016771 | quaking homolog, KH domain RNA binding (mouse) |
| | | | | (QKI), transcript variant 1, mRNA |
| NM_006869.1 | 0 | 9 | 0.016771 | centaurin, alpha 1 (CENTA1), mRNA |
| NM_006931.1 | 0 | 9 | 0.016771 | solute carrier family 2 (facilitated glucose |
| | | | | transporter), member 3 (SLC2A3), mRNA |
| NM_007054.1 | 0 | 13 | 0.001142 | kinesin family member 3A (KIF3A) |
| NM_007285.5 | 0 | 12 | 0.002427 | GABA(A) receptor-associated protein-like 2 |
| | | | | (GABARAPL2) |
| NM_012153.1 | 0 | 10 | 0.009224 | ets homologous factor (EHF) |
| NM_012179.2 | 9 | 10 | 0.009224 | F-box only protein 7 (FBXO7) |
| NM_012279.1 | 0 | 9 | 0.016771 | double-stranded RNA-binding zinc finger protein |
| | | | | JAZ (JAZ) |
| NM_013257.2 | 0 | 9 | 0.016771 | serum/glucocorticoid regulated kinase-like (SGKL) |
| NM_013375.2 | 0 | 9 | 0.016771 | activator of basal transcription 1 (ABT1), mRNA |
| NM_013401.2 | 0 | 9 | 0.016771 | RAB3A interacting protein (rabin3)-like 1 (RAB3IL1), |
| | | | | mRNA |
| NM_014047.1 | 1 | 14 | 0.004698 | HSPC023 protein (HSPC023), mRNA |
| NM_014466.2 | 9 | 10 | 0.009224 | tektin 2 (testicular) (TEKT2), mRNA |
| NM_014763.2 | 0 | 10 | 0.009224 | mitochondrial ribosomal protein L19 (MRPL19), |
| | | | | nuclear gene encoding mitochondrial protein, mRNA |
| NM_014878.2 | 0 | 13 | 0.001142 | KIAA0117 protein (KIAA0117), mRNA |
| NM_015014.1 | 0 | 12 | 0.002427 | KIAA0117 protein (KIAA0117) |
| NM_015414.2 | 0 | 9 | 0.016771 | ribosomal protein L36 (RPL36), transcript variant 2 |
| NM_015464.1 | 2 | 16 | 0.005343 | sclerostin domain containing 1 (SOSTDC1), mRNA |
| NM_015488.1 | 1 | 12 | 0.017848 | myofibrillogenesis regulator 1 (MR-1) |
| NM_015640.1 | 0 | 14 | 0.0005 | PAI-1 mRNA-binding protein (PAI-RBP1) |
| NM_015698.2 | 9 | 11 | 0.016771 | T54 protein (T54) |
| NM_015933.1 | 0 | 14 | 0.0005 | hypothetical protein HSPC016 (HSPC016) |
| NM_015971.2 | 1 | 12 | 0.017848 | mitochondrial ribosomal protein S7 (MRPS7), |
| | | | | nuclear gene encoding mitochondrial protein, mRNA |
| NM_015987.2 | 1 | 14 | 0.004698 | heme binding protein 1 (HEBP1) |
| NM_016073.2 | 0 | 9 | 0.016771 | hepatoma-derived growth factor, related protein 3 |
| | | | | (HDGFRP3), mRNA |
| NM_016321.1 | 0 | 9 | 0.016771 | Rhesus blood group, C glycoprotein (RHCG), |
| | | | | mRNA |
| NM_016355.3 | 0 | 9 | 0.016771 | DEAD (Asp-Glu-Ala-Asp) box polypeptide 47 |
| | | | | (DDX47), transcript variant 1, mRNA |
| NM_016483.3 | 2 | 15 | 0.011617 | PHD finger protein 7 (PHF7) |
| NM_016487.1 | 0 | 10 | 0.009224 | HSPC230 gene (HSPC230) |
| NM_016505.2 | 1 | 15 | 0.002128 | putative S1 RNA binding domain protein (PS1D), |
| | | | | mRNA |
| NM_016940.1 | 0 | 12 | 0.002427 | chromosome 21 open reading frame 6 (C21orf6), |
| | | | | mRNA |
| NM_017503.2 | 0 | 9 | 0.016771 | surfeit 2 (SURF2), mRNA |
| NM_017588.1 | 0 | 10 | 0.009224 | WD repeat domain 5 (WDR5), transcript variant 1 |
| NM_017692.1 | 0 | 10 | 0.009224 | aprataxin (APTX) |
| NM_017838.2 | 0 | 11 | 0.004855 | nucleolar protein family A, member 2 (H/ACA small |
| | | | | nucleolar RNPs) (NOLA2) |
| NM_017846.3 | 0 | 11 | 0.004855 | tRNA selenocysteine associated protein (SECP43), |
| | | | | mRNA |
| NM_017866.3 | 0 | 9 | 0.016771 | hypothetical protein FLJ20533 (FLJ20533), mRNA |
| NM_017868.2 | 0 | 9 | 0.016771 | tetratricopeptide repeat domain 12 (TTC12) |
| NM_018032.2 | 1 | 15 | 0.002128 | LUC7-like (S. cerevisiae) (LUC7L) |
| NM_018047.1 | 0 | 15 | 0.0002 | RNA binding motif protein 22 (RBM22), mRNA |
| NM_018454.4 | 0 | 11 | 0.004855 | nucleolar and spindle associated protein 1 |
| | | | | (NUSAP1) |
| NM_018683.2 | 9 | 11 | 0.016771 | zinc finger protein 313 (ZNF313) |
| NM_019021.1 | 1 | 13 | 0.009495 | hypothetical protein FLJ20010 (FLJ20010), mRNA |
| NM_019069.3 | 0 | 10 | 0.009224 | WD repeat domain 5B (WDR5B), mRNA |
| NM_019099.1 | 0 | 9 | 0.016771 | hypothetical protein LOC55924 (LOC55924) |
| NM_020239.2 | 1 | 14 | 0.004698 | small protein effector 1 of Cdc42 (SPEC1) |
| NM_020317.2 | 9 | 10 | 0.009224 | hypothetical protein dJ465N24.2.1 |
| NM_020530.2 | 1 | 14 | 0.004698 | oncostatin M (OSM) |
| NM_020661.1 | 0 | 9 | 0.016771 | activation-induced cytidine deaminase (AICDA), |
| | | | | mRNA |
| NM_021218.1 | 2 | 16 | 0.005343 | chromosome 9 open reading frame 80 (C9orf80), |
| | | | | mRNA |
| NM_021627.2 | 0 | 9 | 0.016771 | SUMO1/sentrin/SMT3 specific protease 2 (SENP2), |
| | | | | mRNA |
| NM_021925.1 | 9 | 11 | 0.016771 | hypothetical protein FLJ21820 (FLJ21820), mRNA |
| NM_022100.1 | 0 | 13 | 0.001142 | mitochondrial ribosomal protein S14 (MRPS14), |
| | | | | nuclear gene encoding mitochondrial protein, mRNA |
| NM_022107.1 | 0 | 9 | 0.016771 | G-protein signalling modulator 3 (AGS3-like, C. elegans) |
| | | | | (GPSM3), mRNA |
| NM_022787.2 | 1 | 16 | 0.000862 | nicotinamide nucleotide adenylyltransferase 1 |
| | | | | (NMNAT1), mRNA |
| NM_022839.2 | 0 | 14 | 0.0005 | mitochondrial ribosomal protein S11 (MRPS11), |
| | | | | nuclear gene encoding mitochondrial protein, |
| | | | | transcript variant 1, mRNA |
| NM_024313.1 | 0 | 9 | 0.016771 | hypothetical protein MGC3731 (MGC3731) |
| NM_024749.1 | 9 | 10 | 0.009224 | hypothetical protein FLJ12505 |
| NM_025061.2 | 0 | 9 | 0.016771 | hypothetical protein FLJ23420 (FLJ23420) |
| NM_031452.1 | 0 | 9 | 0.016771 | hypothetical protein MGC2560 (MGC2560) |
| NM_031465.2 | 0 | 10 | 0.009224 | hypothetical protein, mRNA |
| NM_031473.1 | 9 | 10 | 0.009224 | carnitine deficiency-associated gene expressed in |
| | | | | ventricle 1 (CDV-1), |
| NM_031910.2 | 1 | 16 | 0.000862 | C1q and tumor necrosis factor related protein 6 |
| | | | | (C1QTNF6) |
| NM_031991.1 | 0 | 11 | 0.004855 | polypyrimidine tract binding protein 1 (PTBP1), |
| | | | | transcript variant 3 |
| NM_032111.2 | 0 | 10 | 0.009224 | mitochondrial ribosomal protein L14 (MRPL14), |
| | | | | nuclear gene encoding mitochondrial protein, mRNA |
| NM_032284.1 | 0 | 9 | 0.016771 | hypothetical protein FLJ14936 (FLJ14936) |
| NM_032338.2 | 0 | 9 | 0.016771 | hypothetical protein, mRNA |
| NM_032345.1 | 0 | 10 | 0.009224 | PYM protein (PYM), mRNA |
| NM_032359.1 | 0 | 12 | 0.002427 | hypothetical protein MGC4308 |
| NM_032459.1 | 0 | 9 | 0.016771 | embryonal Fyn-associated substrate (EFS), |
| | | | | transcript variant 2, mRNA |
| NM_032848.1 | 0 | 11 | 0.004855 | hypothetical protein FLJ14827 (FLJ14827), mRNA |
| NM_032855.1 | 9 | 11 | 0.016771 | hematopoietic SH2 protein (HSH2) |
| NM_033048.1 | 0 | 9 | 0.016771 | CPX chromosome region, candidate 1 (CPXCR1) |
| NM_033177.2 | 1 | 12 | 0.017848 | HLA-B associated transcript 4 (BAT4), mRNA |
| NM_033345.1 | 0 | 10 | 0.009224 | regulator of G-protein signalling 8 (RGS8) |
| NM_033416.1 | 0 | 10 | 0.009224 | IMP4, U3 small nucleolar ribonucleoprotein, |
| | | | | homolog (yeast) (IMP4), mRNA |
| NM_052844.1 | 9 | 11 | 0.016771 | hypothetical protein MGC20486 |
| NM_052848.1 | 9 | 10 | 0.009224 | hypothetical protein, mRNA |
| NM_054016.1 | 1 | 12 | 0.017848 | FUS interacting protein (serine-arginine rich) 1 |
| | | | | (FUSIP1), transcript variant 2, mRNA |
| NM_138451.1 | 0 | 9 | 0.016771 | IQ motif containing D (IQCD), mRNA |
| NM_138612.1 | 9 | 11 | 0.016771 | hyaluronan synthase 3 (HAS3), transcript variant 2, |
| | | | | mRNA |
| NM_138775.1 | 0 | 11 | 0.004855 | hypothetical protein BC015183 (LOC91801), mRNA |
| NM_138777.1 | 0 | 9 | 0.016771 | mitochondrial ribosome recycling factor (MRRF) |
| NM_138959.1 | 1 | 13 | 0.009495 | vang-like 1 |
| NM_144595.1 | 0 | 9 | 0.016771 | hypothetical protein FLJ30046 |
| NM_144679.1 | 0 | 9 | 0.016771 | hypothetical protein FLJ31528 (FLJ31528), mRNA |
| NM_144714.1 | 0 | 10 | 0.009224 | hypothetical protein MGC27069 |
| NM_144769.1 | 0 | 9 | 0.016771 | forkhead box I1 (FOXI1), transcript variant 2, mRNA |
| NM_144971.1 | 0 | 15 | 0.0002 | hypothetical protein MGC26641 (MGC26641) |
| NM_144982.1 | 2 | 18 | 0.000701 | hypothetical protein MGC23401 (MGC23401) |
| NM_145204.1 | 9 | 11 | 0.016771 | NEDD8-specific protease 1 (SENP8) |
| NM_145691.3 | 0 | 9 | 0.016771 | ATP synthase mitochondrial F1 complex assembly |
| | | | | factor 2 (ATPAF2), nuclear gene encoding |
| | | | | mitochondrial protein, mRNA |
| NM_145802.1 | 4 | 19 | 0.005482 | septin 6 (SEPT6) |
| NM_145810.1 | 2 | 15 | 0.011617 | cell division cycle associated 7 (CDCA7), transcript |
| | | | | variant 2 |
| NM_152255.1 | 0 | 9 | 0.016771 | proteasome (prosome, macropain) subunit, alpha |
| | | | | type, 7 (PSMA7), transcript variant 2 |
| NM_152324.1 | 0 | 9 | 0.016771 | hypothetical protein, mRNA |
| NM_152376.2 | 0 | 9 | 0.016771 | UBX domain containing 3 (UBXD3), mRNA |
| NM_152397.1 | 0 | 10 | 0.009224 | IQ motif containing F1 (IQCF1), mRNA |
| NM_152474.2 | 0 | 14 | 0.0005 | chromosome 19 open reading frame 18 (C19orf18), |
| | | | | mRNA |
| NM_152638.2 | 0 | 11 | 0.004855 | chromosome 12 open reading frame 12 (C12orf12), |
| | | | | mRNA |
| NM_152769.1 | 1 | 16 | 0.000862 | chromosome 19 open reading frame 26 (C19orf26), |
| | | | | mRNA |
| NM_153207.2 | 0 | 10 | 0.009224 | AE binding protein 2 (AEBP2) |
| NM_153332.2 | 0 | 10 | 0.009224 | 3′ exoribonuclease (3′HEXO), mRNA |
| NM_173519.1 | 9 | 11 | 0.016771 | hypothetical protein, mRNA |
| NM_175923.2 | 2 | 17 | 0.002135 | hypothetical protein MGC42630 |
| NM_178496.2 | 9 | 11 | 0.016771 | similar to BcDNA: GH11415 gene product |
| | | | | (LOC151963), mRNA |
| NM_182692.1 | 0 | 11 | 0.004855 | SFRS protein kinase 2 (SRPK2), transcript variant |
| | | | | 1, mRNA |
| NM_199334.2 | 0 | 10 | 0.009224 | thyroid hormone receptor, alpha (erythroblastic |
| | | | | leukemia viral (v-erb-a) oncogene homolog, avian) |
| | | | | (THRA), transcript variant 1, mRNA |
| NM_199415.1 | 9 | 10 | 0.009224 | U-box domain containing 5 (UBOX5), transcript |
| | | | | variant 2, mRNA |
| NM_203454.1 | 0 | 9 | 0.016771 | hypothetical protein, mRNA |
| NM_206834.1 | 0 | 9 | 0.016771 | chromosome 6 open reading frame 201 (C6orf201), |
| | | | | mRNA |

TABLE 11
Table 11 is a list of proteins that were bound by an antibody from SLE patient
sera but not ANCA patients.

| Genbank ID | | | | |
|---|---|---|---|---|
| number of | | | | |
| nucleic acid | | | | |
| coding for | SLE | ANCA | | |
| the protein | Count | Count | p-value | Name or description |
| AB065619.1 | 3 | 18 | 0.000122 | gene for seven transmembrane helix receptor, |
| AB065812.1 | 1 | 12 | 0.011239 | gene for seven transmembrane helix receptor, |
| | | | | complete cds, isolate: CBRC7TM_375 |
| BC000166.2 | 3 | 12 | 0.003956 | cDNA clone IMAGE: 2901054 |
| BC000381.2 | 0 | 10 | 0.000218 | TBP-like 1, mRNA |
| BC000877.1 | 2 | 10 | 0.006907 | vasopressin-induced transcript |
| BC000934.2 | 0 | 6 | 0.010098 | eukaryotic translation initiation factor 2, subunit 2 |
| | | | | beta, 38 kDa, mRNA |
| BC000954.1 | 6 | 17 | 0.009351 | chromobox homolog 3 (HP1 gamma homolog, |
| | | | | Drosophila), transcript variant 1 |
| BC000997.2 | 0 | 11 | 7.27E−05 | splicing factor, arginine/serine-rich 7, 35 kDa |
| BC001280.1 | 1 | 9 | 0.015475 | serine/threonine kinase 6, transcript variant 1 |
| BC002424.1 | 0 | 8 | 0.001638 | integral membrane protein 2C, transcript variant 1, |
| | | | | mRNA |
| BC002559.1 | 2 | 12 | 0.0011 | high-glucose-regulated protein 8, clone MGC: 739 |
| | | | | IMAGE: 3139250 |
| BC002606.1 | 6 | 15 | 0.012421 | Similar to hypothetical protein, clone MGC: 2992 |
| | | | | IMAGE: 3160695 |
| BC002733.2 | 3 | 12 | 0.003956 | mRNA, complete cds. |
| BC005248.1 | 3 | 11 | 0.009351 | eukaryotic translation initiation factor 1A, Y-linked |
| BC005955.1 | 3 | 12 | 0.011239 | hypothetical protein MGC14595 |
| BC006376.1 | 0 | 7 | 0.004158 | N-myristoyltransferase 2 |
| BC006793.1 | 4 | 13 | 0.012421 | GATA binding protein 3 |
| BC007228.1 | 4 | 19 | 0.000624 | similar to Taxol resistant associated protein 3 |
| | | | | (TRAG-3) |
| BC007347.2 | 1 | 11 | 0.002868 | Unknown (protein for MGC: 1566) |
| BC007833.2 | 0 | 14 | 1.97E−05 | phosphatidylinositol-4-phosphate 5-kinase, type I, |
| | | | | alpha, mRNA |
| BC007888.1 | 11 | 19 | 0.004181 | eukaryotic translation initiation factor 2, subunit 2 |
| | | | | (beta, 38 kD) |
| BC008623.1 | 1 | 8 | 0.009828 | hypothetical protein FLJ21044 similar to Rbig1, |
| | | | | cloneMGC: 16823 IMAGE: 4177689, mRNA, |
| | | | | complete cds. |
| BC008730.2 | 0 | 7 | 0.004158 | hexokinase 1, transcript variant 1, mRNA |
| BC008741.1 | 1 | 8 | 0.009828 | LIM protein (similar to rat protein kinase C-binding |
| | | | | enigma) |
| BC009350.1 | 1 | 9 | 0.015475 | clone MGC: 14871 IMAGE: 4137621 |
| BC009623.1 | 3 | 14 | 0.000532 | Similar to nucleophosmin (nucleolar phosphoprotein |
| | | | | B23, numatrin) |
| BC009650.1 | 1 | 10 | 0.001671 | mRNA, complete cds. |
| BC009762.2 | 4 | 13 | 0.004765 | mRNA, complete cds. |
| BC009819.1 | 3 | 12 | 0.003956 | hypothetical protein FLJ23591 |
| BC010074.2 | 5 | 17 | 0.003956 | FUS interacting protein (serine/arginine-rich) 1, |
| | | | | mRNA |
| BC010356.1 | 2 | 9 | 0.015475 | gi\|14714460 hypothetical protein |
| BC010360.1 | 6 | 17 | 0.003956 | Unknown (protein for MGC: 13386) |
| BC010467.1 | 8 | 18 | 0.002868 | cDNA clone MGC: 17410 IMAGE: 4156035 |
| BC010642.1 | 2 | 10 | 0.006907 | zinc finger protein 22 (KOX 15) |
| BC010697.1 | 1 | 11 | 0.000624 | amylase, alpha 2B; pancreatic |
| BC010907.1 | 0 | 9 | 0.015475 | PAK1 interacting protein 1, mRNA |
| BC010947.1 | 0 | 11 | 7.27E−05 | signal recognition particle 19 kDa, mRNA |
| BC011379.1 | 2 | 11 | 0.002868 | DKFZP434H132 protein |
| BC011498.1 | 0 | 6 | 0.010098 | Unknown (protein for MGC: 17017) |
| BC011600.1 | 0 | 6 | 0.010098 | Similar to RD RNA-binding protein, clone |
| | | | | MGC: 2263 |
| BC011804.2 | 2 | 11 | 0.009351 | chromosome 1 open reading frame 165, mRNA |
| BC011842.2 | 1 | 11 | 0.000624 | hypothetical protein FLJ11184, mRNA |
| BC012462.1 | 0 | 6 | 0.010098 | clone MGC: 21750 IMAGE: 4537558 |
| BC012472.1 | 6 | 18 | 0.000122 | ubiquitin D, mRNA |
| BC012566.1 | 0 | 6 | 0.010098 | nucleophosmin (nucleolar phosphoprotein B23, |
| | | | | numatrin), mRNA |
| BC012865.1 | 1 | 9 | 0.015475 | retinoic acid induced 16 |
| BC012926.1 | 1 | 9 | 0.004181 | EPS8-like 3, transcript variant 1, mRNA |
| BC013051.1 | 0 | 7 | 0.004158 | LIM domain kinase 2 |
| BC013437.1 | 2 | 9 | 0.015475 | Similar to MADS box transcription enhancer factor |
| | | | | 2, polypeptide A (myocyte enhancer factor 2A) |
| BC014218.2 | 2 | 12 | 0.011239 | cDNA clone IMAGE: 3954254 |
| BC014298.1 | 2 | 11 | 0.002868 | likely ortholog of mouse C114 dsRNA-binding |
| | | | | protein |
| BC014452.1 | 4 | 16 | 0.000616 | cDNA clone IMAGE: 4903661 |
| BC015008.1 | 8 | 16 | 0.011239 | hydroxyacylglutathione hydrolase-like, mRNA |
| BC016609.1 | 0 | 8 | 0.009828 | cytidine monophosphate N-acetylneuraminic acid |
| | | | | synthetase, mRNA |
| BC016768.1 | 2 | 11 | 0.002868 | nucleophosmin (nucleolar phosphoprotein B23, |
| | | | | numatrin), mRNA |
| BC016778.1 | 2 | 10 | 0.006907 | HIV-1 rev binding protein 2, mRNA |
| BC016842.1 | 2 | 11 | 0.002868 | family with sequence similarity 61, member A, |
| | | | | mRNA |
| BC017020.1 | 4 | 15 | 0.012421 | single-stranded DNA binding protein 2 |
| BC017114.1 | 6 | 18 | 0.0011 | hypothetical protein FLJ22833, |
| BC017163.1 | 2 | 13 | 0.004765 | CGI-74 protein, mRNA (cDNA clone MGC: 8819 |
| | | | | IMAGE: 3920377) |
| BC018823.2 | 5 | 16 | 0.000616 | splicing factor, arginine/serine-rich |
| BC020647.1 | 0 | 7 | 0.004158 | HSPC128 protein, mRNA |
| BC021930.1 | 4 | 15 | 0.005193 | Unknown (protein for MGC: 32072) |
| BC021983.1 | 0 | 8 | 0.001638 | nucleophosmin (nucleolar phosphoprotein B23, |
| | | | | numatrin), transcript variant 1, mRNA |
| BC022077.1 | 2 | 13 | 0.000386 | hypothetical protein MGC33338 |
| BC022231.1 | 1 | 8 | 0.009828 | Ets2 repressor factor, mRNA |
| BC022325.1 | 0 | 17 | 1.28E−08 | hypothetical protein FLJ12729 |
| BC022361.1 | 0 | 6 | 0.010098 | chromosome 14 open reading frame 111, |
| BC025996.2 | 4 | 16 | 0.001821 | cDNA clone MGC: 26787 IMAGE: 4838986 |
| BC027178.1 | 8 | 18 | 0.015475 | formin binding protein 3, mRNA |
| BC027607.1 | 6 | 15 | 0.012421 | clone MGC: 26892 IMAGE: 4828241 |
| BC028301.1 | 0 | 9 | 0.000614 | similar to LOC147447 |
| BC028396.1 | 0 | 6 | 0.010098 | polyhomeotic-like 2 (Drosophila) |
| BC028425.1 | 0 | 6 | 0.010098 | KIAA0027 protein |
| BC029046.1 | 3 | 12 | 0.003956 | H1 histone family, member 0, mRNA |
| BC029427.1 | 2 | 12 | 0.0011 | hypothetical protein LOC374969 |
| BC030219.1 | 3 | 13 | 0.012421 | RAD51-like 1 (S. cerevisiae) |
| BC030711.2 | 2 | 9 | 0.015475 | chromosome 2 open reading frame 13 |
| BC031010.1 | 0 | 6 | 0.010098 | SET and MYND domain containing 3, mRNA |
| BC031281.1 | 3 | 13 | 0.004765 | tetratricopeptide repeat domain 16, mRNA |
| BC032334.1 | 7 | 16 | 0.004765 | putative homeodomain transcription factor 2, |
| | | | | mRNA, complete cds. |
| BC032449.1 | 0 | 8 | 0.001638 | paralemmin, mRNA |
| BC032852.2 | 10 | 18 | 0.006907 | melanoma antigen family B, 4, mRNA |
| BC033088.1 | 0 | 6 | 0.010098 | lamin A/C, mRNA |
| BC033159.1 | 10 | 19 | 0.009828 | DnaJ (Hsp40) homolog, subfamily C, member 8, |
| | | | | mRNA |
| BC033629.1 | 1 | 8 | 0.009828 | chromosome 20 open reading frame 77, mRNA |
| BC033856.1 | 3 | 15 | 0.000164 | Similar to RIKEN cDNA 3110040D16 gene, |
| | | | | cloneMGC: 45395 IMAGE: 5123380, mRNA, |
| | | | | complete cds. |
| BC038105.2 | 4 | 16 | 0.000616 | membrane protein, palmitoylated 7 (MAGUK p55 |
| | | | | subfamily member 7) |
| BC038808.1 | 11 | 20 | 0.001638 | apolipoprotein B mRNA editing enzyme, catalytic |
| | | | | polypeptide-like 3F, transcript variant 1, mRNA |
| BC042625.1 | 0 | 9 | 0.000614 | LUC7-like 2 (S. cerevisiae), mRNA |
| BC043564.1 | 13 | 20 | 0.010098 | potassium voltage-gated channel, shaker-related |
| | | | | subfamily, member 2, mRNA |
| BC052806.1 | 4 | 14 | 0.001821 | cDNA clone MGC: 61802 |
| BC053343.1 | 1 | 11 | 0.009351 | karyopherin alpha 2 (RAG cohort 1, importin alpha |
| | | | | 1), mRNA |
| BC053557.1 | 6 | 15 | 0.005193 | cDNA clone MGC: 61706 IMAGE: 6162269 |
| BC053866.1 | 2 | 11 | 0.009351 | endothelin 3, transcript variant 2 |
| BC054034.1 | 0 | 7 | 0.004158 | U11/U12 snRNP 35K, transcript variant 2 |
| BC055314.1 | 1 | 11 | 0.002868 | C2f protein |
| BC056508.1 | 1 | 12 | 0.0011 | variable charge, Y-linked 1B |
| BC058912.1 | 3 | 12 | 0.011239 | butyrate-induced transcript 1, mRNA |
| BC060758.1 | 0 | 12 | 0.003956 | complete cds. |
| BC063275.1 | 4 | 16 | 0.004765 | eukaryotic translation initiation factor 2C, 1, mRNA |
| BC065525.1 | 0 | 6 | 0.010098 | adducin 2 (beta), mRNA |
| BC067446.1 | 1 | 8 | 0.009828 | disabled homolog 1 (Drosophila), mRNA |
| CTL2110 | 0 | 11 | 7.27E−05 | DNA TOPOISIMERASE(Scl-70) |
| CTL2112 | 3 | 18 | 8.32E−06 | ssDNA |
| CTL2121 | 1 | 8 | 0.009828 | Ro-52 |
| CTL2132 | 1 | 11 | 0.000624 | myeloperoxidase |
| CTL2136 | 1 | 11 | 0.000624 | U1-snRNP 68 PROTEIN |
| CTL2138 | 0 | 14 | 1.67E−06 | RNP COMPLEX |
| CTL2139 | 1 | 10 | 0.001671 | UNFRAC. WHOLE HISTONE |
| CTL2142 | 1 | 14 | 1.97E−05 | ssDNA |
| CTL2143 | 12 | 19 | 0.009828 | CENTROMERE PRO B |
| CTL2145 | 0 | 12 | 2.25E−05 | RIBOSOMAL RNA |
| CTL2147 | 0 | 7 | 0.004158 | dsDNA |
| CTL2150 | 6 | 16 | 0.004765 | SMITH ANTIGEN |
| CTL2151 | 2 | 12 | 0.0011 | Ro-52 |
| NM_000723.3 | 1 | 10 | 0.001671 | calcium channel, voltage-dependent, beta 1 subunit |
| | | | | (CACNB1), transcript variant 1, mRNA |
| NM_000975.2 | 1 | 10 | 0.006907 | ribosomal protein L11 (RPL11), mRNA |
| NM_000979.2 | 11 | 19 | 0.009828 | ribosomal protein L18 (RPL18), mRNA |
| NM_000984.2 | 1 | 8 | 0.009828 | ribosomal protein L23a (RPL23A) |
| NM_000989.2 | 0 | 7 | 0.004158 | ribosomal protein L30 (RPL30), mRNA |
| NM_000997.2 | 1 | 12 | 0.011239 | ribosomal protein L37 (RPL37) |
| NM_000999.2 | 0 | 6 | 0.010098 | ribosomal protein L38 (RPL38) |
| NM_001014.2 | 1 | 14 | 1.97E−05 | ribosomal protein S10 (RPS10) |
| NM_001015.2 | 0 | 7 | 0.004158 | ribosomal protein S11 (RPS11) |
| NM_001020.2 | 5 | 16 | 0.004765 | ribosomal protein S16 (RPS16) |
| NM_001022.3 | 2 | 11 | 0.002868 | ribosomal protein S19 (RPS19), mRNA |
| NM_001023.2 | 0 | 6 | 0.010098 | ribosomal protein S20 (RPS20), mRNA |
| NM_001106.2 | 4 | 12 | 0.011239 | activin A receptor, type IIB (ACVR2B), |
| NM_001124.1 | 2 | 9 | 0.015475 | adrenomedullin (ADM), mRNA |
| NM_001616.2 | 4 | 13 | 0.004765 | activin A receptor, type II (ACVR2) |
| NM_001722.2 | 4 | 12 | 0.011239 | polymerase (RNA) III (DNA directed) polypeptide D, |
| | | | | 44 kDa (POLR3D), mRNA |
| NM_001896.1 | 2 | 10 | 0.006907 | casein kinase 2, alpha prime polypeptide |
| | | | | (CSNK2A2) |
| NM_001896.2 | 2 | 10 | 0.006907 | casein kinase 2, alpha prime polypeptide |
| | | | | (CSNK2A2), mRNA |
| NM_001997.2 | 2 | 10 | 0.006907 | Finkel-Biskis-Reilly murine sarcoma virus (FBR- |
| | | | | MuSV) ubiquitously expressed (fox derived); |
| | | | | ribosomal protein S30 (FAU) |
| NM_002129.2 | 0 | 8 | 0.001638 | high-mobility group box 2 (HMGB2), mRNA |
| NM_002446.2 | 5 | 16 | 0.000616 | mitogen-activated protein kinase kinase kinase 10 |
| | | | | (MAP3K10) |
| NM_002677.1 | 4 | 14 | 0.012822 | peripheral myelin protein 2 (PMP2) |
| NM_002734.1 | 1 | 9 | 0.004181 | protein kinase, cAMP-dependent, regulatory, type I, |
| | | | | alpha (tissue specific extinguisher 1) (PRKAR1A) |
| NM_003092.3 | 5 | 13 | 0.012421 | small nuclear ribonucleoprotein polypeptide B″ |
| | | | | (SNRPB2), transcript variant 1, mRNA |
| NM_003123.1 | 8 | 17 | 0.009351 | sialophorin (gpL115, leukosialin, CD43) (SPN) |
| NM_003295.1 | 0 | 7 | 0.004158 | tumor protein, translationally-controlled 1 (TPT1), |
| | | | | mRNA |
| NM_003390.2 | 1 | 9 | 0.004181 | WEE1 homolog (S. pombe) (WEE1) |
| NM_003516.2 | 9 | 20 | 0.001638 | histone 2, H2aa (HIST2H2AA), mRNA |
| NM_003621.1 | 0 | 8 | 0.001638 | PTPRF interacting protein, binding protein 2 (liprin |
| | | | | beta 2) (PPFIBP2), mRNA |
| NM_003688.1 | 10 | 19 | 0.004181 | calcium/calmodulin-dependent serine protein kinase |
| | | | | (MAGUK family) (CASK) |
| NM_003992.1 | 1 | 9 | 0.004181 | CDC-like kinase 3 (CLK3), transcript variant phclk3 |
| NM_004114.2 | 5 | 13 | 0.012421 | fibroblast growth factor 13 (FGF13), transcript |
| | | | | variant 1A, mRNA |
| NM_004214.3 | 0 | 14 | 1.67E−06 | fibroblast growth factor (acidic) intracellular binding |
| | | | | protein (FIBP) |
| NM_004286.2 | 2 | 9 | 0.015475 | GTP binding protein 1 (GTPBP1) |
| NM_004310.2 | 3 | 12 | 0.011239 | ras homolog gene family, member H (RHOH), |
| | | | | mRNA |
| NM_004596.1 | 0 | 13 | 6.44E−06 | small nuclear ribonucleoprotein polypeptide A |
| | | | | (SNRPA) |
| NM_004645.1 | 0 | 7 | 0.004158 | coilin (COIL) |
| NM_004966.2 | 1 | 9 | 0.004181 | heterogeneous nuclear ribonucleoprotein F |
| | | | | (HNRPF), mRNA |
| NM_005441.2 | 2 | 10 | 0.006907 | chromatin assembly factor 1, subunit B (p60) |
| | | | | (CHAF1B), mRNA |
| NM_005517.2 | 0 | 7 | 0.004158 | high-mobility group nucleosomal binding domain 2 |
| | | | | (HMGN2), mRNA |
| NM_006298.2 | 7 | 18 | 0.000386 | zinc finger protein 192 (ZNF192), mRNA |
| NM_006528.2 | 1 | 8 | 0.009828 | tissue factor pathway inhibitor 2 (TFPI2), mRNA |
| NM_006681.1 | 6 | 17 | 0.009351 | neuromedin U (NMU), mRNA |
| NM_006695.2 | 2 | 9 | 0.015475 | RaP2 interacting protein 8 (RPIP8) |
| NM_006701.2 | 1 | 9 | 0.004181 | thioredoxin-like 4A (TXNL4A), mRNA |
| NM_006788.2 | 0 | 7 | 0.004158 | ralA binding protein 1 (RALBP1) |
| NM_006791.1 | 1 | 12 | 0.0011 | mortality factor 4 like 1 (MORF4L1) |
| NM_006857.1 | 0 | 8 | 0.001638 | putative nucleic acid binding protein RY-1 (RY1), |
| | | | | mRNA |
| NM_006937.2 | 0 | 6 | 0.010098 | SMT3 suppressor of mif two 3 homolog 2 (yeast) |
| | | | | (SMT3H2) |
| NM_007054.1 | 3 | 19 | 1.97E−05 | kinesin family member 3A (KIF3A) |
| NM_007173.3 | 0 | 6 | 0.010098 | protease, serine, 23 (PRSS23), mRNA |
| NM_007285.5 | 5 | 16 | 0.004765 | GABA(A) receptor-associated protein-like 2 |
| | | | | (GABARAPL2) |
| NM_012153.1 | 3 | 12 | 0.003956 | ets homologous factor (EHF) |
| NM_012279.1 | 12 | 19 | 0.009828 | double-stranded RNA-binding zinc finger protein |
| | | | | JAZ (JAZ), |
| NM_012316.2 | 0 | 6 | 0.010098 | karyopherin alpha 6 (importin alpha 7) (KPNA6) |
| NM_012321.1 | 0 | 10 | 0.001671 | U6 snRNA-associated Sm-like protein (LSM4) |
| NM_012425.2 | 1 | 9 | 0.015475 | Ras suppressor protein 1 (RSU1) |
| NM_013293.1 | 0 | 12 | 0.000216 | transformer-2 alpha (TRA2A) |
| NM_014047.1 | 6 | 16 | 0.004765 | HSPC023 protein (HSPC023), mRNA |
| NM_014765.1 | 9 | 18 | 0.015475 | translocase of outer mitochondrial membrane 20 |
| | | | | homolog (yeast) (TOMM20), mRNA |
| NM_015014.1 | 8 | 16 | 0.011239 | KIAA0117 protein (KIAA0117), mRNA |
| NM_015149.2 | 0 | 7 | 0.004158 | ral guanine nucleotide dissociation stimulator-like 1 |
| | | | | (RGL1), mRNA |
| NM_015464.1 | 1 | 11 | 0.000624 | sclerostin domain containing 1 (SOSTDC1), mRNA |
| NM_015488.1 | 6 | 17 | 0.001528 | myofibrillogenesis regulator 1 (MR-1) |
| NM_015640.1 | 3 | 13 | 0.001528 | PAI-1 mRNA-binding protein (PAI-RBP1) |
| NM_015933.1 | 2 | 13 | 0.000386 | hypothetical protein HSPC016 (HSPC016) |
| NM_015971.2 | 0 | 6 | 0.010098 | mitochondrial ribosomal protein S7 (MRPS7), |
| | | | | nuclear gene encoding mitochondrial protein, mRNA |
| NM_015987.2 | 4 | 18 | 8.32E−06 | heme binding protein 1 (HEBP1) |
| NM_016000.2 | 0 | 6 | 0.010098 | tRNA nucleotidyl transferase, CCA-adding, 1 |
| | | | | (TRNT1), mRNA |
| NM_016303.1 | 5 | 14 | 0.012822 | pp21 homolog (LOC51186) |
| NM_016321.1 | 5 | 13 | 0.012421 | Rhesus blood group, C glycoprotein (RHCG), |
| | | | | mRNA |
| NM_016355.3 | 0 | 13 | 6.44E−06 | DEAD (Asp-Glu-Ala-Asp) box polypeptide 47 |
| | | | | (DDX47), transcript variant 1, mRNA |
| NM_016483.3 | 2 | 15 | 0.000164 | PHD finger protein 7 (PHF7) |
| NM_016505.2 | 1 | 13 | 6.85E−05 | putative S1 RNA binding domain protein (PS1D), |
| | | | | mRNA |
| NM_016520.1 | 1 | 8 | 0.009828 | chromosome 9 open reading frame 78 (C9orf78), |
| | | | | mRNA |
| NM_016606.2 | 9 | 20 | 0.001638 | chromosome 5 open reading frame 19 (C5orf19), |
| | | | | mRNA |
| NM_016940.1 | 4 | 18 | 0.000386 | chromosome 21 open reading frame 6 (C21orf6), |
| | | | | mRNA |
| NM_017588.1 | 2 | 14 | 0.000122 | WD repeat domain 5 (WDR5), transcript variant 1 |
| NM_017692.1 | 0 | 7 | 0.004158 | aprataxin (APTX) |
| NM_017838.2 | 1 | 14 | 1.97E−05 | nucleolar protein family A, member 2 (H/ACA small |
| | | | | nucleolar RNPs) (NOLA2) |
| NM_017846.3 | 0 | 8 | 0.001638 | tRNA selenocysteine associated protein (SECP43), |
| | | | | mRNA |
| NM_018032.2 | 2 | 13 | 0.000386 | LUC7-like (S. cerevisiae) (LUC7L) |
| NM_018105.1 | 3 | 11 | 0.009351 | THAP domain containing, apoptosis associated |
| | | | | protein 1 (THAP1) |
| NM_018107.2 | 4 | 14 | 0.001821 | RNA-binding region (RNP1, RRM) containing 4 |
| | | | | (RNPC4) |
| NM_018710.1 | 13 | 20 | 0.010098 | hypothetical protein DKFZp762O076 |
| | | | | (DKFZp762O076), mRNA |
| NM_019099.1 | 2 | 10 | 0.006907 | hypothetical protein LOC55924 |
| NM_020239.2 | 1 | 11 | 0.000624 | small protein effector 1 of Cdc42 (SPEC1) |
| NM_020530.2 | 7 | 17 | 0.009351 | oncostatin M (OSM) |
| NM_020648.3 | 14 | 20 | 0.010098 | twisted gastrulation homolog 1 (Drosophila) |
| | | | | (TWSG1), mRNA |
| NM_020661.1 | 0 | 9 | 0.000614 | activation-induced cytidine deaminase (AICDA), |
| | | | | mRNA |
| NM_021104.1 | 0 | 6 | 0.010098 | ribosomal protein L41 (RPL41), mRNA |
| NM_022142.3 | 1 | 12 | 0.011239 | epididymal sperm binding protein 1 (ELSPBP1), |
| | | | | mRNA |
| NM_022839.2 | 3 | 11 | 0.009351 | mitochondrial ribosomal protein S11 (MRPS11), |
| | | | | nuclear gene encoding mitochondrial protein, |
| | | | | transcript variant 1, mRNA |
| NM_024068.1 | 1 | 10 | 0.001671 | hypothetical protein MGC2731 (MGC2731) |
| NM_024482.1 | 0 | 7 | 0.004158 | glucocorticoid modulatory element binding protein 1 |
| | | | | (GMEB1), transcript variant 2, mRNA |
| NM_024625.3 | 1 | 14 | 0.000532 | zinc finger CCCH type, antiviral 1 (ZC3HAV1), |
| | | | | transcript variant 2, mRNA |
| NM_031412.1 | 0 | 10 | 0.000218 | GABA(A) receptor-associated protein like 1 |
| | | | | (GABARAPL1) |
| NM_031452.1 | 5 | 15 | 0.012421 | hypothetical protein MGC2560 |
| NM_031910.2 | 6 | 17 | 0.000532 | C1q and tumor necrosis factor related protein 6 |
| | | | | (C1QTNF6) |
| NM_031991.1 | 9 | 20 | 0.000614 | polypyrimidine tract binding protein 1 (PTBP1), |
| | | | | transcript variant 3 |
| NM_032042.2 | 6 | 15 | 0.005193 | hypothetical protein DKFZp564D172 |
| NM_032338.2 | 1 | 8 | 0.009828 | hypothetical protein, mRNA |
| NM_032345.1 | 4 | 16 | 0.00018 | PYM protein (PYM), mRNA |
| NM_032883.1 | 1 | 10 | 0.001671 | chromosome 20 open reading frame 100 |
| | | | | (C20orf100), mRNA |
| NM_033048.1 | 3 | 13 | 0.012421 | CPX chromosome region, candidate 1 (CPXCR1) |
| NM_033177.2 | 2 | 12 | 0.011239 | HLA-B associated transcript 4 (BAT4), mRNA |
| NM_033642.1 | 2 | 9 | 0.015475 | fibroblast growth factor 13 (FGF13), transcript |
| | | | | variant 1B, mRNA |
| NM_054016.1 | 1 | 10 | 0.001671 | FUS interacting protein (serine-arginine rich) 1 |
| | | | | (FUSIP1), transcript variant 2, mRNA |
| NM_058199.1 | 2 | 12 | 0.003956 | olfactomedin 1 (OLFM1), transcript variant 3 |
| NM_138775.1 | 2 | 15 | 0.000164 | hypothetical protein BC015183 (LOC91801), mRNA |
| NM_144590.1 | 0 | 9 | 0.004181 | ankyrin repeat domain 22 (ANKRD22), mRNA |
| NM_144608.1 | 0 | 9 | 0.000614 | hypothetical protein (FLJ32384), mRNA |
| NM_144659.1 | 0 | 8 | 0.001638 | t-complex 10 (mouse)-like (TCP10L) |
| NM_144971.1 | 3 | 11 | 0.009351 | hypothetical protein MGC26641 |
| NM_144982.1 | 3 | 13 | 0.001528 | hypothetical protein MGC23401 |
| NM_145010.1 | 9 | 20 | 0.000218 | hypothetical protein MGC26778 |
| NM_152362.1 | 0 | 8 | 0.001638 | hypothetical protein, mRNA |
| NM_152397.1 | 0 | 9 | 0.000614 | IQ motif containing F1 (IQCF1), mRNA |
| NM_152638.2 | 1 | 8 | 0.009828 | chromosome 12 open reading frame 12 (C12orf12), |
| | | | | mRNA |
| NM_152769.1 | 1 | 8 | 0.009828 | chromosome 19 open reading frame 26 (C19orf26), |
| | | | | mRNA |
| NM_153207.2 | 10 | 18 | 0.006907 | AE binding protein 2 (AEBP2) |
| NM_153332.2 | 4 | 16 | 0.000616 | 3′ exoribonuclease (3′HEXO), mRNA |
| NM_170676.2 | 2 | 11 | 0.002868 | Meis1, myeloid ecotropic viral integration site 1 |
| | | | | homolog 2 (mouse) (MEIS2), transcript variant d, |
| | | | | mRNA |
| NM_173545.1 | 1 | 8 | 0.009828 | chromosome 2 open reading frame 13 (C2orf13), |
| | | | | mRNA |
| NM_177924.1 | 1 | 8 | 0.009828 | N-acylsphingosine amidohydrolase (acid |
| | | | | ceramidase) 1 (ASAH1), transcript variant 1, mRNA |
| NM_178861.3 | 4 | 17 | 0.001528 | zinc finger protein 183-like 1 (ZNF183L1), mRNA |
| NM_182623.1 | 12 | 20 | 0.010098 | hypothetical protein FLJ36766 (FLJ36766), mRNA |
| NM_198395.1 | 8 | 18 | 0.0011 | Ras-GTPase-activating protein SH3-domain-binding |
| | | | | protein (G3BP), transcript variant 2 |
| NM_199334.2 | 0 | 6 | 0.010098 | thyroid hormone receptor, alpha (erythroblastic |
| | | | | leukemia viral (v-erb-a) oncogene homolog, avian) |
| | | | | (THRA), transcript variant 1, mRNA |
| NM_203350.1 | 1 | 9 | 0.004181 | zinc finger protein 265 (ZNF265), transcript variant |
| | | | | 1, mRNA |

TABLE 12
Table 12 is a list of proteins that were bound by an antibody from ANCA
patient sera but not SLE patients.

| Genbank ID | | | | |
|---|---|---|---|---|
| number of | | | | |
| nucleic acid | | | | |
| coding for | ANCA | SLE | | |
| the protein | Count | Count | p-value | Name or description |
| BC000052.1 | 8 | 1 | 0.009828 | Similar to peroxisome proliferative activated |
| | | | | receptor, alpha, clone MGC: 2237 |
| BC000103.1 | 17 | 6 | 0.0015282 | NCK adaptor protein 2 |
| BC000733.1 | 6 | 0 | 0.010098 | eukaryotic translation initiation factor 3, subunit 4 |
| | | | | (delta, 44 kD), |
| BC000979.2 | 12 | 2 | 0.0010998 | DEAD (Asp-Glu-Ala-Asp) box polypeptide 49 |
| BC001132.1 | 9 | 2 | 0.0154752 | DEAD (Asp-Glu-Ala-Asp) box polypeptide 54 |
| BC001152.1 | 12 | 0 | 0.0002158 | growth arrest-specific 7, mRNA |
| BC001286.1 | 7 | 0 | 0.004158 | dCMP deaminase, mRNA |
| BC001669.1 | 20 | 7 | 6.44E−06 | Similar to oxidase (cytochrome c) assembly 1-like, |
| | | | | clone MGC: 2171 |
| BC001873.1 | 11 | 2 | 0.0093506 | hairy/enhancer-of-split related with YRPW motif 1, |
| | | | | mRNA |
| BC001907.1 | 18 | 8 | 0.0010998 | hypothetical protein MGC2650 |
| BC001917.1 | 11 | 2 | 0.0028678 | malate dehydrogenase 2, NAD (mitochondrial), |
| | | | | mRNA |
| BC002493.1 | 17 | 9 | 0.0093506 | cDNA clone MGC: 2575 IMAGE: 3051226 |
| BC002677.1 | 16 | 3 | 0.0047651 | hypothetical protein, clone MGC: 3375 |
| | | | | IMAGE: 3609357 |
| BC002880.1 | 16 | 2 | 8.32E−06 | cysteinyl-tRNA synthetase, clone MGC: 11246 |
| BC002955.1 | 16 | 6 | 0.0047651 | ubiquitin specific peptidase 2, transcript variant 1, |
| | | | | mRNA |
| BC003065.1 | 16 | 5 | 0.0006159 | cyclin-dependent kinase 2, |
| BC003132.1 | 17 | 8 | 0.0093506 | nuclear distribution gene C homolog (A. nidulans) |
| BC003168.1 | 13 | 3 | 0.0047651 | oxysterol binding protein-like 10 |
| BC004271.1 | 14 | 4 | 0.0018205 | carnosinase 1 |
| BC004514.1 | 8 | 1 | 0.009828 | hypothetical protein FLJ12584 |
| BC005297.1 | 9 | 1 | 0.0041809 | kynurenine 3-monooxygenase (kynurenine 3- |
| | | | | hydroxylase), mRNA |
| BC005332.1 | 18 | 3 | 1.68E−06 | cDNA clone MGC: 12418 IMAGE: 3934658, complete |
| | | | | cds |
| BC006105.1 | 10 | 0 | 0.000218 | chromosome 6 open reading frame 134, mRNA |
| BC007363.1 | 15 | 6 | 0.0124209 | clone MGC: 16138 IMAGE: 3630050 |
| BC007411.2 | 20 | 11 | 0.001638 | diaphanous homolog 1 (Drosophila) |
| BC007560.1 | 9 | 0 | 0.0154752 | LIM and SH3 protein 1, mRNA |
| BC007581.1 | 8 | 0 | 0.001638 | aldehyde dehydrogenase 4 family, member A1, |
| | | | | transcript variant P5CDhL, mRNA |
| BC007872.1 | 19 | 9 | 0.009828 | thymidine kinase 1, soluble |
| BC009189.1 | 12 | 3 | 0.0112387 | CGI-39 protein; cell death-regulatory protein |
| | | | | GRIM19 |
| BC009250.1 | 17 | 6 | 0.0005323 | nucleolar GTPase |
| BC009696.1 | 14 | 3 | 0.0005323 | interferon induced transmembrane protein 2 (1-8D), |
| | | | | mRNA |
| BC009712.1 | 8 | 1 | 0.009828 | Similar to ATP-binding cassette, sub-family D (ALD), |
| | | | | member 3 |
| BC009894.2 | 17 | 5 | 0.0093506 | 3′-phosphoadenosine 5′-phosphosulfate synthase 2 |
| BC010176.1 | 9 | 1 | 0.0041809 | clone MGC: 20533 IMAGE: 3342874, |
| BC010959.1 | 11 | 2 | 0.0028678 | BCL2/adenovirus E1B 19 kDa interacting protein 1, |
| | | | | transcript variant BNIP1, mRNA |
| BC011710.2 | 10 | 1 | 0.0069071 | hypoxia-inducible factor prolyl 4-hydroxylase |
| BC011811.1 | 10 | 1 | 0.001671 | clone MGC: 20260 IMAGE: 3028747 |
| BC011885.1 | 9 | 1 | 0.0041809 | eukaryotic translation initiation factor (eIF) 2A, |
| | | | | mRNA |
| BC012109.1 | 19 | 10 | 0.001671 | homer homolog 2 (Drosophila) |
| BC012576.1 | 15 | 3 | 0.0001642 | Unknown (protein for MGC: 13472) |
| BC012609.1 | 9 | 1 | 0.0154752 | serpin peptidase inhibitor, clade B (ovalbumin), |
| | | | | member 2, mRNA |
| BC012783.2 | 12 | 1 | 0.0010998 | cDNA clone IMAGE: 3949276 |
| BC012876.1 | 18 | 4 | 8.32E−06 | clone MGC: 17259 IMAGE: 4149333 |
| BC014037.1 | 16 | 5 | 0.0018205 | Similar to serum/glucocorticoid regulated kinase 2 |
| BC014271.2 | 18 | 4 | 8.32E−06 | endoglin (Osler-Rendu-Weber syndrome 1), mRNA |
| BC014667.1 | 20 | 9 | 7.27E−05 | immunoglobulin heavy constant gamma 1 (G1m |
| | | | | marker), mRNA |
| BC014889.1 | 13 | 2 | 0.0015282 | requiem, apoptosis response zinc finger gene |
| BC015684.2 | 14 | 3 | 0.0005323 | Similar to Sjogren syndrome antigen A1 (52 kD, |
| | | | | ribonucleoprotein autoantigen SS-A/Ro) |
| BC015833.1 | 19 | 4 | 1.10E−06 | cDNA clone MGC: 27152 IMAGE: 4691630, complete |
| | | | | cds |
| BC015848.1 | 11 | 0 | 7.27E−05 | chromosome 17 open reading frame 25, mRNA |
| BC016380.1 | 18 | 4 | 8.32E−06 | cDNA clone MGC: 27376 IMAGE: 4688477, complete |
| | | | | cds |
| BC016381.1 | 15 | 3 | 0.0001642 | cDNA clone MGC: 27378 IMAGE: 4688865, complete |
| | | | | cds |
| BC016789.1 | 15 | 3 | 0.0051934 | glycine-N-acyltransferase-like 2, mRNA |
| BC017115.1 | 8 | 1 | 0.009828 | Unknown (protein for MGC: 16813) |
| BC017237.1 | 6 | 0 | 0.010098 | Similar to syntaxin 10 |
| BC017344.1 | 11 | 1 | 0.0006238 | Similar to hypothetical protein FLJ23469 |
| BC017865.1 | 20 | 10 | 0.000218 | Fc fragment of IgG, low affinity IIIa, receptor |
| | | | | (CD16a), mRNA |
| BC017959.1 | 7 | 0 | 0.004158 | hypothetical protein FLJ22555, mRNA |
| BC018749.1 | 8 | 0 | 0.001638 | immunoglobulin lambda variable 2-14, mRNA |
| BC019337.1 | 19 | 3 | 2.03E−07 | immunoglobulin heavy constant gamma 1 (G1m |
| | | | | marker), mRNA |
| BC020622.1 | 9 | 2 | 0.0154752 | zinc finger, A20 domain containing 1, mRNA, |
| | | | | complete cds. |
| BC020658.1 | 9 | 0 | 0.0041809 | transmembrane protein 40, mRNA |
| BC020962.1 | 13 | 1 | 6.85E−05 | similar to glucosamine-6-sulfatases |
| BC020985.1 | 16 | 5 | 0.0018205 | gi\|21594102 Unknown (protein for MGC: 9724) |
| BC021551.1 | 11 | 1 | 0.0006238 | hypothetical protein FLJ14639 |
| BC022098.1 | 18 | 4 | 8.32E−06 | cDNA clone MGC: 31944 IMAGE: 4878869, complete |
| | | | | cds |
| BC022362.1 | 19 | 6 | 1.97E−05 | cDNA clone MGC: 23888 IMAGE: 4704496, complete |
| | | | | cds |
| BC022454.2 | 11 | 1 | 0.0006238 | transient receptor potential cation channel, subfamily |
| | | | | M, member 3 |
| BC024289.1 | 15 | 2 | 3.43E−05 | cDNA clone MGC: 39273 IMAGE: 5440834, complete |
| | | | | cds |
| BC024291.1 | 12 | 3 | 0.0039558 | Similar to serine/threonine kinase 29 |
| BC025314.1 | 18 | 4 | 8.32E−06 | immunoglobulin heavy constant gamma 1 (G1m |
| | | | | marker), mRNA |
| BC025389.1 | 11 | 2 | 0.0028678 | hypothetical protein MGC26605 |
| BC027486.1 | 8 | 0 | 0.001638 | cDNA clone MGC: 34907 IMAGE: 5104096, complete |
| | | | | cds |
| BC028039.1 | 16 | 3 | 4.38E−05 | hypothetical protein MGC39900 |
| BC028728.1 | 11 | 0 | 7.27E−05 | Similar to putative ion channel protein |
| | | | | CATSPER2, clone MGC: 33346 IMAGE: 4828636, |
| | | | | mRNA, complete cds. |
| BC028840.1 | 6 | 0 | 0.010098 | hypothetical protein DKFZp566D1346 |
| BC029054.1 | 16 | 2 | 8.32E−06 | PDZ domain containing 7, mRNA |
| BC029444.1 | 19 | 4 | 1.10E−06 | cDNA clone MGC: 32714 IMAGE: 4692138, complete |
| | | | | cds |
| BC030814.1 | 16 | 2 | 8.32E−06 | immunoglobulin kappa variable 1-5, mRNA |
| BC030983.1 | 17 | 2 | 1.68E−06 | immunoglobulin lambda constant 1 (Mcg marker), |
| | | | | mRNA |
| BC030984.1 | 16 | 1 | 1.10E−06 | cDNA clone MGC: 32654 IMAGE: 4701898, complete |
| | | | | cds |
| BC031074.1 | 19 | 5 | 5.01E−06 | poly (ADP-ribose) polymerase family, member 16, |
| | | | | mRNA |
| BC031592.1 | 10 | 1 | 0.001671 | serpin peptidase inhibitor, clade F (alpha-2 |
| | | | | antiplasmin, pigment epithelium derived factor), |
| | | | | member 2, mRNA |
| BC031966.1 | 12 | 1 | 0.0002158 | cDNA clone MGC: 43036 IMAGE: 4839025 |
| BC032451.1 | 20 | 8 | 2.25E−05 | cDNA clone MGC: 40426 IMAGE: 5178085, complete |
| | | | | cds |
| BC032452.1 | 18 | 5 | 3.43E−05 | immunoglobulin lambda constant 1 (Mcg marker), |
| | | | | mRNA |
| BC033035.1 | 15 | 5 | 0.0019238 | similar to hypothetical protein, clone MGC: 33355 |
| | | | | IMAGE: 4839231 |
| BC033178.1 | 20 | 3 | 1.28E−08 | immunoglobulin heavy constant gamma 3 (G3m |
| | | | | marker), mRNA |
| BC034141.1 | 20 | 7 | 6.44E−06 | immunoglobulin kappa constant, mRNA |
| BC034146.1 | 16 | 8 | 0.0112387 | cDNA clone MGC: 32764 IMAGE: 4618950, complete |
| | | | | cds |
| BC034247.1 | 13 | 1 | 0.0003858 | chromosome 9 open reading frame 105, mRNA |
| BC035911.1 | 12 | 1 | 0.0010998 | DEAD (Asp-Glu-Ala-Asp) box polypeptide 55, |
| | | | | mRNA, complete cds. |
| BC036723.1 | 16 | 3 | 4.38E−05 | Fc fragment of IgG, low affinity IIIa, receptor |
| | | | | (CD16a), mRNA |
| BC038713.1 | 11 | 2 | 0.0093506 | pleckstrin homology, Sec7 and coiled-coil domains 2 |
| | | | | (cytohesin-2), transcript variant 1, mRNA |
| BC039814.1 | 17 | 4 | 4.38E−05 | zinc finger protein 265, transcript variant 2, mRNA |
| BC039904.1 | 15 | 5 | 0.0051934 | histone deacetylase 4, mRNA |
| BC040656.1 | 15 | 5 | 0.0019238 | leucine rich repeat containing 3B |
| BC041037.1 | 13 | 0 | 6.44E−06 | immunoglobulin heavy constant mu, mRNA |
| BC044584.1 | 16 | 8 | 0.0112387 | DnaJ (Hsp40) homolog, subfamily C, member 4, |
| | | | | mRNA |
| BC048125.1 | 10 | 1 | 0.001671 | hypothetical protein FLJ32800, mRNA |
| BC051382.1 | 9 | 0 | 0.0041809 | hypothetical protein MGC5987 |
| BC051762.1 | 13 | 3 | 0.0015282 | chromosome 20 open reading frame 96 |
| BC051885.1 | 20 | 10 | 0.0006143 | chromosome 14 open reading frame 106, mRNA, |
| | | | | complete cds. |
| BC053656.1 | 17 | 3 | 9.69E−06 | EGF-like repeats and discoidin I-like domains 3, |
| | | | | mRNA |
| BC053664.1 | 14 | 3 | 0.0005323 | complete cds. |
| BC053984.1 | 18 | 4 | 8.32E−06 | cDNA clone MGC: 59926 IMAGE: 5480266, complete |
| | | | | cds |
| BC056256.1 | 18 | 3 | 1.68E−06 | immunoglobulin kappa constant, mRNA |
| BC057770.1 | 10 | 0 | 0.000218 | solute carrier family 27 (fatty acid transporter), |
| | | | | member 2, mRNA |
| BC064367.1 | 17 | 9 | 0.0093506 | sterile alpha motif domain containing 6, mRNA |
| BC064945.1 | 9 | 1 | 0.0154752 | SCY1-like 1 binding protein 1, mRNA |
| CTL2122 | 16 | 4 | 0.00018 | RNA POLYMERASE |
| CTL2130 | 17 | 2 | 1.68E−06 | Proteinase-3 |
| CTL2134 | 18 | 6 | 0.0001222 | dsDNA |
| CTL2137 | 13 | 3 | 0.0015282 | La/SS-B |
| CTL2144 | 16 | 4 | 0.00018 | TRANSGLUTAMINASE |
| NM_000023.1 | 13 | 3 | 0.0015282 | sarcoglycan, alpha (50 kDa dystrophin-associated |
| | | | | glycoprotein) (SGCA), mRNA |
| NM_000137.1 | 16 | 7 | 0.0047651 | fumarylacetoacetate hydrolase |
| | | | | (fumarylacetoacetase) (FAH), mRNA |
| NM_001002018.1 | 18 | 11 | 0.0154752 | host cell factor C1 regulator 1 (XPO1 dependant) |
| | | | | (HCFC1R1), transcript variant 3, mRNA |
| NM_001203.1 | 20 | 11 | 0.0006143 | bone morphogenetic protein receptor, type IB |
| | | | | (BMPR1B), mRNA |
| NM_001258.1 | 12 | 2 | 0.0010998 | cyclin-dependent kinase 3 (CDK3), mRNA |
| NM_001449.2 | 18 | 3 | 1.68E−06 | four and a half LIM domains 1 (FHL1) |
| NM_001769.2 | 10 | 1 | 0.001671 | CD9 antigen (p24) (CD9), mRNA |
| NM_001892.2 | 6 | 0 | 0.010098 | casein kinase 1, alpha 1 (CSNK1A1), |
| NM_002082.1 | 16 | 6 | 0.0112387 | G protein-coupled receptor kinase 6 (GRK6) |
| NM_002362.2 | 6 | 0 | 0.010098 | melanoma antigen, family A, 4 (MAGEA4) |
| NM_002395.2 | 12 | 4 | 0.0112387 | malic enzyme 1, NADP(+)-dependent, cytosolic |
| | | | | (ME1), mRNA |
| NM_002431.1 | 15 | 6 | 0.0051934 | menage a trois 1 (CAK assembly factor) (MNAT1) |
| NM_002436.2 | 6 | 0 | 0.010098 | membrane protein, palmitoylated 1, 55 kDa (MPP1), |
| | | | | mRNA |
| NM_002576.2 | 11 | 2 | 0.0028678 | p21/Cdc42/Rac1-activated kinase 1 (STE20 |
| | | | | homolog, yeast) (PAK1) |
| NM_002578.1 | 20 | 12 | 0.001638 | p21 (CDKN1A)-activated kinase 3 (PAK3) |
| NM_002613.1 | 12 | 3 | 0.0039558 | 3-phosphoinositide dependent protein kinase-1 |
| | | | | (PDPK1) |
| NM_002744.2 | 6 | 0 | 0.010098 | protein kinase C, zeta (PRKCZ) |
| NM_002754.3 | 20 | 7 | 2.25E−05 | mitogen-activated protein kinase 13 (MAPK13), |
| | | | | mRNA |
| NM_002774.2 | 13 | 1 | 6.85E−05 | kallikrein 6 (neurosin, zyme) (KLK6) |
| NM_002963.2 | 13 | 3 | 0.0015282 | S100 calcium binding protein A7 (psoriasin 1) |
| | | | | (S100A7), mRNA |
| NM_003049.1 | 11 | 2 | 0.0093506 | solute carrier family 10 (sodium/bile acid |
| | | | | cotransporter family), member 1 (SLC10A1), mRNA |
| NM_003315.1 | 11 | 2 | 0.0028678 | DnaJ (Hsp40) homolog, subfamily C, member 7 |
| | | | | (DNAJC7), mRNA |
| NM_003476.2 | 12 | 2 | 0.0010998 | cysteine and glycine-rich protein 3 (cardiac LIM |
| | | | | protein) (CSRP3), mRNA |
| NM_003582.1 | 12 | 4 | 0.0112387 | dual-specificity tyrosine-(Y)-phosphorylation |
| | | | | regulated kinase 3 (DYRK3) |
| NM_003792.1 | 9 | 1 | 0.0041809 | endothelial differentiation-related factor 1 (EDF1) |
| NM_003942.1 | 10 | 0 | 0.000218 | ribosomal protein S6 kinase, 90 kDa, polypeptide 4 |
| | | | | (RPS6KA4) |
| NM_004073.2 | 14 | 3 | 0.0005323 | polo-like kinase 3 (Drosophila) (PLK3), mRNA |
| NM_004078.1 | 12 | 2 | 0.0010998 | cysteine and glycine-rich protein 1 (CSRP1), mRNA |
| NM_004089.1 | 13 | 3 | 0.0047651 | delta sleep inducing peptide, immunoreactor (DSIPI) |
| NM_004181.2 | 14 | 3 | 0.0005323 | ubiquitin carboxyl-terminal esterase L1 (ubiquitin |
| | | | | thiolesterase) (UCHL1) |
| NM_004722.2 | 8 | 1 | 0.009828 | adaptor-related protein complex 4, mu 1 subunit |
| | | | | (AP4M1), mRNA |
| NM_004732.1 | 17 | 5 | 0.0005323 | potassium voltage-gated channel, shaker-related |
| | | | | subfamily, beta member 3 (KCNAB3) |
| NM_004759.2 | 12 | 2 | 0.0010998 | mitogen-activated protein kinase-activated protein |
| | | | | kinase 2 (MAPKAPK2), transcript variant 1 |
| NM_004881.1 | 9 | 2 | 0.0154752 | quinone oxidoreductase homolog (PIG3) |
| NM_004935.1 | 16 | 7 | 0.0112387 | cyclin-dependent kinase 5 (CDK5) |
| NM_005157.2 | 16 | 4 | 0.0047651 | v-abl Abelson murine leukemia viral oncogene |
| | | | | homolog 1 (ABL1), transcript variant a |
| NM_005347.2 | 9 | 2 | 0.0154752 | heat shock 70 kDa protein 5 (glucose-regulated |
| | | | | protein, 78 kDa) (HSPA5), mRNA |
| NM_005435.2 | 18 | 10 | 0.0069071 | Rho guanine nucleotide exchange factor (GEF) 5 |
| | | | | (ARHGEF5) |
| NM_005522.3 | 17 | 3 | 9.69E−06 | homeo box A1 (HOXA1), transcript variant 1 |
| NM_005697.3 | 12 | 1 | 0.0002158 | secretory carrier membrane protein 2 (SCAMP2), |
| | | | | mRNA |
| NM_006002.2 | 8 | 0 | 0.001638 | ubiquitin carboxyl-terminal esterase L3 (ubiquitin |
| | | | | thiolesterase) (UCHL3) |
| NM_006552.1 | 8 | 0 | 0.009828 | secretoglobin, family 1D, member 1 (SCGB1D1), |
| | | | | mRNA |
| NM_006775.1 | 9 | 2 | 0.0154752 | quaking homolog, KH domain RNA binding (mouse) |
| | | | | (QKI), transcript variant 1, mRNA |
| NM_006869.1 | 20 | 13 | 0.004158 | centaurin, alpha 1 (CENTA1), mRNA |
| NM_012097.2 | 11 | 0 | 0.0028678 | ADP-ribosylation factor-like 5 (ARL5), transcript |
| | | | | variant 1 |
| NM_012101.2 | 13 | 4 | 0.0047651 | tripartite motif-containing 29 (TRIM29), transcript |
| | | | | variant 1, mRNA |
| NM_012241.2 | 13 | 1 | 6.85E−05 | sirtuin (silent mating type information regulation 2 |
| | | | | homolog) 5 (S. cerevisiae) (SIRT5), transcript variant |
| | | | | 1, mRNA |
| NM_013254.2 | 15 | 7 | 0.0124209 | TANK-binding kinase 1 (TBK1), mRNA |
| NM_013322.2 | 16 | 5 | 0.0018205 | sorting nexin 10 (SNX10), mRNA |
| NM_013410.1 | 12 | 1 | 0.0039558 | adenylate kinase 3 (AK3) |
| NM_014046.2 | 8 | 0 | 0.001638 | mitochondrial ribosomal protein S18B (MRPS18B), |
| | | | | nuclear gene encoding mitochondrial protein, mRNA |
| NM_014188.2 | 6 | 0 | 0.010098 | HSPC182 protein (HSPC182), mRNA |
| NM_014251.1 | 11 | 3 | 0.0093506 | solute carrier family 25, member 13 (citrin) |
| | | | | (SLC25A13), mRNA |
| NM_016006.1 | 15 | 5 | 0.0124209 | comparative gene identification 58 (CGI58) |
| NM_016052.1 | 12 | 2 | 0.0010998 | CGI-115 protein (CGI-115) |
| NM_016091.1 | 9 | 1 | 0.0041809 | eukaryotic translation initiation factor 3, subunit 6 |
| | | | | interacting protein (EIF3S6IP) |
| NM_016207.2 | 11 | 0 | 7.27E−05 | cleavage and polyadenylation specific factor 3, |
| | | | | 73 kDa (CPSF3), mRNA |
| NM_017503.2 | 7 | 0 | 0.004158 | surfeit 2 (SURF2), mRNA |
| NM_017811.2 | 8 | 1 | 0.009828 | ubiquitin-conjugating enzyme E2R 2 (UBE2R2) |
| NM_018129.1 | 9 | 2 | 0.0154752 | pyridoxine 5′-phosphate oxidase (PNPO), mRNA |
| NM_018184.1 | 18 | 9 | 0.0154752 | ADP-ribosylation factor-like 10C (ARL10C) |
| NM_018679.2 | 10 | 1 | 0.0069071 | t-complex 11 (mouse) (TCP11), mRNA |
| NM_019103.1 | 7 | 0 | 0.004158 | hypothetical protein LOC55954 |
| NM_020367.2 | 10 | 1 | 0.001671 | chromosome 12 open reading frame 6 (C12orf6) |
| NM_020381.2 | 9 | 0 | 0.0006143 | chromosome 6 open reading frame 210 (C6orf210), |
| | | | | mRNA |
| NM_020444.2 | 18 | 10 | 0.0069071 | KIAA1191 protein (KIAA1191), mRNA |
| NM_020547.1 | 9 | 1 | 0.0041809 | anti-Mullerian hormone receptor, type II (AMHR2) |
| NM_020929.1 | 15 | 3 | 0.0124209 | netrin-G1 ligand (NGL-1), mRNA |
| NM_021117.1 | 8 | 0 | 0.001638 | cryptochrome 2 (photolyase-like) (CRY2), mRNA |
| NM_021146.2 | 11 | 2 | 0.0028678 | angiopoietin-like 7 (ANGPTL7), mRNA |
| NM_021254.1 | 9 | 2 | 0.0154752 | chromosome 21 open reading frame 59 (C21orf59), |
| | | | | mRNA |
| NM_021709.1 | 13 | 2 | 0.0015282 | CD27-binding (Siva) protein (SIVA), transcript |
| | | | | variant 2, mRNA |
| NM_021945.1 | 13 | 2 | 0.0015282 | hypothetical protein FLJ22174 |
| NM_022777.1 | 11 | 1 | 0.0006238 | RAB, member RAS oncogene family-like 5 (RABL5), |
| | | | | mRNA |
| NM_024041.1 | 19 | 8 | 0.0002158 | sodium channel modifier 1 (SCNM1) |
| NM_024096.1 | 9 | 1 | 0.0041809 | XTP3-transactivated protein A (XTP3TPA), mRNA |
| NM_024348.2 | 8 | 1 | 0.009828 | dynactin 3 (p22) (DCTN3), transcript variant 2, |
| | | | | mRNA |
| NM_024419.2 | 7 | 0 | 0.004158 | phosphatidylglycerophosphate synthase (PGS1) |
| NM_024749.1 | 16 | 6 | 0.0018205 | hypothetical protein FLJ12505 |
| NM_024770.1 | 12 | 3 | 0.0112387 | hypothetical protein FLJ13984 (FLJ13984), mRNA |
| NM_024786.1 | 15 | 6 | 0.0124209 | zinc finger, DHHC domain containing 11 |
| | | | | (ZDHHC11), mRNA |
| NM_024893.1 | 12 | 0 | 2.25E−05 | chromosome 20 open reading frame 39 (C20orf39), |
| | | | | mRNA |
| NM_030773.1 | 10 | 2 | 0.0069071 | tubulin, beta 1 (TUBB1) |
| NM_032146.2 | 17 | 9 | 0.0093506 | ADP-ribosylation factor-like 6 (ARL6) |
| NM_032731.2 | 13 | 1 | 6.85E−05 | thioredoxin-like 5 (TXNL5), mRNA |
| NM_032855.1 | 18 | 5 | 3.43E−05 | hematopoietic SH2 protein (HSH2) |
| NM_052844.1 | 11 | 1 | 0.0006238 | hypothetical protein MGC20486 |
| NM_052848.1 | 8 | 1 | 0.009828 | hypothetical protein, mRNA |
| NM_052877.1 | 20 | 9 | 7.27E−05 | mediator of RNA polymerase II transcription, subunit |
| | | | | 8 homolog (yeast) (MED8) |
| NM_058163.1 | 12 | 4 | 0.0112387 | hypothetical protein DT1P1A10 (DT1P1A10), mRNA |
| NM_058217.1 | 15 | 6 | 0.0051934 | RAD51 homolog C (S. cerevisiae) (RAD51C), |
| | | | | transcript variant 3 |
| NM_138355.1 | 14 | 3 | 0.0128224 | secernin 2 (Ses2) |
| NM_138432.1 | 12 | 2 | 0.0010998 | serine dehydratase related sequence 1 (SDS-RS1) |
| NM_138455.1 | 16 | 5 | 0.0018205 | collagen triple helix repeat containing 1 |
| NM_138470.1 | 13 | 1 | 0.0047651 | hypothetical protein BC008131 (LOC142937) |
| NM_139240.2 | 11 | 1 | 0.0006238 | LOC92346 (LOC92346), mRNA |
| NM_145063.1 | 20 | 14 | 0.010098 | chromosome 6 open reading frame 130 (C6orf130) |
| NM_145109.1 | 16 | 6 | 0.0112387 | mitogen-activated protein kinase kinase 3 |
| | | | | (MAP2K3), transcript variant B, mRNA |
| NM_145792.1 | 12 | 2 | 0.0010998 | microsomal glutathione S-transferase 1 (MGST1), |
| | | | | transcript variant 1a |
| NM_148975.1 | 10 | 2 | 0.0069071 | membrane-spanning 4-domains, subfamily A, |
| | | | | member 4 (MS4A4A), transcript variant 2, mRNA |
| NM_152421.2 | 6 | 0 | 0.010098 | hypothetical protein, mRNA |
| NM_152690.1 | 12 | 1 | 0.0002158 | dolichyl-phosphate mannosyltransferase polypeptide |
| | | | | 2, regulatory subunit (DPM2), transcript variant 2 |
| NM_152772.1 | 16 | 6 | 0.0112387 | hypothetical protein, mRNA |
| NM_153215.1 | 19 | 5 | 1.97E−05 | hypothetical protein FLJ38608 (FLJ38608), mRNA |
| NM_173519.1 | 17 | 6 | 0.0093506 | hypothetical protein, mRNA |
| NM_175907.3 | 20 | 8 | 2.25E−05 | zinc binding alcohol dehydrogenase, domain |
| | | | | containing 2 (ZADH2), mRNA |
| NM_178496.2 | 19 | 7 | 0.001671 | similar to BcDNA:GH11415 gene product |
| | | | | (LOC151963), mRNA |
| NM_178832.2 | 16 | 4 | 0.0112387 | chromosome 10 open reading frame 83 (C10orf83), |
| | | | | mRNA |
| NM_181738.1 | 12 | 2 | 0.0010998 | peroxiredoxin 2 (PRDX2), nuclear gene encoding |
| | | | | mitochondrial protein, transcript variant 3, mRNA |
| NM_183059.1 | 13 | 4 | 0.0047651 | chromosome 1 open reading frame 36 (C1orf36), |
| | | | | mRNA |
| NM_197964.1 | 14 | 6 | 0.0128224 | hypothetical protein HSPC268 (HSPC268), mRNA |
| NM_198490.1 | 9 | 1 | 0.0154752 | RAB43, member RAS oncogene family (RAB43), |
| | | | | mRNA |
| NP_005219.2 | 15 | 7 | 0.0124209 | epidermal growth factor receptor (erthroblastic |
| | | | | leukemia viral (v-erb-b) oncogene homolog, avian) |
| | | | | (EGFR), mutant isoform L861Q |

TABLE 13
A list of proteins that were bound by an antibody from ANCA patient sera but
not RA patients.

| Genbank ID | | | | |
|---|---|---|---|---|
| number of | | | | |
| nucleic acid | | | | |
| coding for | ANCA | RA | | |
| the protein | Count | Count | p-value | Name or description |
| BC000442.1 | 0 | 13 | 0.001142 | serine/threonine kinase 12 |
| BC000463.1 | 1 | 19 | 0.00017 | splicing factor 3b, subunit 3, 130 kD |
| BC001709.1 | 0 | 9 | 0.016771 | NAD kinase, mRNA |
| BC002880.1 | 0 | 15 | 0.0002 | cysteinyl-tRNA synthetase |
| BC003065.1 | 2 | 18 | 0.000701 | cyclin-dependent kinase 2 |
| BC003168.1 | 1 | 13 | 0.009495 | oxysterol binding protein-like 10 |
| BC005332.1 | 0 | 11 | 0.004855 | cDNA clone MGC: 12418 IMAGE: 3934658, |
| | | | | complete cds |
| BC006105.1 | 2 | 16 | 0.005343 | chromosome 6 open reading frame 134, mRNA |
| BC006550.1 | 0 | 10 | 0.009224 | RNA binding motif protein, X chromosome |
| BC007363.1 | 0 | 14 | 0.0005 | clone MGC: 16138 IMAGE: 3630050 |
| BC007581.1 | 0 | 14 | 0.0005 | aldehyde dehydrogenase 4 family, member A1, |
| | | | | transcript variant P5CDhL, mRNA |
| BC009894.2 | 1 | 16 | 0.000862 | 3′-phosphoadenosine 5′-phosphosulfate synthase 2 |
| BC011792.1 | 0 | 10 | 0.009224 | clone MGC: 19561 IMAGE: 4300082 |
| BC012783.2 | 0 | 9 | 0.016771 | cDNA clone IMAGE: 3949276 |
| BC020962.1 | 0 | 14 | 0.0005 | similar to glucosamine-6-sulfatases |
| BC021121.1 | 3 | 20 | 0.000177 | mRNA, complete cds. |
| BC022098.1 | 0 | 9 | 0.016771 | cDNA clone MGC: 31944 IMAGE: 4878869, |
| | | | | complete cds |
| BC025345.1 | 2 | 19 | 0.00017 | mRNA similar to LOC149651 (cDNA clone |
| | | | | MGC: 39393 IMAGE: 4862156), complete cds |
| BC028040.1 | 4 | 20 | 0.001061 | 2′,3′-cyclic nucleotide 3′ phosphodiesterase, mRNA |
| BC029444.1 | 0 | 11 | 0.004855 | cDNA clone MGC: 32714 IMAGE: 4692138, |
| | | | | complete cds |
| BC030814.1 | 2 | 16 | 0.005343 | immunoglobulin kappa variable 1-5, mRNA |
| BC030983.1 | 3 | 19 | 0.001099 | immunoglobulin lambda constant 1 (Mcg marker), |
| | | | | mRNA |
| BC030984.1 | 3 | 19 | 0.001099 | cDNA clone MGC: 32654 IMAGE: 4701898, |
| | | | | complete cds |
| BC031650.1 | 1 | 13 | 0.009495 | clone MGC: 35144 IMAGE: 5169239 |
| BC032452.1 | 3 | 17 | 0.010263 | immunoglobulin lambda constant 1 (Mcg marker), |
| | | | | mRNA |
| BC032485.1 | 3 | 17 | 0.010263 | hypothetical protein FLJ30473 |
| BC033178.1 | 5 | 20 | 0.005305 | immunoglobulin heavy constant gamma 3 (G3m |
| | | | | marker), mRNA |
| BC033856.1 | 4 | 19 | 0.005482 | Similar to RIKEN cDNA 3110040D16 gene, |
| | | | | cloneMGC: 45395 IMAGE: 5123380, mRNA, |
| | | | | complete cds. |
| BC034141.1 | 2 | 17 | 0.002135 | immunoglobulin kappa constant, mRNA |
| BC034247.1 | 0 | 9 | 0.016771 | chromosome 9 open reading frame 105, mRNA |
| BC039814.1 | 1 | 14 | 0.004698 | zinc finger protein 265, transcript variant 2, mRNA |
| BC044584.1 | 0 | 14 | 0.0005 | DnaJ (Hsp40) homolog, subfamily C, member 4, |
| | | | | mRNA |
| BC053664.1 | 1 | 12 | 0.017848 | complete cds. |
| BC056256.1 | 2 | 15 | 0.011617 | immunoglobulin kappa constant, mRNA |
| BC064367.1 | 0 | 15 | 0.0002 | sterile alpha motif domain containing 6, mRNA |
| CTL2130 | 2 | 18 | 0.000701 | proteinase-3 |
| CTL2137 | 1 | 15 | 0.002128 | La/SS-B |
| NM_001001550.1 | 2 | 18 | 0.003888 | growth factor receptor-bound protein 10 (GRB10), |
| | | | | transcript variant 3, mRNA |
| NM_001203.1 | 0 | 16 | 7.14E−05 | bone morphogenetic protein receptor, type IB |
| | | | | (BMPR1B), mRNA |
| NM_001258.1 | 0 | 11 | 0.004855 | cyclin-dependent kinase 3 (CDK3), mRNA |
| NM_001280.1 | 3 | 18 | 0.003888 | cold inducible RNA binding protein (CIRBP), mRNA |
| NM_001449.2 | 1 | 18 | 8.64E−05 | four and a half LIM domains 1 |
| NM_001894.2 | 0 | 10 | 0.009224 | casein kinase 1, epsilon (CSNK1E) |
| NM_003582.1 | 1 | 15 | 0.002128 | dual-specificity tyrosine-(Y)-phosphorylation |
| | | | | regulated kinase 3 (DYRK3) |
| NM_003942.1 | 2 | 15 | 0.011617 | ribosomal protein S6 kinase, 90 kDa, polypeptide 4 |
| | | | | (RPS6KA4) |
| NM_004073.2 | 0 | 14 | 0.0005 | polo-like kinase 3 (Drosophila) (PLK3), mRNA |
| NM_004217.1 | 1 | 16 | 0.000862 | aurora kinase B (AURKB) |
| NM_004497.1 | 0 | 10 | 0.009224 | forkhead box A3 (FOXA3) |
| NM_004732.1 | 3 | 17 | 0.010263 | potassium voltage-gated channel, shaker-related |
| | | | | subfamily, beta member 3 (KCNAB3) |
| NM_004906.3 | 0 | 10 | 0.009224 | Wilms tumor 1 associated protein (WTAP), |
| | | | | transcript variant 1, mRNA |
| NM_006428.3 | 0 | 10 | 0.009224 | mitochondrial ribosomal protein L28 (MRPL28), |
| | | | | nuclear gene encoding mitochondrial protein, mRNA |
| NM_006869.1 | 0 | 19 | 1.81E−05 | centaurin, alpha 1 (CENTA1), mRNA |
| NM_012241.2 | 0 | 14 | 0.0005 | sirtuin (silent mating type information regulation 2 |
| | | | | homolog) 5 (S. cerevisiae) (SIRT5), transcript |
| | | | | variant 1, mRNA |
| NM_014481.2 | 2 | 16 | 0.005343 | APEX nuclease (apurinic/apyrimidinic |
| | | | | endonuclease) 2 (APEX2), nuclear gene encoding |
| | | | | mitochondrial protein, mRNA |
| NM_016584.2 | 0 | 9 | 0.016771 | interleukin 23, alpha subunit p19 (IL23A), mRNA |
| NM_017503.2 | 0 | 10 | 0.009224 | surfeit 2 (SURF2), mRNA |
| NM_018047.1 | 0 | 13 | 0.001142 | RNA binding motif protein 22 (RBM22), mRNA |
| NM_020381.2 | 2 | 15 | 0.011617 | chromosome 6 open reading frame 210 (C6orf210), |
| | | | | mRNA |
| NM_021117.1 | 0 | 9 | 0.016771 | cryptochrome 2 (photolyase-like) (CRY2), mRNA |
| NM_021945.1 | 2 | 16 | 0.005343 | hypothetical protein FLJ22174 (FLJ22174) |
| NM_024041.1 | 1 | 18 | 8.64E−05 | sodium channel modifier 1 |
| NM_052877.1 | 5 | 20 | 0.005305 | mediator of RNA polymerase II transcription, subunit |
| | | | | 8 homolog (yeast) (MED8) |
| NM_139240.2 | 0 | 10 | 0.009224 | LOC92346 (LOC92346), mRNA |
| NM_152376.2 | 0 | 10 | 0.009224 | UBX domain containing 3 (UBXD3), mRNA |
| NM_152697.2 | 0 | 14 | 0.004698 | hypothetical protein, mRNA |
| NM_153215.1 | 4 | 18 | 0.016438 | hypothetical protein FLJ38608 (FLJ38608), mRNA |
| NM_175907.3 | 1 | 17 | 0.000302 | zinc binding alcohol dehydrogenase, domain |
| | | | | containing 2 (ZADH2), mRNA |

### Example 5

This study utilized high-content protein microarrays comprised of more than 5,000 human proteins, including 25 known autoantigens, to evaluate immunological profiles across panels of serum samples derived from healthy donors and Systemic Lupus Erythemasosus (SLE) patients.

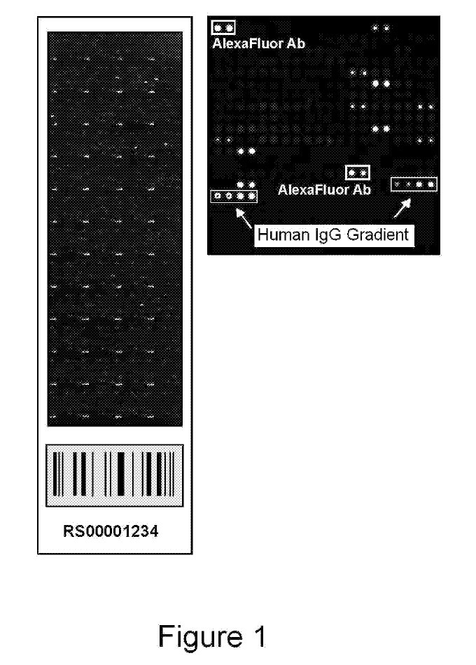

The microarrays were designed to include more than 5,000 recombinant human proteins, purified under non-denaturing conditions from a insect cell expression system. Most of the protein features included an N-terminal GST tag to facilitate protein purification as well as quality control assays designed to validate protein immobilization on the microarrays. In addition, more than 25 known autoantigens were integrated with the array features. These included autoantigens designated by the ARA as diagnostic for SLE in combination with other clinical symptoms (Table 14a). The arrays were spotted using contact printing technology, in which proteins were deposited as adjacent duplicates arranged in 48 individual subarrays, with each subarray including control elements designed to facilitate data acquisition and serve as indicators of assay performance (FIG. 1).

TABLE 14A
Annotated autoantigens included on the 5,000-protein microarray.
Autoantigens consistent with the ARA diagnostic criteria are indicated.

| | ARA |
|---|---|
| Autoantigen | Diagnostic |
| purified vimentin | |
| pyruvate dehydrogenase | |
| transglutaminase | |
| single stranded DNA | |
| double stranded DNA | X |
| unfractionated whole histone | |
| RNA polymerase (E. coli) | |
| cardiolipin | X |
| ribosomal RNA | X |
| Ro-52 | X |
| Jo-1 | |
| thyroglobulin | |
| Smith antigen (Sm) | X |
| RNP complex | X |
| histone H2a(f2a2) | |
| Centromere Protein B (CENP B) | |
| La/SS-B (La) | X |
| DNA Topoisomerase I (Scl-70; full | |
| length) | |
| U1-snRNP 68 Protein (68 kDa) | X |
| beta-2-glycoprotein 1 | |
| myeloperoxidase | |
| proteinase-3 | |
| ds plasmid DNA | X |
| myeloperoxidase | |
| proteinase-3 | |
| Cyclic citruillinated peptide | |

Three statistical approaches were applied in parallel to identify more than 230 candidate biomarkers for SLE (Table 14b). Independent expression and purification of these putative autoantigens was carried out in order to develop custom protein microarrays for use in validation studies. A global ranking scheme was developed for the >230 candidate SLE biomarkers through the use of a scoring system in which proteins were assigned a point for each of the specified threshold criteria they met. The scoring metric factored in a number of statistical parameters including Z-factor, M-statistics p-value, Signal Used difference, and Signal Used ratio, with 18 of the proteins generating the maximum score (Table 15). (It should be noted that the 18 proteins present in Table 15 are included in Table 3.)

TABLE 14B
Candidate biomarkers tested for presence in SLE patients
Database ID

BC000381.2
BC000979.2
BC001120.1
BC001129.1
BC001396.1
BC001907.1
BC001917.1
BC002733.2
BC002880.1
BC003132.1
BC004271.1
BC005248.1
BC006192.1
BC006376.1
BC006793.1
BC007581.1
BC007872.1
BC007888.1
BC008623.1
BC009623.1
BC009696.1
BC009762.2
BC009873.1
BC010947.1
BC011379.1
BC011668.1
BC011811.1
BC011842.2
BC011885.1
BC011888.1
BC012120.1
BC012575.1
BC012576.1
BC012783.2
BC012924.1
BC013073.1
BC013567.1
BC014244.1
BC014452.1
BC014949.1
BC015008.1
BC015497.1
BC015833.1
BC015904.1
BC016380.1
BC016764.1
BC016842.1
BC017114.1
BC017344.1
BC018749.1
BC018929.1
BC020597.1
BC020647.1
BC020962.1
BC022098.1
BC022325.1
BC022362.1
BC022454.2
BC023982.1
BC024289.1
BC024291.1
BC025281.1
BC025389.1
BC025996.2
BC027486.1
BC027607.1
BC028301.1
BC028728.1
BC029046.1
BC029054.1
BC029444.1
BC030219.1
BC030702.1
BC031966.1
BC032347.1
BC032451.1
BC032462.1
BC032485.1
BC032852.2
BC033035.1
BC033178.1
BC033856.1
BC034141.1
BC034146.1
BC034247.1
BC038105.2
BC038713.1
BC040656.1
BC041037.1
BC041157.1
BC042625.1
BC042864.1
BC048125.1
BC050428.1
BC051762.1
BC051885.1
BC052806.1
BC055314.1
BC056256.1
BC063275.1
BC063479.1
BC067446.1
BC067735.1
BC068460.1
NM_000801.2
NM_000993.2
NM_001014.2
NM_001029.2
NM_001106.2
NM_001124.1
NM_001219.2
NM_001280.1
NM_001449.2
NM_001501.1
NM_001616.2
NM_001697.1
NM_001769.2
NM_001894.2
NM_001896.1
NM_001896.2
NM_002053.1
NM_002129.2
NM_002287.2
NM_002362.2
NM_002436.2
NM_002443.2
NM_002576.2
NM_002578.1
NM_002613.1
NM_002754.3
NM_002774.2
NM_003130.1
NM_003295.1
NM_003463.2
NM_003476.2
NM_003583.2
NM_003621.1
NM_003792.1
NM_003897.2
NM_004089.1
NM_004181.2
NM_004214.3
NM_004383.1
NM_004578.2
NM_004582.2
NM_004596.1
NM_004645.1
NM_004656.2
NM_004765.2
NM_004881.1
NM_005368.1
NM_005441.2
NM_005522.3
NM_005558.2
NM_005697.3
NM_006002.2
NM_006169.1
NM_006205.1
NM_006251.4
NM_006298.2
NM_006374.2
NM_006388.2
NM_006607.1
NM_007008.1
NM_007162.1
NM_007240.1
NM_012163.1
NM_013375.2
NM_014176.1
NM_014685.1
NM_015640.1
NM_015987.2
NM_016091.1
NM_016289.2
NM_016355.3
NM_016360.1
NM_016483.3
NM_016505.2
NM_017495.3
NM_017503.2
NM_017588.1
NM_017811.2
NM_017838.2
NM_017846.3
NM_018032.2
NM_018047.1
NM_018129.1
NM_018457.1
NM_020239.2
NM_020317.2
NM_020661.1
NM_020664.3
NM_021146.2
NM_021254.1
NM_022777.1
NM_022787.2
NM_024041.1
NM_024096.1
NM_024114.1
NM_024749.1
NM_024893.1
NM_025055.2
NM_031412.1
NM_031465.2
NM_031910.2
NM_032042.2
NM_032146.2
NM_032345.1
NM_032350.3
NM_032855.1
NM_033642.1
NM_052848.1
NM_054016.1
NM_138414.1
NM_138419.1
NM_138771.1
NM_144982.1
NM_145020.1
NM_145063.1
NM_145315.2
NM_145792.1
NM_148975.1
NM_152430.1
NM_152638.2
NM_152690.1
NM_152769.1
NM_153207.2
NM_153332.2
NM_173519.1
NM_175907.3
NM_175923.2
NM_177996.1

TABLE 15
Candidate SLE biomarkers achieving the maximum score in a
ranking metric applied to protein microarray data.

| | Database ID | Description |
|---|---|---|
| 1 | NM_001106.2 | activin A receptor, type IIB |
| 2 | BC038105.2 | membrane protein, palmitoylated 7 |
| | | (MAGUK p55 subfamily member 7) |
| 3 | BC055314.1 | C2f protein |
| 4 | BC063275.1 | eukaryotic translation initiation factor 2C, 1 |
| 5 | NM_001616.2 | activin A receptor, type II |
| 6 | NM_020317.2 | hypothetical protein dJ465N24.2.1 |
| 7 | BC042625.1 | LUC7-like 2 |
| 8 | NM_145020.1 | hypothetical protein FLJ32743 |
| 9 | BC009873.1 | clone MGC: 16442 IMAGE: 3946787 |
| 10 | NM_022787.2 | nicotinamide nucleotide adenylyltransferase 1 |
| 11 | BC025996.2 | cDNA clone MGC: 26787 IMAGE: 4838986 |
| 12 | NM_004596.1 | small nuclear ribonucleoprotein polypeptide A |
| 13 | NM_018032.2 | LUC7-like |
| 14 | BC012924.1 | dual adaptor of phosphotyrosine and |
| | | 3-phosphoinositides |
| 15 | BC022325.1 | polyhomeotic like 3 |
| 16 | NM_015640.1 | PAI-1 mRNA-binding protein |
| 17 | NM_001014.2 | ribosomal protein S10 |
| 18 | NM_004765.2 | B-cell CLL/lymphoma 7C |

Luminex®-bead sets were prepared for validation studies using these 18 candidate SLE autoantigens. A validation rate of approximately 70% was observed across both microarray and Luminex X-MAP® technology platforms when the same set of disease and normal serum samples were used as probes. Improved discrimination between the two populations was observed when Principal Component Analysis was applied to data derived from 18 novel, protein microarray-defined proteins relative to autoantigens with annotated associated with SLE. Leave-one-out cross-validation analysis using support vector machine learning calculated a classification error rate of 3.3% for the array-defined candidate biomarkers, relative to an error rate of 13.3% calculated for the annotated SLE biomarkers. Taken together, this study provides the experimental and statistical framework to support the adoption of protein microarray technology as a tool for immunological profiling for disease biomarker discovery.

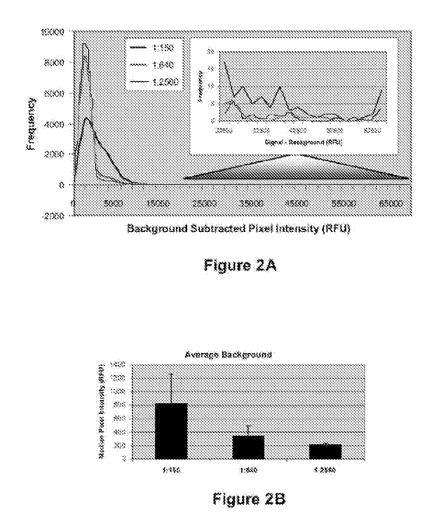

Diagnostic assays directed towards detection of the ARA-designated SLE autoantigens are typically performed at serum dilutions ranging from 1:10-1:100 to minimize false positive and false negative signals. Previous work on autoantigen arrays has suggested that this platform may be more sensitive, thus requiring a greater dilution factor to produce optimal signals and maximal dynamic range. To confirm this observation, a panel of 12 samples including serum from healthy individuals and SLE patients was evaluated on the high content human ProtoArray® at three dilutions: 1:150, 1:640, and 1:2560. Following the assays, high resolution images were obtained for each array and pixel intensity data was obtained corresponding to defined circular features and as well as local background. Histograms were generated for each sample representing the frequency with which background-subtracted signal intensity values were observed across the dynamic range. A representative signal distribution plot corresponding to one SLE sample is shown in FIG. 2. The majority of signals across all three dilutions were observed below 10,000 Relative Fluorescence Units (RFU); however, a significant increase in the number of array features giving rise to signals above 5,000 RFU were observed at the 1:150 dilution relative to the two higher dilutions, suggesting that larger dilutions may increase the likelihood of false negatives in the assay. Although signals observed above 20,000 RFU were fewer in total across all dilutions tested, a significantly greater number of array features gave rise to high intensity signals at the 1:150 dilution (FIG. 2, right panel). Median pixel intensity values corresponding to local background were calculated for each array across the three dilutions tested, and average background values across all arrays were calculated. The maximum measurable signal on the scanner used in these assays is 65,000 RFU. The average background values across the three dilutions tested differed by less than 4-fold, suggesting a similar available dynamic range (FIG. 2B). Based on these results, the 1:150 dilution was selected for use in profiling an expanded panel of serum samples. Twenty serum samples drawn from SLE patients, and ten sera drawn from healthy individuals were diluted 1:150 and profiled on the 5,000-protein microarrays. High resolution images were obtained on a fluorescent microarray scanner and pixel intensity data was captured through image analysis.

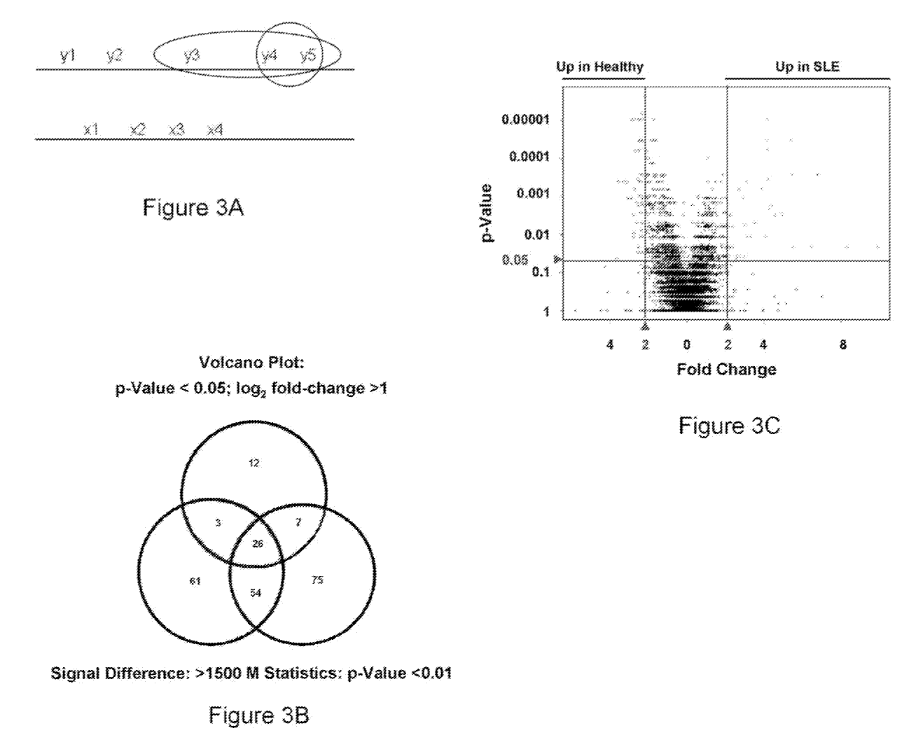

Three statistical approaches were applied to the data and the results of these analyses were compared. One of the methods employed in the analysis of the protein microarray data utilized M-statistics applied to quantile-normalized signal intensity data. This algorithm provides a count corresponding to the number of assays in one group for which a signal value for a specified protein is larger then the largest observed signal value for this protein in another group (FIG. 3A, red ellipse). Subsequent calculations specify the number of arrays in one population with signals arising from this protein that are larger than the second largest signal in the other population (FIG. 3A, violet ellipse), third largest etc., proceeding iteratively through the data set for all proteins on the array. The M “I” order statistic for the group y of size n_{y} compared to group x of size nₓ is given by:

$${M}_{i , above , between}^{y} = {\sum }_{k = 1}^{{n}_{y}} {1}_{\{ {y}_{k} > {x}_{( i )} + between \}} {1}_{\{ {y}_{k} > above \}}$$

where x₍ᵢ₎ is the i^{th} largest value of the group x, and above and between are the calculation parameters. The p-value is calculated as a probability of having an M value greater or equal then Mᵢ. The M statistic with the lowest p-value was selected, and the corresponding p-value was used to establish a threshold for selection of significant biomarker candidates. A second method utilized to analyze the SLE autoantibody profiles was the ‘volcano plot’, in which non-normalized signal intensity data is arranged along dimensions of biological and statistical significance. The first (horizontal) dimension represents the log-scale fold change between the two populations, and the second (vertical) axis represents the p-value for a t-test of differences between samples. The first axis indicates biological impact of the change; the second indicates the statistical evidence, or reliability of the change. The pixel intensity microarray data obtained from the SLE and healthy autoantibody profiling experiments was used in a volcano plot statistical approach in which p-Values were calculated using M-statistics. This analysis identified 48 proteins that resulted in a p-Value<0.05, and a log₂ fold-change>1 (FIG. 3B). It has been reported that p-Values computed using commonly used statistics including a two sample t-test, U- (Mann-Whitney) and M-statistics give rise to a largely similar rank order of array features. In the third analytical approach, a simple difference in background-subtracted signal values (Signal Used) was calculated from quantile-normalized signal intensity data, and candidate biomarkers exhibiting a difference greater than 1,500 RFU were selected for inclusion in subsequent validation studies. The overlap in candidate SLE biomarkers identified using these three statistical approaches is depicted in FIG. 3C.

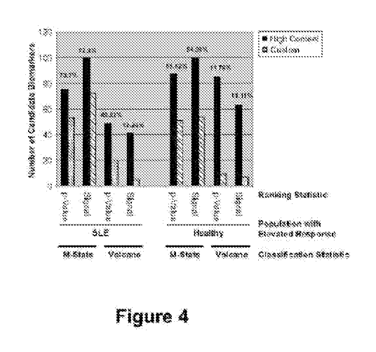

Array elements were spotted as adjacent duplicates in a 12-step, two-fold dilution series, and the resulting microarrays were probed with the original 30-sample panel, to evaluate the effectiveness of the different statistical approaches. Background-subtracted pixel intensity values were extracted from immune profiling experiments using these validation microarrays, and each array feature was subsequently classified as exhibiting elevated immune reactivity in either the SLE or the healthy population using M-statistics or volcano plot analysis as described above. Subsequent to this population assignment, array features were ranked by p-value or Signal Used difference. While a direct comparison of p-values was not possible because of the relatively small total number of features present on the validation microarrays, values calculated from the signal intensity data on these arrays were compared to the original high content array data to assess the reproducibility of immunoreactive signals. The number of proteins with a calculated p-value<0.01 or a Signal Used difference>1500 that were included on the validation arrays are indicated in FIG. 4 (solid bars). P-values and Signal Used differences calculated from the validation array data were used to generate a rank order, and proteins ranking in the top 100 on the validation arrays, sorted by either metric, are indicated with hatched bars. The percentage of proteins identified as significant in the original assays that are also in the top 100 on the custom arrays by either ranking statistic are indicated. The results of this analysis revealed a higher degree of validation between immunoreactivity observed on the original and validation microarrays for proteins eliciting an elevated immune response in the SLE population relative to the healthy population. The maximum validation rate in the SLE population was 72%, while the maximum validation rate observed in the healthy population was 58.6%. Additionally, proteins assigned to a population using M-statistics as the classification metric exhibited a higher degree of reproducibility relative to proteins assigned to a specific population using volcano analysis. The maximum validation rate observed for proteins classified by M-statistics was 72%, while the maximum validation rate observed for proteins classified by volcano analysis was only 40.8%. A global ranking scheme was developed for the list of candidate SLE biomarkers through the use of a scoring system in which proteins were assigned a point for each of the specified threshold criteria they met. The scoring metric factored in a number of statistical parameters including Z-factor, M-statistics p-value, Signal Used difference, and Signal Used ratio, with eighteen of the over 230 proteins generating the maximum score. Interestingly, 13 of these proteins were identified as SLE biomarkers by all three of the statistical approaches applied to the original data set, representing 50% of the proteins in the three-way zone of overlap (FIG. 3C). Further, all of these 18 candidate autoantigens were defined as hits using M-statistics, while 16/18 (89%) and 13/18 (72%) of the 18 autoantigens were identified using Signal Used difference and volcano analysis respectively. Taken together, these results suggest that M-statistics provides a robust analytical approach for identification of an initial set of putative autoantigens from high content protein microarray data sets.

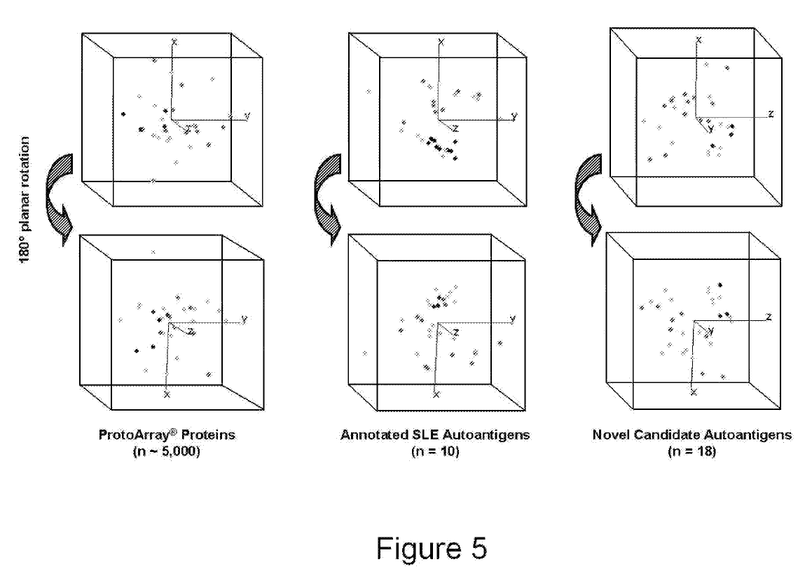

Principal Component Analysis (PCA) was used to qualitatively evaluate the separability of the two populations using either a panel of ten autoantigens that have been previously shown to be associated with SLE, or using the 18 candidate SLE biomarkers defined through the scoring analysis described above. The three-dimensional plots shown in FIG. 5 represent the first three principal components, and suggest that the novel SLE biomarkers defined through this study result in improved separation of the two populations relative to the separation achieved through Principal Component Analysis of the 10 literature-defined SLE antigens.

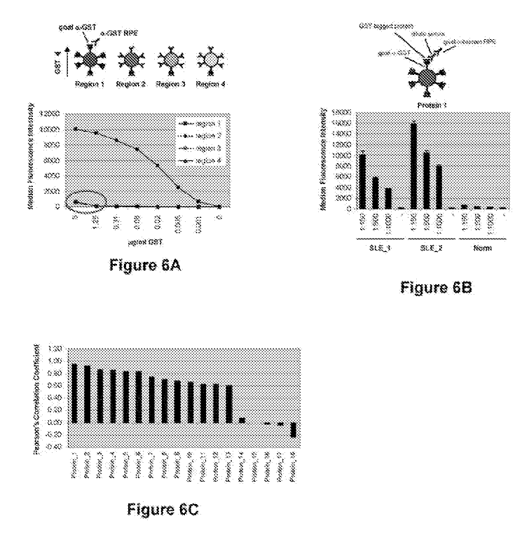

The results presented above demonstrated that the candidate biomarkers defined through the protein microarray assays exhibited reproducible reactivity when profiled on arrays comprised of proteins that were expressed and purified independently from those used in the original experiments. It was important, however, to validate the candidate biomarkers using an orthogonal technology. The Luminex® X-MAP technology was selected for these experiments as it is one of the few platforms that is suitable for carrying out multiplex assays in a clinical setting. A bead coupling strategy was utilized in which a goat anti-GST antibody was first conjugated to each bead region, enabling subsequent binding of the GST-tagged proteins. To evaluate the transfer of GST tagged proteins between beads post-coupling, purified GST was incubated with anti-GST-conjugated beads from one color region, and then mixed with anti-GST-conjugated beads from other color regions. As shown in FIG. 6A, the migration of GST from one anti-GST-conjugated bead region to another was not observed at GST protein concentrations below 5 μg/ml. Luminex® beads corresponding to 18 unique color regions were conjugated to goat anti-GST antibodies as described above, and then incubated separately with 1 μg/ml of each of the 18 candidate SLE biomarkers identified through the scoring analysis applied to the protein microarray data. All protein-bound bead regions were then combined, and incubated in triplicate with a dilution series of each of the sera utilized in the original study, including 20 samples from SLE patients and 10 samples from a healthy control population. These assays yielded reproducible, concentration-dependent signals (FIG. 6B). The relationship between the signals observed in the validation microarray assays and the signals observed in the Luminex assays were calculated using Pearson's correlation coefficient. In this experiment, signals for 13 of the 18 proteins (approximately 70%) yielded Pearson's Correlations>0.5 across all the study samples, similar to the rate of validation observed across the independent microarray experiments (FIG. 6C). Notably, nine of these 13 proteins correspond to proteins were derived from the 3-way zone of overlap resulting from the parallel statistical analysis originally carried out on the high content arrays. These results confirm and extend the findings of the autoimmune profiling carried out using protein microarrays by providing validation data using an unrelated technology platform.

### Example 6

Invitrogen's proprietary ProtoArray® Prospector software includes a series of algorithms specifically designed to analyze data resulting from immune response profiling studies, with the goal of identifying proteins that can be used to statistically differentiate two populations. A general overview of the process, as well as a detailed explanation of the specific algorithms is provided below.

The general approach used in Immune Response Profiling data analysis employs a three-step process:

Single array analysis: For each protein on each array, a series of values is calculated including background subtracted signals, Z-Score, Z-Factor, CI-P value, and replicate spot coefficient of variation (see below for details regarding the CI-P value)

Group characterization: Signals for each individual protein across all samples from a given population are aligned for downstream analysis

Identify differences between treated and untreated sample populations: Utilizing M-statistics, proteins are identified for which the differential signals between two populations result in a significant p-value

### CI-p-Value Calculation

The term CI-p-Value stands for Chebyshev's Inequality p-Value. The value is derived by testing the following hypothesis:

H₀: This spot comes from the Negative Control Distribution

Hₐ: This spot does not come from the Negative Control Distribution

In the effort to minimize assumptions about the negative control distribution, and hence the assumptions effects on the resulting p-values to test the given hypothesis, we utilize the Chebyshev's Inequality which states that if X is a random variable where μ=E(X) is the mean, σ²=Var(X) is the variance where if k>1 then,

$$P ( \frac{\vert X - μ \vert}{σ} \geq k ) \leq \frac{1}{{k}^{2}}$$

This is an absolute bound on the probability under the null hypothesis (this means that under the null hypothesis this is the most conservative p-value estimate). Again under the null hypothesis we assume that the non-control spot comes from the negative control distribution where we will estimate the sample mean and sample standard deviation are estimated from the signals from the negative controls. Using this Inequality we calculate the,

$$CI \text{-} p \text{-} Value = \{ \begin{matrix}1 & {Y}_{k} \leq \overline{X} + s \\ {( \frac{s}{( {Y}_{k} - \overline{X} )} )}^{2} & {Y}_{k} > \overline{X} + s\end{matrix}$$

where the mean and the standard deviation are from the observed signals in the Negative Control distribution. In this calculation Y represents the signal of the protein, s is the standard deviation of the negative controls, and k represents the kth protein, where Yk is the signal of the kth protein. Note that this is an upper bound on the true probability, since we are not making any assumptions of the distribution.

### Group Designation and Characterization

The purpose of this step is to provide the Prospector software with sample identities for a specified group of assays (e.g., those from “normal” individuals) and align background-subtracted signals calculated for each of these assays into a single file. This function takes as an input single microarray results calculated with Prospector, aligns values from the ‘Signal Used’ columns of single array analysis result files and writes the resulting spreadsheet into a single result file. The output is a tab-delimited text file with name starting with “Group Characterization Results”, which may be opened in Microsoft Excel.

### Group Comparison

The final set of algorithms compares two groups and identifies proteins which exhibit increased signal values in one group relative to another. M Statistics values are reported, which are described below. In addition, a p-value is calculated for each protein across a comparison that represents the probability that there is no signal increase in one group compared to another.

Analysis parameters include:

Quantile Normalization (default=on)—normalize signal values across assays being compared.

Signal must be larger than . . . RFUs (default is 500)—an additional parameter for M values calculation, which requires signal values to be over a specified background threshold;

Signal difference must be more then . . . RFUs (default is 200)—an additional parameter for M values calculation, which requires a specified gap between two signals to be considered significantly different.

Prospector reads specified group characterization files, completes calculations requested and writes resulting spreadsheet into a single result file. This tab-delimited text file, which may be opened in Microsoft Excel, contains a header detailing the analysis parameters applied. The result file contains a table with a list of probes with following columns of calculated values:

Group1 Count—The number of arrays in group 1 with signal larger than the cutoff

Group2 Count—The number of arrays in group 2 with signal larger than the cutoff

Group1 Prevalence—The estimated prevalence of the marker in group 1

Group2 Prevalence—The estimated prevalence of the marker in group 2

P-Value—The P-value for the most significant difference due to M statistic

Cutoff—The cutoff signal for determining a “hit”

Normalized Signal Values—if normalization was selected, columns with normalized data (one per array) are appended to the right.

### Calculations

### M-Statistics

This algorithm provides a count corresponding to the number of assays in one group for which a signal value for a specified protein is larger then the largest observed signal value for this protein in another group (smaller ellipse). The software subsequently calculates the number of arrays in a specified group with signals arising from this protein that are larger then the second largest signal in another group (larger ellipse), third largest etc., proceeding iteratively through the data set for all ProtoArray® proteins.

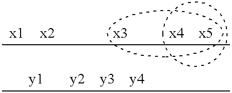

The M “I” order statistic for the group y of size n_{y} compared to group x of size nₓ is given by:

$${M}_{i , above , between}^{y} = {\sum }_{k = 1}^{{n}_{y}} {1}_{\{ {y}_{k} > {x}_{( i )} + between \}} {1}_{\{ {y}_{k} > above \}} \qquad ( 1 )$$

where x₍ᵢ₎ is the i^{th} largest value of the group x, and above and between are the calculation parameters.

The p-value is calculated as a probability of having an M value greater or equal then Mᵢ. Prospector selects the M statistic with the lowest p-value and reports this Mₘₐₓ value and order, as well as a corresponding p-value and prevalence estimate as described below.

Using a non-informative prior distribution for prevalence (i.e. assuming that the unknown prevalence of the marker is between 0 and 1) and acknowledging a binomial sampling scheme (i.e. that out of n arrays, the prevalence of the marker is given by p, one observes X arrays that are turned on), prevalence may be estimated as

$$E ( P ) = \frac{{M}_{max} + 1}{{n}_{y} + 2} . \qquad ( 2 )$$

### Quantile Normalization

Quantile normalization is a non-parametric procedure normalizing two or more one-channel datasets to a synthetic array. This method assumes that the distribution of signals is nearly the same in all samples. The largest signal for each array is replaced by a median value of the largest signals; the second largest signal is replaced by a median value of the second largest signals etc.

### Definitions of Statistical Terms

Hypothesis Testing: Two mutually exclusive hypotheses are given, one is typically called the null hypothesis and the other is typically called the alternative hypothesis. Data is then collected to test the viability of the null hypothesis, and this data is used to determine if the null hypothesis is rejected or not.

Rejection Rule: This is a statistical method in which the observed data either rejects the null hypothesis or fails to reject the null hypothesis. It is important to note that this Rule will never “accept the null or alternative hypothesis”; it is exclusively a rule to reject. There are four possible outcomes to this approach, based on the true nature of the null hypothesis, and what is decided by the Rejection Rule. The four outcomes can be shown as:

| | True Nature of H₀ | | |
|---|---|---|---|
| | | H₀ is True | H₀ is False |
| Decision by | Reject H₀ | Type I Error | Correct Decision |
| the Rejection | Fail to Reject | Correct | Type II Error |
| Rule | H₀ | Decision | |

Note that the true nature of H₀ is never really known. The actual formula for the Rejection Rule varies from hypothesis test to hypothesis test depending on the type of data, and the set of assumptions being made.

Type I Error: Typically, the probability of a Type I error is denoted as α. In general this is considered the most serious type of error to make.

Type II Error: Typically the probability of a Type II error is denoted as β. Though this is also an error, it is usually controlled by attempting to minimize the probability of Type I Error.

Precision: In a statistical terminology, precision is defined as the probability of not making a Type I Error. This can be considered as the probability of a true positive. Hence this is denoted as 1−α.

Power: In a statistical terminology, power is defined as the probability of not making a Type II Error. This can be considered the probability of a true negative. Hence this is denoted as 1−β.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

What is claimed is:

1. A method of detecting one or more target antibodies in a test sample of an individual suspected of having an autoimmune disease comprising:
a) contacting the test sample from the individual with one or more target antigens each comprising an autoantigen of Table 1 or a fragment thereof comprising an epitope recognized by a target antibody; and
b) detecting binding of one or more antibodies in the test sample to the one or more target antigens, thereby detecting the presence of the one or more target antibodies in the test sample.

2. The method of claim 1, wherein the one or more target antigens are immobilized on a solid support.

3. The method of claim 1, wherein the test sample is contacted with two or more target antigens of Table 1 or fragments thereof comprising an epitope.

4. The method of claim 1, wherein the test sample is contacted with twenty or more target antigens of Table 1 or fragments thereof comprising an epitope.

5. The method of claim 1, wherein the test sample is contacted with fifty or more target antigens of Table 1 or fragments thereof comprising an epitope.

6. The method of claim 1, wherein the test sample comprises cells, tissue, or a bodily fluid from the individual.

7. The method of claim 1, wherein the test sample comprises blood, serum, plasma, synovial fluid, cerebrospinal fluid, cell lysates or saliva from the individual.

8. The method of claim 1, further comprising detecting the amount of the one or more antibodies bound to the one or more target antigens in the test sample.

9. The method of claim 1, wherein binding of the one or more target antigens to one or more antibodies in the test sample is determined using an immunoassay.

10. The method of claim 1, wherein at least ten of the one or more target antigens are bound by the one or more antibodies from the test sample.

11. The method of claim 1, wherein at least twenty of the one or more target antigens are bound by the one or more antibodies from the test sample.

12. The method of claim 1, wherein at least fifty of the one or more target antigens are bound by the one or more antibodies from the test sample.

13. A method of diagnosing rheumatoid arthritis in an individual comprising:
a) contacting a test sample from the individual with one or more target antigens, each comprising an autoantigen of Table 2 or a fragment thereof comprising an epitope; and
b) detecting binding of the one or more target antigens to one or more antibodies in the test sample, wherein the presence of the one or more antibodies bound against the one or more target antigens is indicative of rheumatoid arthritis.

14. The method of claim 14, wherein the one or more target antigens comprises leukocyte receptor cluster member 12.

15. A method of diagnosing systemic lupus erythematosus in an individual comprising:
a) contacting a test sample from the individual with one or more target antigens, each comprising an autoantigen of Table 3 or fragments thereof comprising an epitope; and
b) detecting binding of the one or more target antigens to one or more antibodies in the test sample, wherein the presence of the one or more antibodies bound against the one or more target antigens is indicative of systemic lupus erythematosus.

16. The method of claim 15, wherein the test sample is contacted with one or more target antigens, each comprising an autoantigen of Table 4 or fragments thereof.

17. A method of diagnosing anti-neutrophil cytoplasmic antibody associated diseases in an individual comprising:
a) contacting a test sample from the individual with one or more target antigens, each comprising an autoantigen of Table 5 or fragments thereof comprising an epitope; and
b) detecting binding of the one or more target antigens to one or more antibodies in the test sample, wherein the presence of the one or more antibodies bound against the one or more target antigens is indicative of anti-neutrophil cytoplasmic antibody associated diseases.

18. The method of claim 17, wherein the test sample is contacted with one or more target antigens, each comprising an autoantigen of Table 6 or fragments thereof.

19. A kit for diagnosing an autoimmune disease comprising:
a) one or more target antigens each comprising an autoantigen of Table 1 or a fragment thereof comprising an epitope; and
b) means for detecting binding of one or more molecules in a test sample to the one or more target antigens.

20. The kit of claim 19, further comprising a control antibody against one or more of the target antigens.

21. The kit of claim 19, wherein the kit comprises two or more target antigens.

22. The kit of claim 19, wherein the kit comprises twenty or more target antigens.

23. The kit of claim 19, wherein the kit comprises fifty or more target antigens.

24. The kit of claim 19, wherein each of the one or more target antigens comprises an autoantigen of Table 2 or fragments thereof comprising an epitope.

25. The kit of claim 19, wherein each of the one or more target antigens comprises an autoantigen of Table 3 or fragments thereof comprising an epitope.

26. The kit of claim 19, wherein each of the one or more target antigens comprises an autoantigen of Table 5 or fragments thereof comprising an epitope.

27. The kit of claim 19, wherein the one or more target antigens are immobilized on one or more solid supports.

28. The kit of claim 19, wherein the one or more target antigens are part of a high density protein array.

29. The kit of claim 19, wherein the kit comprises less than 1,000 polypeptides.

30. The kit of claim 19, wherein the kit comprises less than 100 polypeptides.

31. A mixture comprising:
a) one or more target antigens each comprising an autoantigen of Table 1 or a fragment thereof comprising an epitope; and
b) a test sample from an individual suspected of having an autoimmune disease.

32. The mixture of claim 31, further comprising two or more target antigens of Table 1 or fragments thereof comprising an epitope.

33. The mixture of claim 31, further comprising twenty or more target antigens of Table 1 or fragments thereof comprising an epitope.

34. The mixture of claim 31, further comprising fifty or more target antigens of Table 1 or fragments thereof comprising an epitope.
